(19) United States
(12) Patent Application Publication
(10) Pub. No.: US 2005/0233406 A1
(43) Pub. Date: Oct. 20, 2005

(54) Methods for high resolution identification of solvent accessible amide hydrogens in polypeptides and for characterization of polypeptide structure

(76) Inventor: Virgil L. Woods JR., San Diego, CA (US)

Correspondence Address: FOLEY & LARDNER, P.O. BOX 80278, SAN DIEGO, CA, 92138-0278, US

(21) Appl. No.: 10/510,775

(22) PCT Filed: Apr. 10, 2003
(86) PCT No.: PCT/US03/11057

§ 371 Date: Jun. 9, 2005

Related U.S. Application Data
(60) Provisional application No. 60/371,366, filed on Apr. 10, 2002.
(60) Provisional application No. 60/400,614, filed on Aug. 1, 2002.

(51) Int. Cl. C12Q 1/37; G06F 19/00; G01N 33/48; G01N 33/50

(52) U.S. Cl. 435/23; 702/19

(57) ABSTRACT

The present invention provides methods of determining, at a resolution of about 1-5 amino acid residues, the position of a peptide amide hydrogen that has been labeled with an isotope of hydrogen other than ¹H by determining the quantity of isotope and/or rate of exchange of peptide amide hydrogen(s) with isotope. Invention methods comprise generating a population of sequence-overlapping endopeptidase fragments of the labeled protein under conditions of slowed hydrogen exchange and then deconvoluting fragmentation data. Invention methods allow for the localization of labeled peptide amide positions in an amino acid-specific manner, thereby providing information on residues involved in binding sites, surface conformation and accessibility of residues, and conformational changes at different times or conditions, for example.

### RELATED APPLICATIONS

This application claims the benefit of U.S. Provisional Application Ser. No. 60/400,614, filed Aug. 1, 2002, and U.S. Provisional Application Ser. No. 60/371,366, filed Apr. 10, 2002.

### FIELD OF THE INVENTION

The present invention relates to methods for characterizing polypeptide structure. In a particular aspect, the invention relates to improved methods for localizing labeled peptide amide hydrogens at high resolution using endoproteinase fragmentation. Invention methods are useful for a variety of applications, for example, determining solvent-accessibility of peptide amide groups, mapping binding interactions, and determining allosteric or conformational changes of a polypeptide, and the like.

### BACKGROUND OF THE INVENTION

Considerable experimental work and time are required to precisely characterize the structure of a polypeptide of interest. In general, the techniques that are the easiest to use and which give the quickest answers, result in an inexact and only approximate idea of the nature of the critical structural features. Techniques in this category include the study of proteolytically generated fragments of the protein which retain binding function; recombinant DNA techniques, in which proteins are constructed with altered amino acid sequence (for example, by site directed mutagenesis); epitope scanning peptide studies (construction of a large number of small peptides representing subregions of the intact protein followed by study of the ability of the peptides to inhibit binding of the ligand to receptor); covalent crosslinking of the protein to its binding partner in the area of the binding site, followed by fragmentation of the protein and identification of cross-linked fragments; and affinity labeling of regions of the receptor which are located near the ligand binding site of the receptor, followed by characterization of such “nearest neighbor” peptides.

In analyzing binding sites, such techniques work best for the determination of the structure of binding subregions which are simple in nature, as when a single short contiguous stretch of polypeptide within a protein is responsible for most of the binding activity. However, for many protein-binding partner systems of current interest, the structures responsible for binding on both receptor and ligand or antibody are created by the complex interaction of multiple non-contiguous peptide sequences. The complexities of these interactions may confound conventional analytical techniques, as binding function is often lost as soon as one of the 3-dimensional conformations of the several contributing polypeptide sequences is directly or indirectly perturbed.

The most definitive techniques for the characterization of the structure of polypeptides, and receptor binding sites in particular, have been NMR spectroscopy and X-ray crystallography. While these techniques can ideally provide a precise characterization of the relevant structural features, they have major limitations, including inordinate amounts of time required for study, inability to study large proteins, and, for X-ray analysis, the need for protein-binding partner crystals.

Hydrogen (Proton) Exchange

When a protein in its native folded state is incubated in buffers containing an isotope of hydrogen (for example, tritium or deuterium labeled water), isotope in the buffer reversibly exchanges with normal hydrogen present in the protein at acidic positions (for example, —OH, —SH, and —NH groups) with rates of exchange which are dependent on each exchangeable hydrogen's chemical environment, temperature, and most importantly, its accessibility to the isotope of hydrogen present in the buffer (see, e.g., Englander et al., Meth. Enzymol. 49: 24-39, 1978; Englander et al., Meth. Enzymol, 26:406-413, 1972). Accessibility is determined in turn by both the surface (solvent-exposed) dispositions of the hydrogen, and the degree to which it is hydrogen-bonded to other regions of the folded polypeptide. Simply stated, acidic hydrogen present on amino acid residues which are on the outside (buffer-exposed) surface of the protein and which are hydrogen-bonded to solvent water will often exchange more rapidly with heavy hydrogen in the buffer than will a similar acidic hydrogen which is buried and hydrogen-bonded within the folded polypeptide.

Hydrogen exchange reactions can be greatly accelerated by both add and base-mediated catalysis, and the rate of exchange observed at any particular pH is the sum of both add and base mediated mechanisms. For many acidic hydrogens, a pH of 2.2-2.7 results in an overall minimum rate of exchange (Englander et al., Anal. Biochem. 147:234-244, 1985; Englander et al., Biopolymers 7:379-393, 1969; Molday et al., Biochemistry 11:150, 1972; Kim et al., Biochemistry 21:1, 1982; Bai et al., Proteins: Struct. Funct. Genet. 17:75-86, 1993; and Connelly et al., Proteins: Struct. Funct. Genet. 17:87-92). While hydrogens in protein hydroxyl and amino groups exchange with tritium or deuterium in buffer at millisecond rates, the exchange rate of one particular acidic hydrogen, the peptide amide bond hydrogen, is considerably slower, having a half life of exchange (when freely accessible, and freely hydrogen-bonded to solvent water) of approximately 0.5 seconds at 0° C., pH 7, which is greatly slowed to a half life of exchange of 70 minutes at 0° C., pH 2.7. When a polypeptide is in a denatured, unstructured configuration (also termed a “random coil”) all of its amides can freely exchange with solvent hydrogen freely. However, the precise rate of exchange varies up to 200 fold from amide to amide under said unstructured conditions, the rate at each particular amide being determined by localized primary amino acid sequence-dependent effects that can be calculated from a knowledge of the peptide's primary sequence (Bai et al., supra).

When peptide amide hydrogens are buried within a folded polypeptide, or are hydrogen bonded to other parts of the polypeptide, exchange half lives with solvent hydrogens are often considerably lengthened, at times being measured in hours to days. Hydrogen exchange at peptide amides is a fully reversible reaction, and rates of on-exchange (solvent deuterium replacing protein-bound normal hydrogen) are identical to rates of off-exchange (hydrogen replacing protein-bound deuterium) if the state of a particular peptide amide within a protein, including its chemical environment and accessibility to solvent hydrogens, remains identical during on-exchange and off-exchange conditions.

Hydrogen exchange is commonly measured by performing studies with proteins and aqueous buffers that are differentially tagged with pairs of the three isotopic forms of hydrogen (¹H, normal hydrogen; ²H, deuterium; ³H, tritium). If the pair of normal hydrogen and tritium is employed, it is referred to as tritium exchange; if normal hydrogen and deuterium are employed, as deuterium exchange. Different physicochemical techniques are in general used to follow the distribution of the two isotopes in deuterium versus tritium exchange.

Tritium Exchange Techniques

Tritium exchange techniques (where the amount of the isotope is determined by radioactivity measurements) have been extensively used for the measurement of peptide amide exchange rates within an individual protein. The rates of exchange of other acidic protons (OH, NH, SH) are so rapid that they cannot be followed in these techniques and all subsequent discussion refers exclusively to peptide amide proton exchange. In these studies, purified proteins are on-exchanged by incubation in buffers containing tritiated water for varying periods of time, optionally transferred to buffers free of tritium, and the rate of off-exchange of tritium determined. By analysis of the rates of tritium on- and off-exchange, estimates of the numbers of peptide amide protons in the protein whose exchange rates fall within particular exchange rate ranges can be made. These studies do not allow a determination of the identity (location within the protein's primary amino acid sequence) of the exchanging amide hydrogens measured.

Extensions of these techniques have been used to detect the presence within proteins of peptide amides which experience allosterically-induced changes in their local chemical environment and to study pathways of protein folding (Englander et al., Meth. Enzymol. 26:406-413, 1972; Englander et al., J. Biol. Chem. 248:4852-4861, 1973; Englander, Biochemistry 26:1846-1850, 1987; Louie et al., J. Mol. Biol. 201:765-772, 1988). For these studies, tritium on-exchanged proteins are often allowed to off-exchange after they have experienced either an allosteric change in shape, or have undergone time-dependent folding upon themselves, and the number of peptide amides which experience a change in their exchange rate subsequent to the allosteric/folding modifications determined. Changes in exchange rate indicate that alterations of the chemical environment of particular peptide amides have occurred which are relevant to proton exchange (solvent accessibility, hydrogen bonding, etc.). Peptide amides which undergo an induced slowing in their exchange rate are referred to as “slowed amides” and if previously on-exchanged tritium is sufficiently slowed in its off-exchange from such amides there results a “functional tritium labeling” of these amides. From these measurements, inferences are made as to the structural nature of the shape changes, which occurred within the isolated protein. Again, determination of the identity of the particular peptide amides experiencing changes in their environment is not possible with these techniques.

Several investigators have described technical extensions (collectively referred to as medium resolution tritium exchange) which allow the locations of particular slowed, tritium labeled peptide amides within the primary sequence of small proteins to be localized to a particular proteolytic fragment, though not to a particular amino acid.

Rosa and Richards were the first to describe and utilize medium resolution tritium techniques in their studies of the folding of ribonuclease S protein fragments (Rosa et al., J. Mol. Biol. 133:399-416, 1979; Rosa et al., J. Mol. Biol. 145:835-851, 1981; and Rosa et al., J. Mol. Biol. 160:517-530, 1982). However, the techniques described by Rosa and Richards were of marginal utility, primarily due to their failure to optimize certain critical experimental steps. No studies employing related techniques were published until the work of Englander and co-workers in which extensive modifications and optimizations of the Rosa and Richards technique were first described.

Englander's investigations utilizing tritium exchange have focused exclusively on the study of allosteric changes which take place in tetrameric hemoglobin (a subunit and b subunit 16 kDa in size each) upon deoxygenation (Englander et al., Biophys. J. 10:577, 1979; Rogero et al., Meth. Enzymol. 131:508-517, 1986; Ray et al., Biochemistry 25:3000-3007, 1986; and Louie et al., J. Mol. Biol. 201:755-764, 1988). In the Englander procedure, native hemoglobin in the oxygenated state is on-exchanged in tritiated water. The hemoglobin is then deoxygenated (inducing allosteric change), transferred to tritium-free buffers by gel permeation column chromatography, and then allowed to out-exchange for 10-50 times the on-exchange time. On-exchanged tritium present on peptide amides which experience no change in exchange rate subsequent to the induced allosteric change in hemoglobin structure off-exchanges at rates identical to its on-exchange rates, and therefore is almost totally removed from the protein after the long off-exchange period. However, peptide amides which experience slowing of their exchange rate subsequent to the induced allosteric changes preferentially retain the tritium label during the period of off-exchange.

To localize (in terms of hemoglobin's primary sequence) the slowed amides bearing the residual tritium label, Englander then proteolytically fragments the off-exchanged hemoglobin with the protease pepsin, separates, isolates and identifies the various peptide fragments by reverse phase high pressure liquid chromatography (RP-HPLC), and determines which fragments bear the residual tritium label by scintillation counting. However, as the fragmentation of hemoglobin proceeds, each fragment's secondary and tertiary structure is lost and the unfolded peptide amides become freely accessible to H₂O in the buffer. At physiologic pH (>6), any amide-bound tritium label would leave the unfolded fragments within seconds. Englander therefore performs the fragmentation and HPLC peptide isolation procedures under conditions which minimize peptide amide proton exchange, including cold temperature (4° C.) and use of phosphate buffers at pH 2.7. This technique has been used successfully by Englander to coarsely identify and localize the peptidic regions of hemoglobin a and p chains which participate in deoxygenation-induced allosteric changes. The ability of the Englander technique to localize tritium labeled amides, while an important advance, remains low; at the best, Englander reports that his technique localizes amide tritium label to hemoglobin peptides 14 amino adds or greater in size, without the ability to further sublocalize the label. Moreover, in Englander's work, there is no appreciation that a suitably adapted exchange technique might be used to identify the peptide amides which reside in the contacting surface of a protein receptor and its binding partner: his disclosures are concerned with the mapping of allosteric changes in hemoglobin

Unfortunately, add proteases are very nonspecific in their sites of cleavage, leading to considerable HPLC separation difficulties. Englander tried to work around these problems, for the localization of hemoglobin peptides experiencing allosteric changes, by taking advantage of the fact that some peptide bonds are somewhat more sensitive to pepsin than others. Even then the fragments were “difficult to separate cleanly”. They were also, of course, longer (on average), and therefore the resolution was lower. Englander concludes, “At present the total analysis of the HX (hydrogen exchange) behavior of a given protein by these methods is an immense task. In a large sense, the best strategies for undertaking such a task remain to be formulated. Also, these efforts would benefit from further technical improvements, for example in HPLC separation capability and perhaps especially in the development of additional acid proteases with properties adapted to the needs of these experiments” (Englander et al., Anal. Biochem. 147:234-244,1985).

Over the succeeding years since this observation was made, no advances have been disclosed which address these critical limitations of the medium resolution hydrogen exchange technique. Most acid-reactive proteases are in general no more specific in their cleavage patterns than pepsin and efforts to improve the technology by employing other acid reactive proteases other than pepsin have not significantly improved the technique.

Allewell and co-workers have disclosed studies utilizing the Englander techniques to localize induced allosteric changes in the enzyme Escherichia coli aspartate transcarbamylase (Burz et al., Biophys. J. 49:70-72, 1986; Mallikarachchi et al., Biochemistry 28:5386-5391, 1989). Burz et al. is a brief disclosure in which the isolated R2 subunit of this enzyme is on-exchanged in tritiated buffer of specific activity 100 mCi/ml, allosteric change induced by the addition of ATP, and then the conformationally altered subunit off-exchanged. The enzyme R2 subunit was then proteolytically cleaved with pepsin and analyzed for the amount of label present in certain fragments. Analysis employed techniques that rigidly adhered to the recommendations of Englander, utilizing a single RP HPLC separation in a pH 2.8 buffer.

ATP binding to the enzyme was shown to alter the rate of exchange of hydrogens within several relatively large peptidic fragments of the R2 subunit. In a subsequent more complete disclosure (Mallikarachchi, supra), the Allewell group discloses studies of the allosteric changes induced in the R2 subunit by both ATP and CTP. They disclose on-exchange of the R2 subunit in tritiated water-containing buffer of specific activity 22-45 mCi/ml, addition of ATP or CTP followed by off exchange of the tritium in normal water-containing buffer. The analysis comprised digestion of the complex with pepsin, and separation of the peptide fragments by reverse phase HPLC in a pH 2.8 or pH 2.7 buffer, all of which rigidly adheres to the teachings of Englander. Peptides were identified by amino acid composition or by N-terminal analysis, and the radioactivity of each fragment was determined by scintillation counting. In both of these studies the localization of tritium label was limited to peptides which averaged 10-15 amino acids in size, without higher resolution being attempted.

Beasty et al. (Biochemistry 24:3547-3553, 1985) have disclosed studies employing tritium exchange techniques to study folding of the α subunit of E. coli tryptophan synthetase. The authors employed tritiated water of specific activity 20 mCi/ml, and fragmented the tritium labeled enzyme protein with trypsin at a pH 5.5, conditions under which the protein and the large fragments generated retained sufficient folded structure as to protect amide hydrogens from off exchange during proteolysis and HPLC analysis. Under these conditions, the authors were able to produce only 3 protein fragments, the smallest being 70 amino acids in size. The authors made no further attempt to sublocalize the label by further digestion and/or HPLC analysis. Indeed, under the experimental conditions they employed (they performed all steps at 12° C. instead of 4° C., and performed proteolysis at pH 5.5 instead of pH in the range of 2-3), it would have been impossible to further sublocalize the labeled amides by tritium exchange, as label would have been immediately lost (off-exchanged) by the unfolding of subsequently generated proteolytic fragments at pH 5.5 if they were less than 10-30 amino acids in size.

Fromageot et al., U.S. Pat. No. 3,828,102 discloses using hydrogen exchange to tritium label a protein and its binding partner, and Benson, U.S. Pat. Nos. 3,560,158 and 3,623,840 disclose using hydrogen exchange to tritiate compounds for analytical purposes.

However, none of the methods described in the art are capable of localizing the positions of the tritium labels of the labeled proteins at high resolution, the best resolution in the art generally being on the order of ≧14 amino acid residues.

Deuterium Exchange Techniques

Fesik et al. (Biochem. Biophys. Res. Commun. 147:892-898, 1987) disclose measuring by NMR the hydrogen (deuterium) exchange of a peptide before and after it is bound to a protein. From this data, the interactions of various hydrogens in the peptide with the binding site of the protein are analyzed.

Paterson et al. (Science 249:755-759, 1990) and Mayne et al. (Biochemistry 31:10678-10685, 1992) disclose NMR mapping of an antibody binding site on a protein (cytochrome-C) using deuterium exchange. This relatively small protein, with a solved NMR structure, is first complexed to anti-cytochrome-C monoclonal antibody, and the preformed complex then incubated in deuterated water-containing buffers and NMR spectra obtained at several time intervals. The NMR spectra of the antigen-antibody complex is examined for the peptide amides which experience slowed hydrogen exchange with solvent deuterium as compared to their rate of exchange in uncomplexed native cytochrome-C. Benjamin et al. (Biochemistry 31:9539-9545,1992) employ an identical NMR-deuterium technique to study the interaction of hen egg lysozyme (HEL) with HELspecific monoclonal antibodies. While both this NMR-deuterium technique, and medium resolution tritium exchange rely on the phenomenon of proton exchange at peptide amides, they utilize radically different methodologies to measure and localize the exchanging amides. Furthermore, study of proteins by the NMR technique is not possible unless the protein is small (generally less than 30 kDa), large amounts of the protein are available for the study, and computationally intensive resonance assignment work is completed.

Subsequently, others have disclosed techniques in which exchange-deuterated proteins are incubated with binding partner, off-exchanged, the complex fragmented with pepsin, and deuterium-bearing peptides identified by single stage fast atom bombardment (Fab) or electrospray mass spectroscopy (MS) (Thevenon-Emeric et al., Anal. Chem. 64-2456-2358, 1992; Winger et al., J. Am. Chem. Soc. 114:5897-5989, 1992; Zhang et al., Prot. Sci. 2:522-531, 1993; Katta et al., J. Am. Chem. Soc. 115:6317-6321, 1993; and Chi et al., Org. Mass Spectrometry 7:58-62, 1993; Engen and Smith, Anal. Chem. 73:256A-265A, 2001; Englander et al., Protein Sci. 6:1101-1109, 1997; Dharmasiri and Smith, Anal. Chem. 68:2340-2344, 1996; Smith et al., J. Mass Spectrometry 32:135-146, 1997; Deng and Smith, Biochemistry 37:6256-6262, 1998). In these studies, only the enzyme pepsin is employed to effect enzymatic fragmentation under slowed exchange conditions, and no attempt made to increase the number and quantity of useful fragments produced and studied beyond employing the methods disclosed by Englander and colleagues some decades prior. The resolution of the deuterium-exchange mass spectrometry work disclosed in these publications therefore remained at the 10-14 amino acid level, with the primary limitation of their art being the ability to generate only a small number of peptides with the endoproteinase pepsin, as they employed it. See FIG. 19 for an overview of this method of exchanged deuterium localization.

U.S. Pat. Nos. 5,658,739; 6,291,189; and 6,331,400 issued to Woods, Jr. (each of which is hereby incorporated by reference herein in its entirety), disclose improved methods of determining polypeptide structure and binding sites utilizing hydrogen-exchange-labeled peptide amides, importantly including a method of increasing the resolution of the technique to the 1-5 amino acid level. This increased ability to more precisely localize exchanged amide hydrogens was afforded by the novel use of acid-resistant carboxypeptidases to effect a subsequent progressive sub-fragmentation of the small number of relatively large-sized pepsin-generated peptides initially produced in the method (see FIG. 20 for an overview of the progressive proteolysis method). In these prior methods, finer localization of the labels is achieved by analysis of subfragments generated by controlled, stepwise, sub-degradation (“progressive degradation”) of each pepsin-generated, labeled peptide under slowed exchange conditions. According to these prior methods, the protein or a peptide fragment is said to be “progressively”, “stepwise” or “sequentially” degraded if a series of fragments are obtained which are similar to those that would be achieved an ideal exopeptidase. Carboxypeptidase-P, carboxypeptidase Y, and several other acid-reactive (i.e., enzymatically active under acid conditions) carboxypeptidases are specified for use in said progressive degradation of peptides under acidic conditions. To date, no aminopeptidases have been reported that are acid resistant; as a practicality, the only exopeptidases known or likely to be useful for this method are therefore carboxypeptidases.

Thus, carboxypeptidase digestion is of central importance and utility to these prior methods, and described therein as an essential step of the methods. Moreover, the prior art teaches that refinement of endopeptidase digestion methods under slowed exchange conditions, including that of pepsin, would be unlikely to prove successful (used alone) in significantly increasing useful fragmentation and therefore produce increased resolution in deuterium label localization. In particular, the prior art teaches that to produce even the low level of useful fragmentation observed, the enzyme fragmentation times employed with pepsin were already at the maximal duration allowable under quench conditions and that additional time taken for digestion with additional, even slower acid-resistant endoproteinases would produce unacceptable back-exchange deuterium losses. Thus, using the prior art methods, it would not be thought feasible to produce a dramatic increase in fragmentation within the available time for said digestions under exchange quench conditions, as the prior art regards digestions of more than 10 minutes as producing unacceptable losses of label through back-exchange with solvent.

While progressive proteolysis with the use of carboxypeptidases resulted in a considerable advance in the ability to more precisely localize exchanged deuterium at the time, it was at the cost of approximately doubling the work and time required for analysis. Carboxypeptidases operating under exchange-quench conditions can generally proceed through a maximum of 5-15 amino add residues within the allowable 10 minutes. Thus, it is therefore necessary to first digest the target proteins larger than this (the vast majority) with endoproteinases to produce fragments of this size prior to subjecting them to the action of carboxypeptidases. For practical purposes, this usually requires the processing and analysis of duplicate sets of samples: one set to establish endopeptidase fragmentation conditions, and another to establish hybrid endopeptidase-carboxypeptidase fragmentation conditions. Lesser but significant practical shortcomings of the use of carboxypeptidases are that only one has been identified to date that operates sufficiently well under quench conditions, (carboxypeptidase-P) and this one functions only at the basic end (above pH 2.7) of the acceptable pH range for slowed exchange, significantly away from the optimum slowed pH of 2.2 when LC-MS “friendly” buffers, such as the presently employed TFA and formic add buffers, are used. All of the acid-resistant endopeptidases in use for localization purposes (for example, pepsin, Newlase, Fungal protease XIII) work very well at pH 2.2. This means that the use of carboxypeptidases instead of these endoproteinases necessarily incurs a further loss of deuterium signal seen in further analysis, for example using mass spectrometry. Additionally, carboxypeptidase-P is presently more than fifty times more costly to obtain (on both an activity and weight basis) than the endopeptidases commonly employed for these studies. Thus, there remains a need in the art for improved (simpler, faster, more efficient, and cheaper) methods whereby the positions of labeled peptide amides can be determined within a resolution of 1-5 amino acid residues.

Thus, there remains a need in the art for improved simple, quick and efficient methods whereby the positions of labeled solvent-accessible peptide amide hydrogens can be localized at high resolution, particularly within 1-5 amino acid residues, within the primary amino acid sequence of a polypeptide or protein, as well as simple and efficient methods for studying or mapping polypeptide fine structure, such as binding sites and/or interaction surfaces of a polypeptide or protein.

### SUMMARY OF THE INVENTION

The present invention provides improved methods by which peptide amide hydrogen exchange can be used to characterize protein structure, dynamics, binding interactions and solvent accessibility. The methods of the present invention provide improvements and simplification of the protein degradation and fragmentation methods (performed under amide hydrogen exchange quench-conditions) previously described (U.S. Pat. Nos. 5,658,739; 6,331,400, and 6,291,189) for performing such characterizations, and, in particular, improved methods for calculating the amount of deuterium label at sequence-specific locations within a functionally labeled protein by analysis of deuterium-labeled protein fragmentation data. The elimination of unnecessary steps and improved deconvolution methods substantially increases throughput of the overall technique, without loss of the ability to highly localize deuterium label. The methods of the present invention are illustrated in FIG. 21.

The simplification omits the progressive degradation steps of the prior art methods (employing carboxypeptidases), which were previously thought critical to the success of high resolution localization of deuterium, and instead employing refined and enhanced methods by which endoproteinases can be made to rapidly produce highly varied and high yield, and highly sequence-overlapping protein fragmentation under optimal slowed exchange conditions. With this improved methodology, exchanged peptide amide hydrogens can be localized at high resolution (within 1-5 amino add residues) within the primary amino add sequence of a polypeptide or protein. The improvement affords simple and efficient methods for studying or mapping polypeptide fine structure, such as binding sites and/or interaction surfaces of a polypeptide or protein.

According to a first aspect of the present invention, there are provided methods of determining, at a resolution of about 1-5 amino acid residues, the position of peptide amide hydrogen(s). Such methods comprise labeling a protein of interest with an isotope of hydrogen other than ¹H, such as deuterium, and determining the quantity of isotope and/or rate of exchange of hydrogen at a peptide amide hydrogen(s) with the isotope by generating a population of sequence-overlapping endopeptidase fragments of said labeled protein under conditions of slowed hydrogen exchange and then deconvoluting fragmentation data acquired from the resultant endopeptidase fragments.

In preferred embodiments, invention methods measure the mass of peptide fragments, for example, utilizing mass spectrometry, to determine the presence or absence and/or quantity of an isotope of hydrogen on an endopeptidase fragment. Fragmentation data is deconvoluted by comparing the quantity and rate of exchange of isotope(s) on a plurality of sequence-overlapping endopeptidase fragments with the quantity and rate of exchange of isotope(s) on at least one other endopeptidase fragment, wherein said quantities are corrected for back-exchange in an amino add sequence-specific manner.

According to another aspect of the present invention, there are provided methods of characterizing the binding site of a binding protein. These methods also employ peptide amide hydrogen exchange and fragmentation analysis with endopeptidases.

According to yet another aspect of the present invention, there are provided methods of determining peptide amides that are near residues important in the interaction between a binding protein and a high affinity binding partner therefor. These methods could in turn be utilized in methods of screening test compounds to determine whether any compounds mimic the interaction of a binding protein and a high affinity-binding partner therefor.

According to additional aspects of the present invention, there are provided methods of determining the surface conformation of a polypeptide, and of determining a conformation change in a polypeptide. These methods also employ peptide amide hydrogen exchange and fragmentation analysis with endopeptidases.

In order to determine rates of exchange and to localize the position of incorporated isotope in an amino acid specific manner, a densely amino acid sequence-overlapping population of endopeptidase fragments of the labeled protein is generated. The precise location, within the protein, of each peptide amide that is functionally labeled by virtue of its solvent accessibility and/or its interaction with its binding partner is determined by analysis of the label content of the many overlapping endopeptidase fragments. Inferentially, in this manner, the precise amino acids that make up the surface of the receptor and/or the surface of the receptor's binding site are then also known Studies may be performed to quantify the exchange rates of each of the labeled amides identified above both before and after complex formation with binding partner. This allows calculation of the magnitude of exchange slowing experienced by each of these amides consequent to complex formation, and thereby exchange protection factors.

### BRIEF DESCRIPTION OF THE FIGURES

FIG. 1 collectively shows the primary and secondary structure of human D-AKAP2. FIG. 1A illustrates the predicted domain organization of D-AKAP2, containing two putative regulators of G protein signaling (RGS) domains, a PKA kinase binding (AKB) domain, and a PDZ binding motif at the C terminus. A consensus PKA phosphorylation site is indicated between RGS B and the AKB domains. The bold line indicates the region of D-AKAP2 used in methods of the present invention. FIG. 1B shows a Clustal W sequence alignment of RGS4, RGS A and RGS B of D-AKAP2. An asterisk (*) indicates the identical residues and the residues comprising the hydrophobic core are boxed. The secondary structure corresponding to RGS4 is indicated above the alignment.

FIG. 2 depicts the binding of D-AKAP2 to the regulatory subunit (RIIα) of PKA as determined using surface plasmon resonance. A total of 450 response units of RIIα were immobilized to a cAMP sensor chip. Serial dilutions (250-2 nM) of D-AKAP2 were injected over the surface. Both the one rate (kₒₙ) and the off rate (k_{off}) were fit globally using the concentration series. A 1:1 binding model fits the data well and is illustrated as a bold line.

FIG. 3 is a pepsin digestion map of D-AKAP2. A light line indicates a peptide that was identified and a dark line indicates a peptide that used for DXMS analysis.

FIG. 4 shows the deuteration levels of D-AKAP2 after on-exchange at 0° C. The deuteration level is indicated below the sequence as a colored bar (see the inset). The on-exchange time is listed to the left of the sequence. The residues corresponding to the split RGS A, RGS B, AKB and PDZ binding motif are indicated. Black arrows indicate limited proteolysis cleavage sites after one hour. Open arrows indicate limited proteolysis cleavage sites after 24 hours. The asterisk (*) above S267 indicates an in vitro phosphorylation site.

FIG. 5 collectively shows the modeling and deuteration levels of D-AKAP2 domains. FIG. 5A is a backbone structural alignment of the modeled RGS domain of D-AKAP2 and RGS4. FIG. 5B is a modeled RGS B domain of D-AKAP2 with deuteration levels after 3000 seconds on-exchange at 0° C. mapped onto the structure. FIG. 5C shows the deuteration levels of RGS B domain after 3000 seconds on-exchange at 0° C. Boxed characters indicate the conserved hydrophobic residues as in FIG. 1B.

FIG. 6 depicts sub-localization of the deuteuration levels of the AKB domain after on-exchange at 0° C. By subtracting two overlapping peptides, the protected region of the AKB domain was further extended to residues 349-360. The region corresponding to the AKB domain is indicated with a box. Asterisks (*) indicate residues that, when mutated, disrupt or abolish binding to the regulatory subunit of PKA.

FIG. 7 is a schematic of an exemplary automated DXMS apparatus useful in the methods of the present invention.

FIG. 8 illustrates peptide probes employed in the present study mapped to the primary sequence of Csk. The designated secondary structure, domains, and loop regions are based on the three-dimensional structure of Csk.

FIG. 9 shows the results of ESI-MS of pepsin-treated Csk before (upper panel) and after (lower panel) incubation with D₂O. The peak clusters corresponding to the peptide fragment, I255-L262, are shown expanded in the insets in the upper and lower panels.

FIG. 10 illustrates the time-dependent deuterium incorporation in several peptide probes in the absence and presence of nucleotides, comparing deuterium incorporation into apo-, AMPPNP-bound, and ADP-bound forms of Csk. The probes listed in the individual plots correspond to the following structural elements based on the X-ray coordinates of Csk: E93-L103 (αA in SH2), S139-V144, V184-N191 (SH2-kinase linker), L198-M210 (glycine-rich loop), L234-M240 (αC), V249-V254, F310-T321 (catalytic loop), V322-F333, G334-L358 (activation loop), and G383-G402 (αG).

FIG. 11 collectively shows the effects of nucleotide binding on Csk. FIG. 11A shows all probes used for H-D exchange studies mapped to the X-ray structure for Csk. Several key structural features are labeled. FIG. 11B shows the probes protected by AMPPNP and ADP. Probes in black show greater protection in the presence of ADP than AMPPNP. Probes in dark gray show greater protection in the presence of AMPPNP compared to ADP. Probes in medium gray are not protected by either nucleotide. Probes shown in light gray are equally protected from deuterium incorporation by AMPPNP and ADP.

FIG. 12 illustrates the fragmentation map of RIIβ.

FIG. 13 graphically illustrates the maximum change in % deuteration between cAMP-bound and C-subunit bound conformations of RIIβ. The domain boundaries are shown as dashed lines.

FIG. 14 collectively shows amide exchange of the dimerization/docking domain. FIG. 14A shows percent deuteration for residues 2-19. The top bar for each ligand state represents t=10 s and the bottom bar represents t=3000 s. Secondary structure assignments are labeled above the sequence. FIG. 14B plots the number of deuterons incorporated as a function of time for residues 15-19 in cAMP-free (●), cAMP-bound (▪), and C-subunit bound (▴) conformations of RIIβ.

FIG. 15 collectively shows amide exchange of the linker region. FIG. 15A shows percent deuteration for residues 28-130. The top bar for each ligand state represents t-10 s and the bottom bar represents t-3000 s. Secondary structure assignments are labeled above the sequence. FIG. 15 plots the number of deuterons incorporated as a function of time for residues 102-115 in cAMP-free (●), cAMP-bound (▪), and C-subunit bound (▴) conformations of RIIβ.

FIG. 16 collectively shows amide exchange of PBCs. FIG. 16A is a ribbon diagram of cAMP-binding domains highlighting residues 222-224 (cA:αP), 228-233 (cA:PBC), 341-353 (cB:β6/αP), and 354-363 (cB:PBC). FIG. 16B shows percent deuteration for residues of cAMP-binding pockets. The top bar for each ligand state represents t-10 s and the bottom bar represents t=3000 s. Secondary structure assignments are labeled above the sequence. FIG. 16C plots the number of deuterons incorporated as a function of time for sample residues 228-233 in cAMP-free (●), cAMP-bound (▪), and C-subunit bound (▴) conformations of RIIβ. This plot is representative of all 4 peptides.

FIG. 17 collectively shows amide exchange of cAMP-binding domain peptides showing increased deuteration upon C-subunit binding. FIG. 17A is a ribbon diagram of the cB domain highlighting residues 303-312 (cB:β3) 321-325 (cB:β4), 377-379 (cB:αB) 390-396 (cB:αC), and 399-401 (cB:αC). FIG. 17B shows percent deuteration for each ligand state at t=10 s (top bar) and t3000 s (bottom bar). Secondary structure assignments are labeled above the sequence. FIG. 17C plots the number of deuterons incorporated as a function of time in cAMP-free (●), cAMP-bound (▪), and C-subunit bound (▴) conformations of RIIβ for residues 390-396 and 399401. The plot for residues 390-396 is representative of the remaining 3 peptides.

FIG. 18 collectively shows amide exchange for cAMP-binding domain peptides showing decreased deuteration upon C-subunit binding. FIG. 18A is a ribbon diagram of cAMP-binding domains highlighting residues 150-152 (αXₙ′), 253-268 (cA:αC,αC′), 271-277 (cA:αC″), 278-281 (cB:αA), and 381-387 (cB:αB/αC). FIG. 18B shows percent deuteration for each ligand state at t=10 s (top bar) and t=3000 s (bottom bar). Secondary structure assignments are labeled above the sequence. FIG. 18C plots the number of deuterons incorporated as a function of time for cAMP-free (●), cAMP-bound (▪), and C-subunit bound (▴) conformations of RIIβ.

FIG. 19 outlines an exemplary prior art deuterium label localization method. Given the limited ability of pepsin to produce varied fragments under these conditions, the goal of the method was to produce one peptide from each region of the target protein's primary sequence.

FIG. 20 depicts the prior art progressive degradation method for deuterium localization in which a target protein is first subjected to endopeptidase-dependent fragmentation as in FIG. 19, followed by carboxypeptidase-dependent sub-fragmentation.

FIG. 21 depicts a simplified schematic of exemplary methods of the present invention, in which a densely overlapping set of target protein fragments is generated in a single step by enhanced use of endoproteinases. In this schematic, the functionally labeled protein was tagged with two sequence-discontinuous deuterons, and every deuterated peptide produced by subsequent endopeptidase fragmentation was found to bear one deuteron.

FIG. 22 illustrates the effect of varying concentrations of guanidine hydrochloride (0-4 M GuHCl) on the fragmentation of protein phospholipase A2, under quench conditions (pH 2.2, 0° C.), with solid-phase pepsin. When no GuHCl was used, less than 15 peptides in sufficient yield were identified. When 0.05 M GuHCl was used, more than 70 high quality peptides were obtained, with further improvement when 0.5 M denaturant was employed. While 4.0 M denaturant resulted in the identification of additional peptides, some were also lost when compared to the 0.5M digest.

FIG. 23 presents results obtained when horse cytochrome c was quenched with or without 0.5 M GuHCl, and then fragmented with either pepsin (P1), Newlase (P2) or Fungal Protease XIII (P3) coupled to perfusive supports (20-30 mg/ml). Considerable variation in digestion pattern and yield is seen with the varying conditions. The arrows at the top of the figure indicates the positions of the C- and N-termini of the aggregate peptides produced, highlighting the extreme degree of overlap of the set of peptide fragments produced.

FIG. 24 presents the fragmentation map obtained for a human von Willebrand Factor construct (denatured in 0.5 M GuHCl) employing a 40 second digestion on a pepsin column. In this study it was necessary to simultaneously reduce an internal disulfide bond by mixing TCEP (1.0 M final concentration) with the denaturant.

FIG. 25 presents the results of a deconvolution of fragmentation data obtained from chicken brain spectrin analyzed according to methods of the present invention. The deuterium content of the 113 useful peptides resulting from such fragmentation was determined from the raw MS data. Plots of deuterium buildup versus time were constructed for each peptide, and the number of amides exchanging in arbitrary “fast, medium and slow” classes (light, medium, and dark grey colors respectively in the figure) determined for each peptide. An initial map of rates versus amino acid sequence was then constructed from this information, employing a strategy in which “pieces” (fragments) with uniform rate class (color), were first placed in register, and subsequent placement of more complexly colored pieces (two color then three color), and performed in a manner that required that the several “colors” in these peptides be reconciled vertically to conform with color placement of the preceding pieces. The average color (rate class) at each amide position was then determined and used to construct the initial map. Unmeasurable amide hydrogens (approximately 10% of the total amides in the 113 fragments, unmeasured either because of errors incurred because of the approximate (average) back-exchange calculation method employed, or because the very slowest exchanging amides were not measured in this experiment) were then fit to the provisional map in a manner that minimized deviation from said map, and a final map constructed by averaging this final placement of “pieces”.

### DETAILED DESCRIPTION OF THE INVENTION

In a first aspect, the present invention provides methods of determining, at a resolution of about 1-5 amino add residues, the position of a peptide amide hydrogen that has been labeled with an isotope of hydrogen other than ¹H within a protein of interest, said method comprising: determining the quantity of isotope and/or rate of exchange of peptide amide hydrogen(s) labeled with said isotope by generating a population of sequence-overlapping endopeptidase fragments of said labeled protein under conditions of slowed hydrogen exchange and then deconvoluting fragmentation data acquired from said endopeptidase fragments.

A protein or polypeptide of interest is first labeled with an isotope of hydrogen other thin ¹H, for example deuterium (²H) or tritium (³H). This labeling is accomplished under essentially physiologic conditions by incubating the protein of interest in solutions substantially containing water composed of the isotope. The phenomenon of hydrogen exchange is used to substitute an isotope of hydrogen for at least one of the amide hydrogens on the amino adds of the protein of interest.

The term “protein” or “polypeptide” is used herein in a broad sense, which includes, for example, polypeptides and oligopeptides, and derivatives thereof, such as glycoproteins, lipoproteins, and phosphoproteins, and metalloproteins. The essential requirement is that the protein contains one or more peptide (—NHCO—) bonds, as the amide hydrogen of the peptide bond (as well as in the side chains of certain amino acids) has certain properties which lends itself to analysis by proton exchange. The protein may be identical to a naturally occurring protein, or it may be a binding fragment or mutant of such a protein. The fragment or mutant may have the same or different binding characteristic relative to the parental protein.

Solvent accessible peptide amide hydrogens of a polypeptide or protein of interest are on-exchanged by contacting the polypeptide or protein with an isotope of hydrogen under conditions wherein the native solvent-accessible peptide amide hydrogens are replaced with the isotope (for example, deuterium or tritium), such as, for example, physiological conditions wherein the polypeptide or protein is folded into its native conformation. Peptide amide protons that are relatively inaccessible to solvent, such as those that are buried within the interior of the polypeptide or protein structure or those that participate in intramolecular hydrogen-bonding interactions, do not readily exchange with the isotopic hydrogens in the solvent. Thus, those peptide amide hydrogens that are more solvent-accessible are selectively labeled.

The numerous small peptide fragments that are produced and analyzed by the methods of the present invention are likely to all be in random coil configuration: they are small, with little opportunity for structure-forming interactions, and are continuously contacted with several structure-breaking denaturants. According to certain invention methods, deuterated proteins are shifted to slowed exchange conditions (that include a very acidic pH), admixed with denaturing guanidinium salts, optionally disulfide-reduced, subject to proteolysis to generate a population of small fragments, and then admixed with acetonitrile, again under very acid conditions. As these fragments are in random coil configuration, the rates of exchange of each amide, in each peptide, under the slowed exchange (“quench”) conditions as employed herein can be calculated from a knowledge of the amino acid sequence of each fragment (Bai et al., supra) as well as determined experimentally by fragmentation-LC-MS analysis of initially equilibrium-deuterated protein or peptides. As demonstrated herein, such calculations and measurements are employed to provide precise corrections for deuterium losses from peptides that occur in the course of the analysis, and to provide an adjunctive method for further localizing deuterium on peptide amides, when the fragmentation data alone is insufficient to achieve the desired resolution

The protein of interest is first labeled under conditions wherein native hydrogens are replaced by the isotope of hydrogen (this is the “on-exchange” step). The reaction conditions are then altered to slowed hydrogen exchange conditions, or exchange “quench” conditions for further analysis of exchange rates. The phrase “slowed hydrogen exchange conditions” as used herein, refers to conditions where the rate of exchange of normal hydrogen for an isotope of hydrogen at amide hydrogens freely exposed to solvent is reduced substantially, i.e., enough to allow sufficient time to determine, by the methods described herein, exchange rates and the location of amide hydrogen positions which had been labeled with heavy hydrogen. The hydrogen exchange rate is a function of temperature, pH and solvent. The rate is decreased three fold for each 10° C. drop in temperature. In water, the minimum H-exchange rate is at a pH of 2-3. The use of a temperatures in the range of about 0-10° C., and a pH in the range of about 2-3 is preferred. Most presently preferred are conditions of about 0° C. and pH 2.2. As conditions diverge from the optimum pH, the H exchange rate increases, typically by 10-fold per pH unit increase or decrease away from the minimum. Use of high concentrations of a polar, organic cosolvent shifts the pH min to higher pH, potentially as high as pH 6 and perhaps, with certain solvents, even higher.

At pH 2.2 and 0° C., the typical half-life of a deuterium label at an amide position freely exposed to solvent water is about 70 minutes. Preferably, the slowed conditions of the present inventions result in a half-life of at least 10 minutes, more preferably at least 60 minutes.

To achieve labeling of the protein of interest, the protein is incubated in buffer supplemented with deuterated water (preferably ²H₂O), preferably of high concentration, preferably greater than 25% mole fraction deuterated water. This results in the time dependent reversible incorporation of deuterium label into every peptide amide on the surface of the protein through the mechanism of hydrogen exchange. These amides are referred to herein as “solvent accessible”. Suitable buffers include phosphate buffered saline (PBS), 0.15 mM NaCl, 10 mM PO₄ (pH 7.4). The use of small incubation volumes (about 0.1-10 μl) containing high concentrations of protein (about 2-10 mg/ml) is preferred. This can be done, for example, by adding protein and buffer together in a tube, or by injecting an aliquot of protein solution into a flowing stream of isotope-containing buffer in a manner that results in the rapid mixing of the converging streams.

It is not necessary that the hydrogen exchange analysis rely on only a single choice of “on-exchange” time. Rather, the skilled worker may carry out the experiment using a range of on-exchange times, preferably spanning several orders of magnitude (seconds to days) to allow selection of on-exchange times which allow efficient labeling of the various peptide amides present in the protein, and at the same time minimize background labeling of other amide positions after off-exchange is completed.

In general comparisons of the exchange behavior of alternative forms of a protein can be performed by either: (i) on-exchanging, in parallel, each of the forms of the protein, quenching exchange, performing localization studies on each form of the protein, and then comparing the deuteration patterns seen across the set of protein forms; and (ii) on-exchanging one form of the protein, transforming the protein to its alternative form (for example, inducing a conformation change, binding a ligand, etc.) and then off-exchanging the protein, said off-exchange terminated by quenching exchange. In both methods of analysis, the ration of the exchange rates observed at any amide position is termed its exchange “protection factor”, and this ratio is related to the change in free energy (“delta G”) in the atomic environs of said amide by the relationship Delta G=−T In (protection factor).

For off-exchange, the labeled protein is transferred to physiologic buffers identical to those employed during on-exchange, but which are substantially free of isotope. The incorporated isotopic label on the protein then exchanges off the protein at rates identical to its on-exchange rate everywhere except at amides that have been slowed in their exchange rate, for example, by virtue of the interaction of protein with a binding partner, or by conformational change.

In general, off-exchange is allowed to proceed for 2 to 20 times longer, more preferably about 10 times longer, than the on-exchange period, as this allows off-exchange from the protein of greater than 99% of the on-exchanged isotope label.

In preferred embodiments, the off-exchange procedure may be accomplished by use of perfusive HPLC supports that allow rapid separation of peptide/protein from solvent (Poros™ columns, PerSeptive Biosystems, Boston, Mass.), or by simple dilution into undeuterated solvent.

According to a particular aspect of the present invention, there are provided methods of characterizing the binding site of a binding protein, said method comprising: labeling said protein with an isotope of hydrogen other than ¹H in the presence and absence of a binding partner for said protein, and comparing the pattern of labeling obtained on said protein in the presence and absence of said binding partner, wherein the location of label is determined by generating a population of sequence-overlapping endopeptidase fragments of said labeled protein under conditions of slowed hydrogen exchange and then deconvoluting fragmentation data acquired from said endopeptidase-generated fragments.

Many biological processes are mediated by noncovalent binding interactions between a protein and another molecule, its binding partner. Such binding partners include, for example, other polypeptides, nucleic acids, or small molecules such as ligands, substrates, inhibitors, activators, co-factors and drugs. The identification of the structural features of the two binding molecules that immediately contribute to those interactions would be useful in designing drugs that alter these processes.

The molecules that preferentially bind each other may be referred to as members of a “specific binding pair”. Such binding pairs include an antibody and its antigen, a lectin and a carbohydrate that it binds, an enzyme and its substrate, and a hormone and its cellular receptor. In some texts, the terms “receptor” and “ligand” are used to identify a pair of binding molecules. Usually, the term “receptor” is assigned to a member of a specific binding pair that is of a class of molecules known for its binding activity, e.g., antibodies. The term “receptor” is also preferentially conferred on the member of the pair that is larger in size, e.g., on avidin in the case of the avidin-biotin pair. However, the identification of receptor and ligand is ultimately arbitrary, and the term “ligand” may be used to refer to a molecule which others would call a “receptor”. The term “anti-ligand” is sometimes used in place of “receptor”.

A “binding site” is a point of contact between a binding surface of the binding protein and a complementary surface of the binding partner. For example, in the case of antibody-antigen interactions, it is conventional to refer to the antigen binding site of the antibody as the “paratope” and the target site on the antigen as the “epitope”. A specific binding pair may have more than one binding site, and the term “pair” is used loosely, as the binding protein may bind two or more binding partners (for example, as in the case of a divalent antibody). Moreover, other molecules, e.g., allosteric effectors, may alter the conformation of a member of the “pair” and thereby modulate the binding. The term “pair” is intended to encompass these more complex interactions.

To characterize a binding site of the labeled protein, binding partner is subsequently added to the on-exchanged labeled protein and allowed to bind. Once the binding partner has bound to the protein, hydrogens on the amino adds which make up the surface of the receptor's binding site are no longer capable of efficiently interacting with the surrounding aqueous buffer, and further hydrogen exchange at peptide amides within the binding site is markedly inhibited. These positions will thus be identifiable by a slowed amide hydrogen exchange rate.

In a preferred method, the labeled protein is contacted with a support matrix to which a binding partner has previously been physically attached. The on-exchange mixture is contacted with the support matrix-binding partner for a time sufficient for complex formation to occur, typically seconds to a minute, and then the support matrix bearing attached bound binding protein-binding partner complex washed with solvents devoid of isotope label, affecting initiation of binding protein off-exchange. Such support-bound binding partner can additionally be employed to form a complex of binding partner and binding protein, which can then be contacted with deuterated water to initiate on-exchange while bound to the support. Such support-bound binding partner can also be used to quickly collect and concentrate binding protein that has been on-exchanged by simple dilution, as would be the control for the foregoing on-column deuteration of the complex.

Off-exchange can be terminated simultaneously with release of binding protein from the support matrix-binding partner by contacting the support matrix with solvents having properties sufficient to both disrupt the binding protein-binding partner bond, and also optimally slow amide hydrogen exchange, preferably by altering the binding protein pH to that of 2.2, and performing said elution at 0° C. This detached binding protein is eluted from the support matrix and then further processed with steps that may include denaturation, disulfide reduction and pepsin proteolysis.

In a preferred embodiment the support matrix with covalently bound binding partner is contained in an HPLC column that has an established stream of aqueous solvent flowing through it at 0° C. with pH and other solvent conditions conducive to binding protein-binding partner complex formation. At the end of the on-exchange interval, the aliquot of isotope-contacted binding protein is injected into this stream and rapidly entrained into the column where it admixes with the contained support matrix. As the stream continues to flow, any isotope in the solvent not attached to the binding protein will promptly exit the column (in the void volume) and, as off-exchange proceeds any heavy hydrogen that exchanges off the binding partner will be similarly removed from the support matrix and column by the flowing stream of aqueous solvent.

At the end of off-exchange, an aliquot of “elution solvent” is entrained into the flowing stream, having a buffering capacity and pH sufficient to shift the affinity matrix to a pH conducive to slowed exchange (approximately 2.2). Other additives including denaturants (guanidine thiocyanate at a concentration of about 0.5-3.0 M, preferably 2.0 M; guanidine hydrochloride at a concentration of about 1.0-3.0 M, preferably 2.0 M) or reductants (e.g. a phosphine such as TCEP at a concentration of about 0.1-2.0 M, preferably 1.0 M) may optionally be added as needed to effect release of the binding protein from the support matrix. With continued solvent flow, the binding protein leaves the support column and matrix in pure form, without admixed binding partner. It then optionally contacts further columns containing matrices appropriate for reduction and/or pepsin proteolysis as desired.

Determination of amide exchange rates in proteins requires performing studies across a broad range of on and off-exchange intervals. For brief on- and off-exchange intervals (1-2 minutes or less), the time necessary for binding protein to be applied to the matrix-containing column and both bind to binding partner and start off-exchange may be excessively long with the above approach. While the above approach will work well with on and off-exchange intervals as short as 1-2 minutes, limits to the ability of support matrices to promote the rapid molecular interaction of binding protein with binding partner will make study of exchange intervals shorter than this problematic with the above approach. While homogenous liquid phase reactions between a receptor and ligand may be quite fast (less than 1/10^{th} of a second), if one of the pair has been previously attached to a surface, then limitations to “transport processes” can substantially slow the binding interaction (to seconds).

To overcome this difficulty, the following modified approach is utilized for study of brief exchange intervals. Binding protein is contacted with isotope-containing solvent as above, but at the end of the desired on-exchange interval, the solution is contacted with a small volume of liquid phase binding partner. As both binding components are in homogenous liquid phase, binding the complex formation occurs at intervals well less than one second. An excess of aqueous solvent devoid of heavy hydrogen is then optionally added to the binding protein-binding partner complex mixture to effect a substantial dilution (1/10 to 1/1000, preferably 1/100) of the isotope in the mixture, thereby initiating off-exchange. This mixture is then rapidly applied to a support matrix column (preferably by the flowing stream method) that is capable of binding and attaching the binding partner by any of a variety of methods that are operative at physiologic pH, including the avidin-biotin interaction (in this case the binding partner having been previously biotinylated and the matrix support bearing previously attached avidin) or by way of other well characterized binding pair interactions.

Continued flow of solvent without isotope over the binding protein-binding partner-bound support matrix initiates off-exchange. At the end of off-exchange, binding protein is then eluted and removed from the column with an appropriate buffer capable of dissociating the binding protein-binding partner complex; the binding partner-solid support interaction; or both. Preferably one employs procedures that are capable of selectively disrupting the binding protein-binding partner complex without disrupting the support matrix-binding partner interaction (for example, the avidin-biotin interaction) as this will result in the preferred specific elution and recovery from the column of pure off-exchanged binding protein, unadulterated with confounding binding partner.

A preferred embodiment employs binding protein that is first contacted with isotope-containing solvent, and, at the end of the desired on-exchange interval, this solution is contacted with a solution of a previously biotinylated binding partner, with such prior biotinylation being accomplished by any of a number of well known procedures. Complex formation between biotinylated binding partner and binding protein is allowed to occur, generally being complete in less than a second, and then this mixture optionally diluted to initiate off-exchange, and injected into a flowing stream of physiologic aqueous solvent flowing over a column of support matrix consisting of avidin covalently bound to the matrix. The avidin utilized may variously consist of streptavidin, egg white avidin, or monomeric avidin, or other modified forms of avidin. The linkage to matrix may be way of any of a variety of functionalities including sodium cyanoborohydride-stabilized Shiff base or that resulting from the cyanogen bromide procedure as applied to carbohydrate matrices. The solid matrices may consist of cross-linked agarose particles or preferably perfusive supports such as those (Poros products) provided by the Perceptive Biosystems company (solid support 20-AL and the like).

For many binding pairs off-exchange may terminated and selective elution of binding protein accomplished by simply shifting pH to about 2.2 at 0° C. These conditions disrupt many types of binding protein-binding partner complexes but do not disrupt the avidin-biotin interaction, thereby allowing retention on the column of biotinylated binding partner. If shifting to acidic conditions by itself does not result in elution of a particular binding protein, then one of a variety of additional denaturants can be added to the elution solvent, including urea, guanidine hydrochloride, and guanidine thiocyanate at concentrations (preferably 2-4 M guanidine hydrochloride, 1-2 M guanidine thiocyanate) sufficient to elute binding protein but not at the same time disrupt the avidin-biotin interaction and thereby co-elute the binding partner. In general, these conditions do not disrupt the avidin-biotin interaction, even at room temperature. Finally, as above, reductants, such as TCEP can optionally be admixed with the elution solvent so that it will be present in the binding partner sample when desired.

An additional advantage of the support matrix approach to exchange reactions is that certain embodiments require that the binding protein and binding partner of interest be on-exchanged, complexed with each other, and off-exchanged while present within a mixture of other proteins and biomolecules. In these embodiments, as off-exchange proceeds, it is necessary to isolate the specific binding pair complex of interest. In a preferred embodiment this is accomplished with support matrices as follows. Previously biotinylated binding partner is contacted with a sample containing a mixture of proteins, perhaps a suspension of intact, living cells, or a whole cell extract or digest, or a biologic fluid, such as serum, plasma or blood that also contains the binding protein of interest. Said contacting and mixing results in formation of the biotinylated binding partner-binding protein complex. This mixture, of which the binding pair may be a minor component, is then passed over the aforementioned support matrix containing avidin, wherein the biotinylated complex of interest will specifically attach to the matrix. Washing of the support with aqueous solvent continues (or when desired may initiate) off exchange and removes from the matrix the irrelevant proteins that were present in the initial mixture, and thereby purifies the binding protein-binding partner complex. At the end of the off-exchange interval, the purified binding protein is simultaneously eluted and shifted to slow exchange conditions as above with an aliquot of appropriate eluent.

Optionally, antecedent detergent or otherwise mediated solubilization or extraction of membrane-bound proteins can be performed, and clarification of particulate material rapidly accomplished by filtration, or through use of slurries of magnetic support matrix particles (for example Dyna-Beads), as long as these procedures are performed in a manner that does not dissociate the binding protein-biotinylated binding partner pair complex.

One of the virtues of the affinity chromatography aspects of this invention is that binding-incompetent (and likely structurally and functionally incompetent) portions of the proteins under study are removed from the sample under study before it proceeds to further proteolysis and analysis. The affinity chromatography inherent in the instant invention removes the functionally altered portions of the target protein from the analytical stream.

As an alternative to the dilution of on-exchanged protein solutions to achieve the initiation of off-exchange reactions, microfluidic devices may be employed. Initiation of off-exchange by substantial dilution of on-exchanged binding protein compromises both kinetic and equilibrium properties of the binding protein-binding partner interaction that are important for optimal hydrogen exchange study of binding interactions. First, it is important that complexes between binding protein and binding partner are formed as rapidly as possible (fast association time) and it is also important that the two proteins remain associated with each other as often as possible during the off-exchange. Both the kinetics of initial complex formation and equilibrium binding are dramatically affected by the concentrations of the binding protein and binding partner. The higher the concentrations of the two, the faster complexes will be formed between them, and the higher the concentration of the complex will be at equilibrium. When off-exchange is initiated by dilution of binding partner, this necessarily decreases the final concentrations of binding protein and binding partner with a resulting decrease in the rate of initial complex formation and the equilibrium concentration of complex. Additionally, dilution of the binding protein necessarily results in proportionate increases in the volume of the resulting off-exchange solution that is to be further processed. The result is that substantial limitations are placed on the amount of binding protein solution that can be utilized in each sample, as it must subsequently be fragmented on an endopeptidase column with limits on sample volume capacity.

Moreover, certain target proteins require lipid or detergent environments for expression of their physiologic structure and function. Slowed-exchange-compatible proteolysis of such protein targets can be accomplished with current methods, but further analyses (c18 reversed-phase chromatography, ESI-MS) are not possible because of interference from the associated lipids and/or detergents. The use of microfluidic devices allows such interfering substances to be efficiently and rapidly separated from the peptide fragments, allowing their effective analysis, for example using deuterium exchange-mass spectrometry (DXMS).

Through the use of microfluidic devices, solutions containing target proteins have their buffer composition changed by allowing effective diffusion of the smaller buffer components (²H₂O, H₂O, salts, ligands) without effective diffusion of the target protein. In one embodiment, small regenerated cellulose microdialysis fibers (13,000 or 18,000 MWCO, approximately 200 u ID; Spectrum Inc.) are encased in PEEK tubing ( 15/1000 inch ID) with endfittings that allow a countercurrent sheath solvent flow of exchange solvent while the protein solution flows through the microdialysis fiber. Such devices are capable of very efficient ²H₂O exchange in short times, for example, effecting change to 95% 2H₂O in three seconds at room temperature. Typical flow rates to achieve this end consist of 50 μl/minute for protein solution and 1000 μl/minute for sheath solution.

Such microfluidic devices can also be used to semipurify peptide mixtures that are contaminated with interfering lipids and detergents, such as proteolytic digests of membrane protein preparations. In this application, the proteolytic digest of such a protein is passed through the bore of the microdialysis fiber (flow 5-50 μl/minute) while the countercurrent sheath flow (100-400 μl/min), into which peptide fragments can transfer, (but not the more slowly diffusing and non-dialyzable lipid/detergent micelles), is directed to and collected on the c18 column for collection and subsequent acetonitrile-gradient elution and MS The result is that the digest peptides can be analyzed without interference from the lipid/detergent

Non-constrained devices which utilize differential diffusion to effect changes in buffer composition (such as the “H-reactor” patented by Micronics, Inc.) can also be employed for these purposes. With these devices, flow of sample and exchange buffer is concurrent, not countercurrent, and exchange is therefore necessarily less efficient for a given volume of exchange buffer employed.

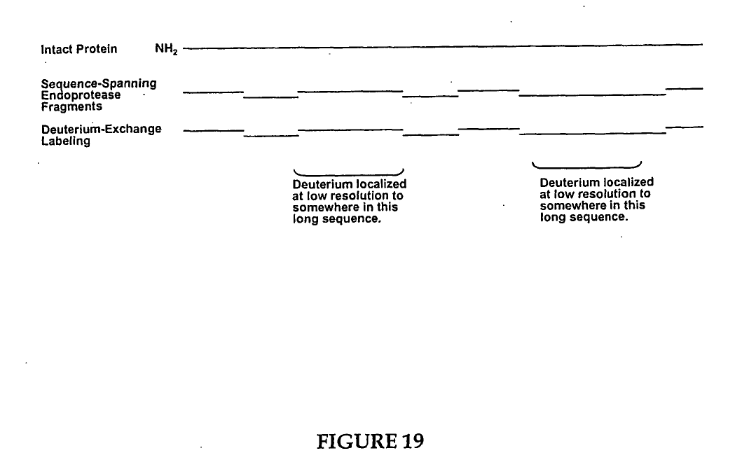

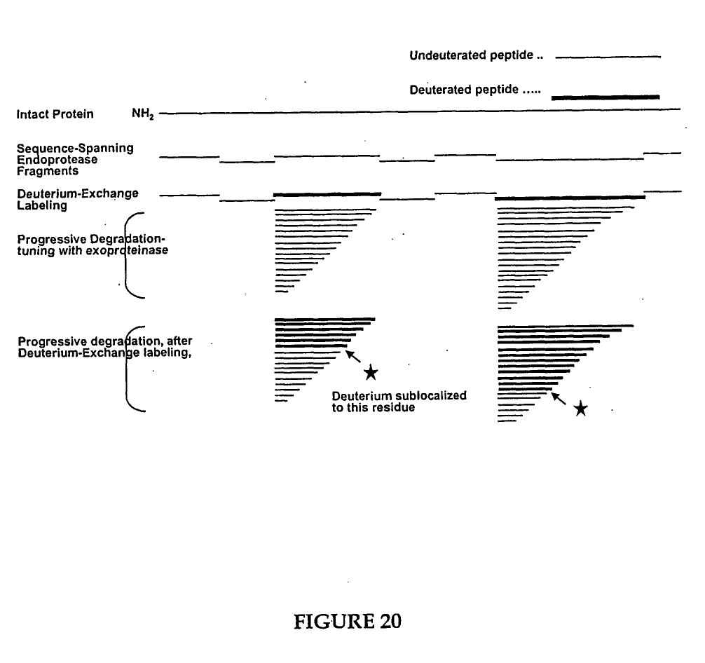

According to the methods of the present invention, it was surprisingly determined that the optimization of a number of experimental parameters relevant to endopeptidase fragmentation, together with refined analysis of the deuterium content of the resulting fragments, enabled the use of simple endopeptidase digestion to generate a densely sequence-overlapping population of peptides for analysis. Prior teachings had found that the common acid-resistant endopeptidases alone, such as pepsin, were not useful in highly localizing amide hydrogen exchange due to insufficient ability to fragment target proteins under acceptable slowed exchange conditions. Pepsin, as employed in the prior art typically had generated a relatively small number of fragments, generally 10-25 amino acids long. The label incorporated on these few useable pepsin-generated peptides was then used to infer the location of label, at best localizing within a range of about 10-25 amino acids (see FIG. 19). Subsequent art taught the use of acid-resistant carboxypeptidases (progressive degradation) after an initial employment of endopeptidases, to localize the labeled amino acid positions within peptides generated when a detailed resolution, such as within 1-5 amino acid residues, is desired (see FIG. 20).

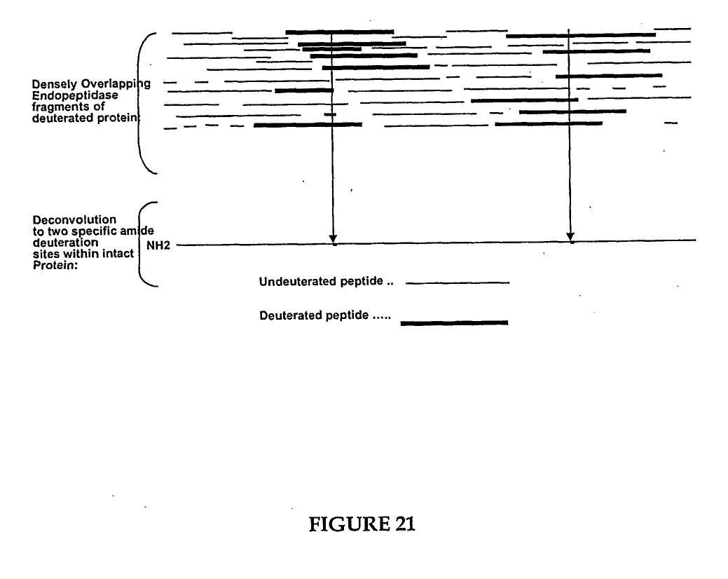

In the present invention, improved methods that dramatically speed proteolysis, and modulate the sites and patterns of proteolysis proteolysis by endoproteinases are employed so as to produce highly varied and highly efficient fragmentation of the labeled protein in a single step, thereby avoiding the use of carboxypeptidases completely, an improvement which simplifies the fragmentation and affords a considerable savings of time and cost. (See FIG. 21). While these improvements work best in combination with each other, they can be grouped into 3 categories: (i) use of denaturants (systematically varying the type, concentration, duration of denaturation, type of endoproteinase(s) employed, and the duration of endopeptidase digestion) to greatly speed proteolysis and modulate the resulting pattern of fragmentation; (ii) use of solid-state proteolysis with acid-resistant endopeptidases selected for their efficiency and distinctive fragmentation preferences with respect to each other under optimal quench conditions; and (iii) use of water-soluble phosphines to effect rapid and efficient disulfide reduction under quench conditions

The use of such endopeptidases under optimized conditions described herein routinely results in the generation of a population of endopeptidase fragments substantially spanning the full length of the majority of proteins studied to date, and, as importantly, yields a large number of additional peptides that partially and mutually overlap in sequence with each other, all obtainable in useful yield. Preferably, the population of fragments contains sequence-overlapping fragments wherein more than half, more preferably 60%-80%, of the members of the population have sequences that are overlapped by the sequences of other members by all but 1-5 amino acid residues. In addition, it is preferable that a majority of members of the population of fragments is present in an analytically sufficient quantity to permit its further characterization, for example, by LC-MS analysis.

With some proteins, there is an absolute requirement for the employment of denaturants to effect fragmentation under quench conditions. An example of a protein with such an absolute dependency is Hen Egg White Lysozyme (HEL). In a preferred embodiment, the labeled protein is exposed, before fragmentation, to denaturing conditions compatible with slow hydrogen exchange and sufficiently strong to denature the protein enough to render it adequately susceptible to the intended proteolytic treatment. If these denaturing conditions would also denature the protease, then, prior to proteolysis, the denatured protein is switched to less denatured conditions (still compatible with slow H-exchange) sufficiently denaturing to maintain the protein in a protease-susceptible state but substantially less harmful to the protease in question. Preferably, the initial denaturant is guanidine thiocyanate, and the less denaturing condition is obtained by dilution with guanidine hydrochloride. Guanidine hydrochloride is an effective denaturant at a concentration of about 0.05-4 M.

In previous studies by Englander et al. and others recited above, proteolytic fragmentation of labeled proteins under slowed-exchange conditions was suitably accomplished by simply shifting the protein's pH to 2.7, adding high concentrations of liquid phase pepsin, followed by (10 minute) incubation at 0° C. With the proteins studied and reported by others to date, simply shifting pH from that of physiologic (7.0) to 2.7 was sufficient to render them sufficiently denatured as to be susceptible to pepsin proteolysis at 0° C. Furthermore, these reported proteins, in general, did not contain disulfide bonds that interfered with effective denaturation by such (acid) pH conditions or contain disulfide bonds within portions of the protein under study with the technique.

However, the applicant has found that other proteins (for example, HEL) are negligibly denatured and are not substantially susceptible to pepsin proteolysis when continuously incubated at comparable acidic pH and depressed temperature (10-0° C.) for several hours. This is likely the consequence of the existence of a thermal barrier to denaturation for many proteins incubated in many denaturants; i.e., denaturation of proteins at lower temperatures (10-0° C.), an absolute requirement for hydrogen exchange quench, is often inefficient and a slow process, incompatible with the requirement of medium resolution hydrogen exchange techniques that manipulations be performed rapidly, such that the attached label is substantially retained at functionally labeled amides of the protein.

It is disclosed herein that such proteins become extraordinarily susceptible to pepsin proteolysis at 0° C. when they are treated with the sequential denaturation procedure described below.

While proteins are often subjected to purposeful denaturation with agents other than a pH shift prior to digestion with pepsin, this has never been done at depressed temperatures (10-0° C.) before, and it is disclosed herein that while guanidine thiocyanate at the indicated concentrations is sufficient to suitably denature and render susceptible to pepsin proteolysis proteins at 10-0° C., several other strong denaturants, including urea, HCl, sodium dodecyl sulfate (SDS) and guanidine HCl, were, at least when used alone, unable to adequately denature lysozyme at these low temperatures. However, the concentrations of guanidine thiocyanate required for such denaturation are incompatible with pepsin digestion; i.e., they denature the pepsin enzyme before it can act on the denatured binding protein. When the guanidine thiocyanate is removed (at 10-0° C.) from the solution after protein denaturation has been accomplished in an attempt to overcome this inhibition of pepsin activity, the protein rapidly refolds and/or aggregates, which renders it again refractory to the proteolytic action of pepsin.

It is further disclosed herein that if proteins are first denatured in about 1.5-4 M (preferably ≧2M) guanidine thiocyanate at 0° C. and the concentration of thiocyanate then reduced to preferably ≦0.5 M, while at the same time the guanidine ion is maintained at about 0.05-4 M (preferably ≧2M) (by diluting the guanidine thiocyanate-protein mixture into guanidine hydrochloride solution), the denatured protein remains in solution, remains denatured, and the enzyme pepsin remains proteolytically active against the denatured protein in this solution at 0° C. The denatured (or denatured and reduced) protein solution is then passed over a pepsin-solid-support column, resulting in efficient and rapid fragmentation of the protein (in less than 1 minute). The fragments can be, and usually are, immediately analyzed on RP-HPLC without unnecessary contamination of the peptide mixture with the enzyme pepsin or fragments of the enzyme pepsin. Such contamination is problematic with the technique as taught by Englander et al., as high concentrations of pepsin (often equal in mass to the protein under study) are employed, to force the proteolysis to occur sufficiently rapidly at 0° C.

The stability of pepsin-agarose to this digestion buffer is such that no detectable degradation in the performance of the pepsin column employed by the applicant has occurred after being used to proteolyze more than 500 samples over 1 year. No pepsin autodigestion takes place under these conditions. Denaturation without concomitant reduction of the binding protein may be accomplished by contacting it (at 0-5° C.) with a solution containing ≧2M guanidine thiocyanate (pH 2.7), followed by the addition of an equal volume of 4 M guanidine hydrochloride (pH 2.7).

Subsequent to the discovery herein of the extraordinary stability to denaturation of HEL under quench conditions, and the foregoing remedy, it has been found that all other proteins studied to date by the applicant are susceptible, at least to a minimal degree, to pepsin proteolysis under simple quench conditions, but that the speed and extent of fragmentation can be dramatically increased by the addition of suitable concentrations of guanidine hydrochloride (GuHCl) alone, without the use of guanidine thiocyanate. There is considerable virtue in avoiding the use of thiocyanate when possible: there is a variable (often severe) aggregation and precipitation of some of the denatured protein as the thiocyanate is diluted out prior to proteolysis, greatly confounded automated sample processing.

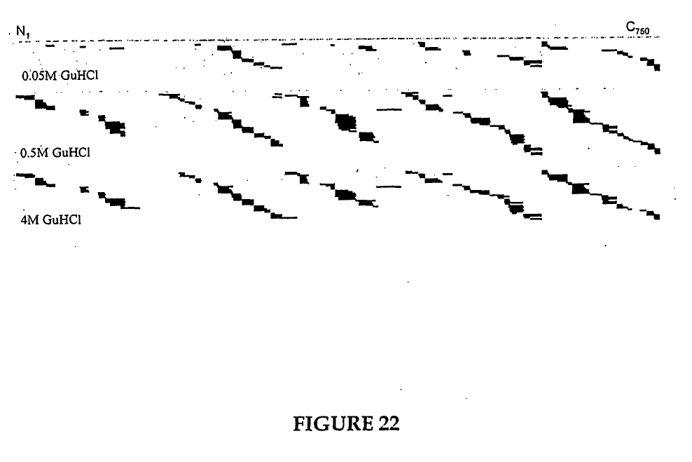

FIG. 22 presents the fragmentation patterns seen when the protein phospholipase A2, under quench conditions (pH 2.2, 0° C.), is incubated with varying concentrations of GuHCl (0-4 M) for 20 seconds, and then contacted with a pepsin-solid support column (30 mg/ml, Poros 20-AL media; 66 microliters bed volume) for an additional 30 seconds. Fragments were contemporaneously loaded on a microbore c18 column and then eluted with an acetonitrile gradient over 10 minutes, again at pH 2.2, 0 degrees C., with the effluent directed to a Finnigan LCQ mass spectrometer operating at a capillary temperature of 200° C. Data was acquired in both data dependent MS2 mode (to allow peptide identification) and in MS1 mode (to assess the suitability of the isotopic envelope data for subsequent deuterium quantification (signal, signal to noise, spectral overlap with confounding fragments). When no GuHCl was used, less than 15 peptides in sufficient yield were identified (data not shown). When 0.05 M GuHCl was used, more than 70 high quality peptides were obtained, with further improvement when 0.5 M denaturant was employed. While 4.0 M denaturant resulted in the identification of additional peptides, some were also lost when compared to the 0.5 M digest.

Several variables have been found herein to behave independently in determining the speed and pattern of digestion, and that their effects are distinctive for each target protein studied. Typically, up to 30 combinations of these variables (employing the automated features of the apparatus described herein) are evaluated to establish optimal fragmentation conditions for the protein under study. These independent variables consist of the type of denaturant (GuSCN versus GuHCl); its concentration preferably (0.05-4 M); the time the denaturant is allowed to act on the protein prior to fragmentation (preferably 0 to 3 minutes); the type(s) of endoproteinases employed; and the time allowed for digestion (preferably 20 seconds to 2 minutes). For most proteins studied, GuHCl, at a concentration of 0.5 M and 30 seconds fragmentation on a pepsin column as above is near optimal, though more extensive tuning will always improve the fragmentation map.

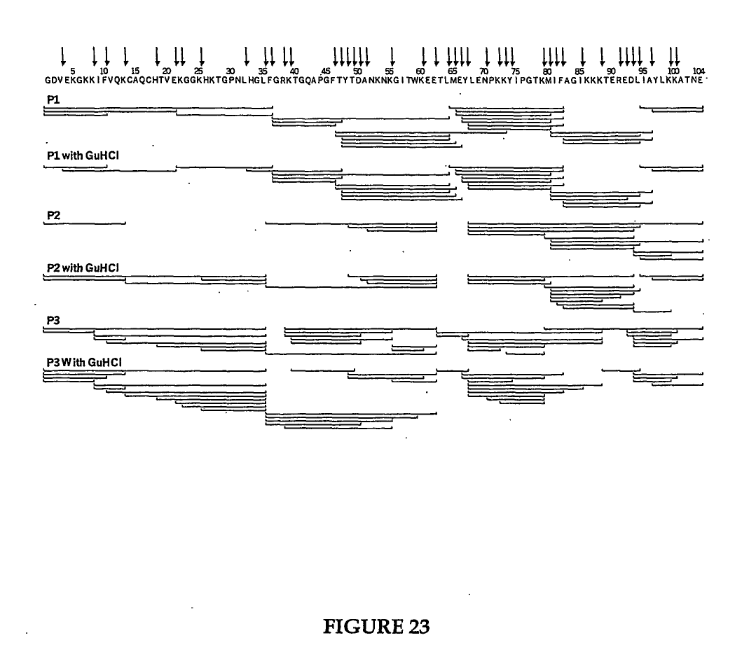

FIG. 23 presents results obtained when horse cytochrome c was quenched with or without 0.5 M GuHCl, and then fragmented with either pepsin (P1), Newlase (P2) or Fungal Protease XIII (P3) coupled to perfusive supports (20-30 mg/ml), and analyzed as in FIG. 22. Considerable variation in digestion pattern and yield is seen with the several conditions. The arrows at the top of the figure indicate the positions of the C- and N-termini of the aggregate peptides produced, highlighting the extreme degree of overlap of the resulting set of peptide fragments. Even when denaturant is omitted the results of the solid state pepsin digestions were superior to those disclosed by others in which cytochrome c was fragmented with liquid-phase pepsin, where fewer than 15 useful peptides were obtained in a 10 minute digestion.

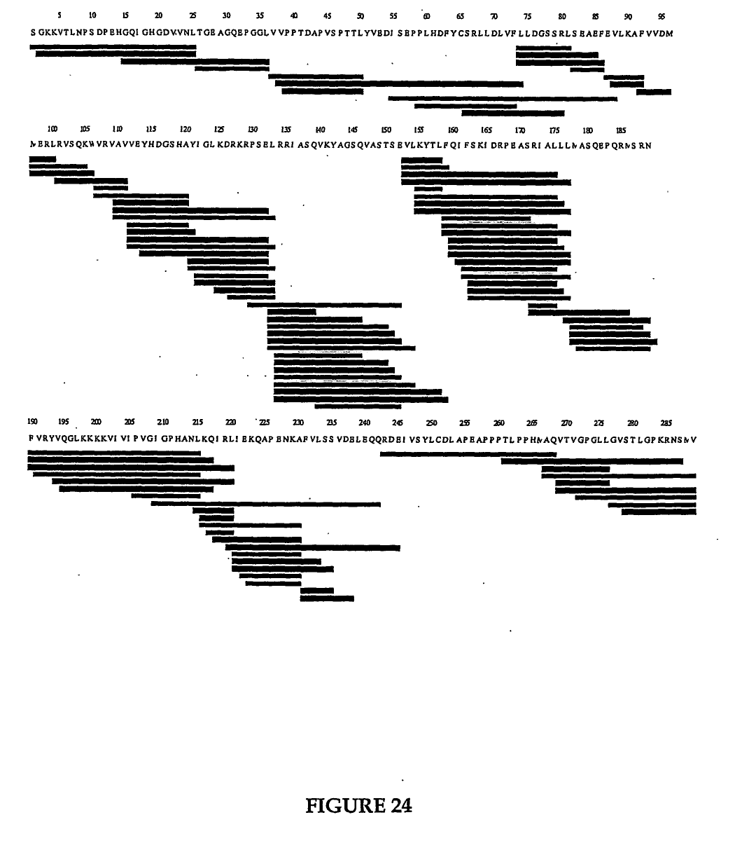

FIG. 24 presents the fragmentation map obtained for a human von Willebrand Factor construct (denatured in 0.5 M GuHCl) employing a 40 second digestion on a pepsin column. In this study it was necessary to simultaneously reduce an internal disulfide bond by mixing TCEP (1.0 M final concentration) with the denaturant

It is to be emphasized that the speed of generation (typically in 30 seconds) and the yield and extent of the highly overlapping fragmentation seen using the methods of the present invention is unprecedented in the previously disclosed art, and was unanticipated, until these recent results described herein. There was no expectation that the art of modulating endoprotease activity-both in terms of producing the needed varied fragmentation and yield could be enhanced enough to be useful by itself for high resolution localization of label. Heavy hydrogen label is quickly lost from proteolytic fragments during analysis, even under quench conditions: thus, all steps of analysis should be performed as quickly as possible, including protease digestion. The methods developed and available prior to 1997 required pepsin degradation durations that were already at the upper limits of acceptable times (approximately 10 minutes). For example in U.S. Pat. No. 6,291,189, it is stated that: “In a preferred embodiment, pepsin is used, preferably at a concentration of 10 mg/ml pepsin at 0° C. pH 2.7 for 5-30 minutes, preferably 10 minutes.” It was therefore unanticipated that more extensive digestions could be obtained with pepsin with or without other endoproteinases given the time constraints of amide hydrogen exchange study. Indeed, the perceived shortcomings of endoproteinases, now overcome, were the principal motivations for the development and use of progressive degradation with exoproteases (carboxypeptidases) to achieve higher resolution localization of label.

Accordingly, the methods of the present invention analyze endopeptidase fragments that are generated by cleaving the labeled protein with an endopeptidase selected from the group consisting of a serine endopeptidase, a cysteine endopeptidase, an aspartic endopeptidase, a metalloendopeptidase, and a threonine endopeptidase (a classification of endopeptidases by catalytic type is available on the world wide web at the URL “chem.qmul.ac.uk/iubmb/enzyme/EC34”; by the Nomenclature Committee of the International Union of Biochemistry and Molecular Biology). Presently preferred endopeptidases include pepsin, newlase and acid tolerant Aspergillus proteases such as Aspergillus protease XIII. FIG. 23 demonstrates the distinctive fragmentation patterns that can be obtained with each of the endoproteinases the applicant employs. It has further been found that the fragmentation patterns resulting from simultaneous, and/or sequential proteolysis by combinations of these enzymes are additive in their effect on fragmentation. Therefore, more than one endopeptidase may be used in combination. Optimally, endopeptidase fragments are generated at a pH of about 1.8-3.4, preferably 2-3, more preferably in the range of about 2.1-2.3 or 2.5-3.0.

In preferred embodiments, the endopeptidase may be coupled to a perfusive support material to facilitate manipulation of digestions, as an alternative to liquid phase digestions. This allows the reuse of endopeptidase materials and separates the enzyme from the fragments for further analysis. Exemplary perfusive support matrices include Poros 20 media, wherein digestion of the labeled protein is accomplished by contacting a solution of the labeled protein with said matrix, followed by elution of generated fragments from the matrix. With the use of the solid support, sample digestion under slowed exchange conditions can be performed that results in no detectable endoproteinase autodigestive fragments being released into the digestion product, i.e., the population of labeled protein fragments. Furthermore, the endoproteinases remain fully active and available for subsequent repeated use as a digestive medium for additional samples.

In has been discovered herein that the judicious admixture of denaturants with substrate protein results in the ability to greatly promote and “tune” substrate fragmentation and this discovery was first made employing liquid-phase forms of the proteases. Unfortunately, these same denaturants retard and/or inhibit the activity of the enzymes unless denaturants are partially removed prior to proteolysis. However their removal allows the substrates to re-fold, negating the benefit of the denaturant. Gradual manual dilution of the substrate-enzyme-denaturant mixture allowed an initially slow proteolysis to proceed, and with subsequent dilution, partially degraded substrate is unable to refold; and because of denaturant dilution, protease activity increases, further fragmenting the initial large substrate fragments. Success in this method required multiple manual additions of reagents, denaturants, and timed addition of diluents, all very labor intensive. The improved methods of the present invention use solid-state enzymes on perfusive supports and column chromatography, enabling samples to be applied to the column already mixed with denaturant, and the necessary dilution of denaturant automatically occurs as the substrate slug passes down the column, now progressively diluted with the fluid in the column void volume as proteolysis proceeds. This results in tremendous labor savings, and is readily automated. There is thus an unanticipated ease and simplification of use of the necessary denaturants when solid phase proteases are employed.

A variety of acid-reactive endoproteinases can be covalently coupled to any of a number of available support matrices including, for example, cross-linked dextran, cross-linked agarose, as well as more specialized supports suitable for modern HPLC chromatography, preferably the Poros line of perfusive support materials supplied by Perceptive Biosystems, such as “20-AL” and the like. These latter supports are particularly advantageous for invention methods as they allow rapid interaction of substrate with bound peptidases. The coupling of endoproteinases to matrices can be achieved by any of a number of well-known chemistries capable of effecting such couplings, including, for example, aldehyde-mediated (sodium cyanoborohydride-stabilized Shiff base), carboiimide, and cyanogen bromide-activated couplings. Conditions, including pH, conducive to the continued stability of particular peptidases may optionally need to be employed, and could readily be implemented by one of skill in the art.

An exemplary preparation of coupled endopeptidase is as follows. The endopeptidase is obtained as a lyophilized powder, reconstituted with distilled water, and dialyzed against a coupling buffer consisting of 50 mM citrate (pH 4.4). The peptidase is then coupled to Perceptive Biosystems Poros media 20-AL following the manufacturer's recommending coupling procedures, including “salting out” with high sodium sulfate concentrations. Couplings can be performed at a ratio of 5 to 30 mg of peptidase per ml of settled 20-AL matrix, preferably 30 mg/ml. The coupled matrix can then be stored in the presence of sodium azide to minimize bacterial contamination.

While any of a number of batchwise or column chromatographic approaches might be employed to effect matrix-bound endopeptidase digestion of labeled protein under slowed exchange conditions, we have found the following approach to work well and to be preferable. A stainless steel column (length 2 cm, width 2.2 mm, internal volume approximately 66 microliters) was packed with endoproteinase-derivatized 20-AL support coupled with protein at 30 mg/ml) and flow established with a solvent consisting of 0.5% formic acid (for pepsin, newlase, or Aspergillus protease XIII), said column being operated at 0° C. Care must be taken to employ buffers with a pH compatible with rapid peptidase action: buffers with a pH of 2.7-3.0 (room temperature measurement) work well. An aliquot of labeled protein to be fragmented was contacted with the column matrix typically in a volume of 10-300 microliters, preferably 100 microliters, and the sample allowed to reside on the column for a time determined (by preliminary titration studies) to result in the desired degree of fragmentation. The Applicant has surprisingly found that digestion times of 13 seconds to 5 minutes, preferably less than a minute, more preferably, less than 40 seconds to be optimal. Prior knowledge of endopeptidase digestion suggested that digestion times of greater than 10 minutes would be required to produce sufficient fragmentation. The sample was then flushed from the column onto either an analytical reverse phase HPLC column for subsequent separation and analysis of the peptide fragments, or directly without additional purification or chromatography onto a mass spectrometer for analysis. During this analysis period, the column is flushed (with the effluent going to waste) with an excess of solvent to remove any peptide or subfragments which nonspecifically adhere or are otherwise retained in the matrix, thereby preparing the column for a repeated use. Such washing buffers can be any of a wide variety of buffers including the buffers used for digestion. The column-washing step (between each sample digestion) is preferable but not absolutely required for success.

In an additional embodiment, a column containing one of these solid state proteases can be used to further digest peptides on-line as they each independently exit the reversed phase (rp) HPLC column during gradient elution. This approach has the considerable advantage of producing a much less complex mixture of peptides to analyze than when two enzymes act on the substrate before rpHPLC. To use these enzymes in this post-chromatography manner, we have found it necessary to reduce the acetonitrile concentration in the effluent stream prior to passage over the protease column, as acetonitrile can reversibly (and irreversibly) inhibit these enzymes.

In addition, disulfide bonds, if present in the protein to be digested, can also interfere with analysis. Disulfide bonds can hold the protein in a folded state where only a relatively small number of peptide bonds are exposed to proteolytic attack. Even if some peptide bonds are cleaved, failing to disrupt the disulfide bonds would reduce resolution of the peptide fragments still joined to each other by the disulfide bond; instead of being separated, they would remain together. This would reduce the resolution by at least a factor of two (possibly more, depending on the relationship of disulfide bond topology to peptide cleavage sites).

In one embodiment, water-soluble phosphines, for example, Tris(2-carboxyethyl)phosphine (TCEP) may be used to disrupt a protein's disulfide bonds under “slow hydrogen exchange” conditions. This allows much more effective fragmentation of large proteins which contain disulfide bonds without causing label to be lost from the protein or its proteolytic fragments (as would be the case with conventional disulfide reduction techniques which must be performed at pHs which are very unfavorable for preservation of label).

High resolution localization of label-bearing amides with the use of endoproteinases requires the proteolytic generation of numerous sequence-overlapped under conditions that allow the label to remain in place (e.g., 0° C., pH 2.2). The ability of any protease to fragment a protein or peptide is limited by the accessibility of the protease to susceptible peptide bonds. While denaturants such as acidic pH, urea, detergents, and organic co-solvents can partially denature proteins and expose many otherwise structurally shielded peptide bonds, pre-existing disulfide bonds within a protein can prevent sufficient denaturation with these agents alone. In conventional protein structural studies, disulfides are usually cleaved by reduction with 2-mercaptoethanol, dithiothreitol, and other reductants which unfortunately require a pH greater than 6 and elevated temperature for sufficient activity, and are therefore not useful for the reduction of disulfides at pH 2.7 or below. For this reason, the hydrogen exchange art has not attempted any form of disulfide bond disruption, has for the most part been restricted to the study of proteins without intrinsic disulfide bonds, and has accepted the low resolution achievable without disulfide bond disruption.

It is demonstrated herein that acid-reactive phosphines such as Tris(2-carboxyethyl)phosphine (TCEP) can be used to disrupt disulfides under the acidic pH and low temperature constraints required for hydrogen exchange analysis. These manipulations disrupt these associations and at the same time continue to produce a markedly slowed proton exchange rate for peptide amide protons.

Denatured (with or without reduction) labeled protein is then passed over a column composed of insoluble (solid state) pepsin, whereby during the course of the passage of such denatured or denatured and reduced binding protein through the column, it is substantially completely fragmented by the pepsin to peptides of size range 2-20 amino acids at 0° C. and at pH 2.7. The effluent from this column (containing proteolytically-generated fragments of labeled protein) is directly and immediately applied to the chromatographic procedure employed to separate and analyze protein fragments, preferably analytical reverse-phase HPLC chromatography and/or mass spectrometry.

In preferred embodiments, proteins containing disulfide bonds may be first physically attached to solid support matrices, and then contacted with solutions containing TCEP at acidic pH and low temperature for more rapid reactions than are possible in solution. In this preferred embodiment, with all steps performed at 5-0° C., preferably 0° C., the protein in aqueous solution, with or without prior denaturation and under a wide variety of pH conditions (pH 2.0-9.0) is first contacted with a particulate silica-based reverse-phase support material or matrix typically used to pack HPLC columns, including C4 and C18 reversed phase silica supports, thereby attaching the protein to the surface of such material. Unbound binding protein may then optionally be washed off the support matrix with typical aqueous HPLC solvents, (0.1% trifluoroacetic acid, (TFA) or 0.1-0.5% formic acid in water, buffer A). An aliquot of a substantially aqueous buffer containing TCEP at a pH between 2.5 and 3.5, preferably 2.7 is then contacted with the protein that is attached to the support material and allowed to incubate with the attached protein near 0° C. and preferably for short periods of time (0.5-20 minutes, preferably 5 minutes) and then the TCEP-containing buffer removed from the support matrix by washing with buffer A, followed by elution of the reduced binding protein from the support matrix by contacting the support with eluting agents capable of disrupting the support-protein binding interaction, but also compatible with continued slow hydrogen exchange (pH 2.0-3.5; temperature 0-5° C.).

An example of this preferred embodiment to achieve disulfide reduction prior to pepsin fragmentation is as follows. Labeled protein is applied to a reverse phase silica-based C18 HPLC support matrix in a column (for example, Vydac silica-based C18, catalog #218TP54, or Phenominex silica-based C18 Jupiter 00B4053-B-J) that has been pre-equilibrated with HPLC solvent “A” (0.1% TFA or 0.1-0.5% formic acid at 0-5° C. After substantial binding of the lysozyme has occurred (usually within seconds), additional buffer A is passed through the column to remove small quantities of unattached binding protein. A solution containing TCEP (50-200 micrometers of TCEP (0.05-2.0 M in water at a pH of 2.5-3.5, preferably 3.0) is then applied to the column in a manner that results in its saturation of the portion of the column to which the binding protein has been previously attached. Flow of solvent on the support is then stopped to allow incubation of the TCEP solution with the support matrix-attached binding protein. At the end of this incubation time (variously 0.5 minutes-20 minutes, preferably 5 minutes) flow of buffer A is resumed, resulting in the clearance and washing of the TCEP solution from the support matrix. This is followed by application of an amount of buffer B (20% water, 80% acetonitrile, 0.1% TFA) sufficient to release the binding protein from the support (typically 30-50% solvent B in solvent A). This eluted and reduced protein is then passed over a pepsin column to effect its fragmentation under slowed exchange conditions. The protein fragments resulting from the action of the pepsin column on the reduced protein are then contacted with another analytical HPLC column, preferably a reverse phase HPLC support, and the fragments sequentially eluted from the support with a gradient of solvent B in solvent A.

An example of an alternative preferred embodiment to achieve disulfide reduction after pepsin fragmentation is as follows. This alternative approach is to first denature the protein under slow exchange conditions, pass it over a pepsin column to effect fragmentation, apply the resulting fragments to a HPLC support matrix, effect reduction of the support-bound peptide fragments by contacting them with the aforementioned TCEP solution, followed by sufficient incubation at 0° C., finally followed by elution of the reduced fragments from the column with increasing concentrations of solvent B. The advantage of this second alternative method is that an entire HPLC support matrix attachment-detachment step is avoided, resulting in a simplification of the manipulations and equipment required for the procedure, as well as savings in elapsed time. This approach will not work when a particular protein requires substantial prior reduction of disulfides to become substantially susceptible to the digestive actions of pepsin. Certain proteins are sufficiently stabilized by their contained disulfide bonds that they do not become substantially susceptible to pepsin even in the presence of strong denaturants. In such cases it will be preferable to apply the first method of reduction (above), where the protein is first reduced “on column”, eluted, fragmented on the pepsin column, and the fragments then optionally applied to an additional column matrix to effect separation from each other.

Additionally, it is disclosed herein that the simultaneous use of denaturants and reductants (TCEP) results in synergistic enhancement of both protein denaturation and reduction, not afforded when employed separately, or even sequentially.

Mass spectroscopy has become a standard technology by which the amino acid sequence of proteolytically generated peptides can be rapidly determined. It is commonly used to study peptides that contain amino acids that have been deuterated at carbon-hydrogen positions, and thereby determine the precise location of the deuterated amino acid within the peptide's primary sequence. This is possible because mass spectroscopic techniques can detect the slight increase in a particular amino acid's molecular weight due to the heavier mass of deuterium. McCloskey (Meth. Enzymol. 193:329-338, 1990) discloses use of deuterium exchange of proteins to study conformational changes by mass spectrometry. The methods of the present invention include measuring the mass of endopeptidase fragments to determine the presence or absence, and/or the quantity of deuterium on the endopeptidase fragments. Preferably, mass spectrometry is used for mass determination of these peptide fragments. This allows determination of the quantity of labeled peptide amides on any peptide fragment.

According to the methods of the present invention, proteolytically generated fragments of protein functionally labeled with deuterium may be identified, isolated, and then subjected to mass spectroscopy under conditions in which the deuterium remains in place on the functionally labeled peptide amides. Standard peptide sequence analysis mass spectroscopy can be performed under conditions which minimize peptide amide proton exchange: samples can be maintained at 4° C. to 0° C. with the use of a refrigerated sample introduction probe; samples can be introduced in buffers which range in pH between 1 and 3; and analyses are completed in a matter of minutes. MS ions may be made by MALDI (matrix-assisted laser desorption ionization) electrospray, fast atom bombardment (FAB), etc. Fragments are separated by mass by, e.g., magnetic sector, quadropole, ion cyclotron, or time-of-flight methods. For MS methods generally, see Siuzdak, G., Mass Spectrometry for Biotechnology (Academic Press 1996).

Once the endopeptidase fragmentation data is acquired on functionally deuterated protein, it is then deconvoluted to determine the position of labeled peptide amides in an amino acid specific manner. In general, the term “deconvoluted” as used herein refers to the mapping of deuterium quantity and location information obtained from the fragmentation data onto the amino acid sequence of the labeled protein to ascertain the location of labeled peptide amides, and optionally their rates of exchange. Deconvolution may comprise comparing the quantity and/or rate of exchange of isotope(s) on a plurality of endopeptidase fragments with the quantity and rate of exchange of isotope(s) on at least one other endopeptidase fragment in the population of fragments generated, wherein said quantities are corrected for back-exchange in an amino acid sequence-specific manner. Labeled peptide amides can optionally be localized in an amino acid sequence-specific manner by measuring rates of off-exchange of functionally attached label under quenched conditions. The determination of the quantity and rate of exchange of peptide amide hydrogen(s) may be carried out contemporaneously with the generation of the population of endopeptidase fragments.

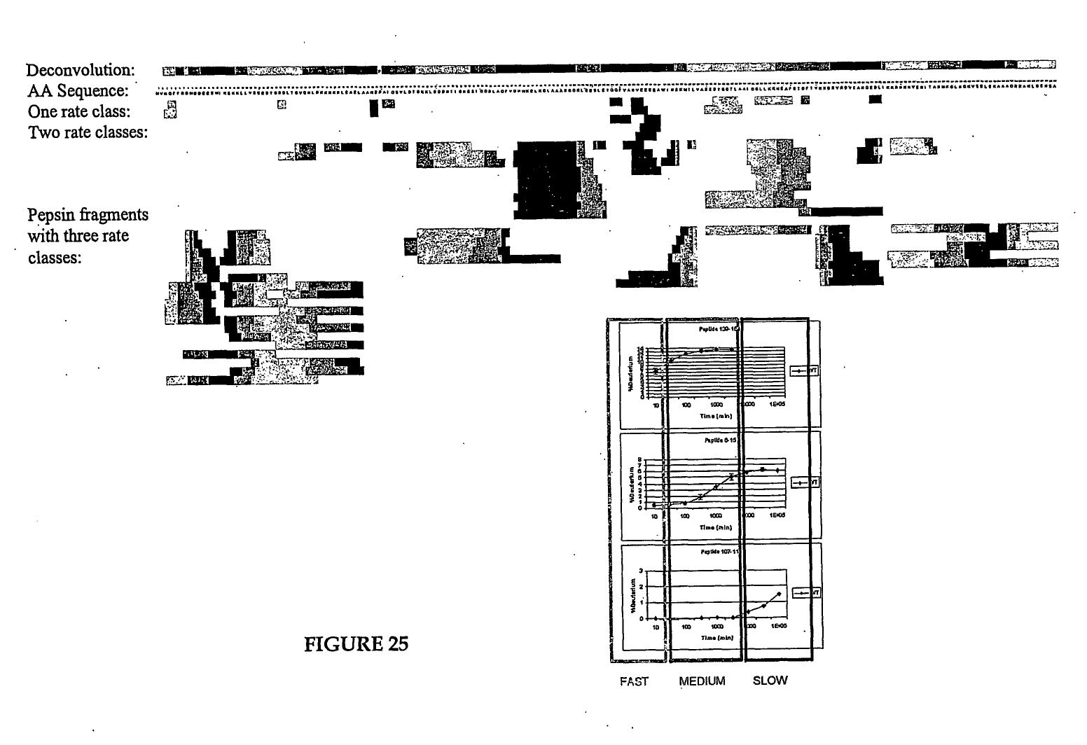

Although several alternative methods for effecting such deconvolution may be available, at least one useful method has been implemented and demonstrated herein. FIG. 25 presents the results of such a deconvolution. A protein construct composed of a two repeat segment (16-17) of chicken brain spectrin was on-exchanged in deuterated buffer for varying times (10 to 100,000 seconds, at 22° C.). Samples were then exchange-quenched, in 0.5 M GuHCl, pH 2.2, and otherwise processed as shown in FIG. 22. The deuterium content of the 113 useful peptides resulting from such fragmentation was determined from the raw MS data, with corrections for back-exchange made employing the inexact “peptide average” method of Zhang and Smith (Zhang et al., Prot. Sci. 2:522-531, 1993). Plots of deuterium buildup versus time were constructed for each peptide, and the number of amides exchanging in arbitrary “fast, medium and slow” classes (light, medium, and dark grey colors respectively in the figure) determined for each peptide. An initial map of rates versus amino acid sequence was then constructed from this information, employing a strategy in which “pieces” (fragments) with uniform rate class (color), were first placed in register, and subsequent placement of more complexly colored pieces (two colors then three colors) performed in a manner that required that the several “colors” in these peptides be reconciled vertically to conform with the color placement of the preceding pieces. The average color (rate class) at each amide position was then determined and used to construct the initial map. Unmeasurable amide hydrogens (approximately 10% of the total amides in the 113 fragments, unmeasured either because of errors incurred because of the approximate (average) back-exchange calculation method employed, or because the very slowest exchanging amides were not measured in this experiment) were then fit to the provisional map in a manner that minimized deviation from said map, and a final map constructed by averaging this final placement of “pieces”.

The choice of three rate classes was arbitrary, and done to simplify the “piece placement” work, which was done manually in this example. Assignment of amides in each peptide to each of 9 rate classes (9 time points were employed in this experiment) would considerably improve the resolution of the deconvolution, but is best performed by automated (computational) means, and with incorporation of more precise back-exchange corrections as discussed below. Further fragmentation of this protein construct with pepsin plus Fungal protease XIII has resulted in a 50% increase in the number of spectrin fragments, which will preferably be deconvoluted through linear programming-mediated approaches.

The essential attributes of a preferred deconvolution algorithm for such high density, overlapping endopeptidase fragment data include that: (i) it takes as inputs the measurements of the quantity of label on the numerous overlapping endopeptidase fragments correlated with their amino acid (aa) sequence; (ii) it more precisely corrects for back-exchange (that is, label lost subsequent to initiation of quench, during the analysis step) than the presently employed method that calculates an average correction factor for all amides in a peptide (Zhang et al., Prot. Sci. 2:522-531, 1993) and instead employs a correction that is sub-site-specific (specific for 1-5 contiguous amides, depending on the resolving power of the aggregate endoprotease fragments available). This can be done both computationally (by reference to the Bai/Englander algorithm; Bai et al., Proteins: Struct. Funct. Genet. 17:75-86, 1993) or alternatively experimentally by measuring back exchange, under quench conditions, of the substantially random coil fragments resulting from identical endoproteolysis of a fully (equilibrium) deuterated sample of the protein in a manner that allows the rate(s) of loss of deuterium to be measured over time for each resolvable sequence region. Both approaches afford precise calculation of the label lost through back exchange from each peptide, and, by comparison, that lost in each amino add segment that differs between amino acid sequence-overlapping peptides. Corrections for these losses are made for each peptide/aa overlap difference value; (iii) it compares the (corrected) label content of each peptide with the label content of all peptides with which it (or immediately adjoining peptides) share any part of amino acid sequence, said comparisons being performed in a manner which allows differences in label content to be assigned to regions of amino acid sequence difference, with the preferred algorithm seeking to fit deuterium location and quantity at each location in a manner that optimizes agreement between results obtained from the plurality of fragments; and (iv) it optionally makes use of measurements of off-exchange rates of label on quenched fragments, which, by reference to the above noted site-specific rate (under quench conditions) prediction or empirical determination from endoproteinase fragmentation data of equilibrium-deuterated protein) can be employed to further sublocalize label at regions unresolved by analysis of fragments alone at one quench condition duration.

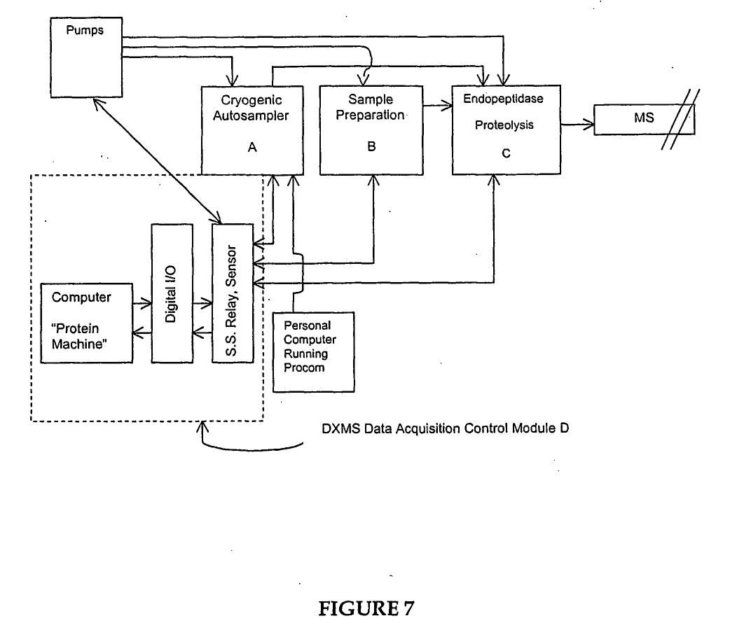

The methods of the present invention may be performed using an automated procedure. Automation may be employed to perform isotope-exchange labeling of proteins as well as subsequent proteolysis and MS-based localization procedures. The use of such automation allows one to manipulate proteolysis conditions under quench conditions, largely by employing solid-state chemistries as described above. The following discussion refers to modules as designated in the exemplary DXMS apparatus illustrated in FIG. 7.

The fluidics of the DXMS apparatus consists of a number of pumps, high pressure switching valves and electric actuators, along with connecting tubing, mixing tees, and one way flow check valves and that direct the admixture of reagents and their flow over the several small stainless steel columns containing a variety of proteins and enzymes coupled to perfusive (Poros 20) support material.

While there is a standard configuration of these various components, the pattern of the several elements can be quickly changed to suit particular experimental requirements. DXMS fluidics consists of a “cryogenic autosampler” module (A), a “functional deuteration” or sample preparation module (B) used for automated batched processing of manually prepared samples, and a “endopeptidase proteolysis” module (C). Precise temperature control is achieved by enclosing the valves, columns, and connecting plumbing of modules A, B, and C in a high thermal-capacity refrigerator kept at about 3.8° C. (the freezing point of deuterated water), and components that have no contact with pure deuterated water are immersed in melting (regular) ice.

Module A, the “cryogenic autosampler” allows a sample set (in the range of about 10-50 samples) to be prepared manually in autosampler vials, quenched, denatured, and samples frozen at −80° C., conditions under which loss of deuterium label in the prepared samples is negligible over weeks. This allows a large number of deuterated samples to be manually prepared, and then stored away for subsequent progressive proteolysis. This capability also allows samples to be manually prepared at a distant site, and then shipped frozen to the DXMS facility for later automated analysis. This module consists of a highly modified Spectraphysics AS3000® autosampler, partially under external PC control, in which the standard pre-injection sample preparation features of the autosampler are used to heat and melt a frozen sample rapidly and under precise temperature control. Under computerized control, the autosampler's mechanical arm lifts the desired sample from the −80° C. sample well, and places it in the autosampler heater/mixer/vortexer which rapidly melts the sample at 0-5° C. The liquified sample is then automatically injected onto the HPLC column.

The necessary modifications to a such a standard autosampler may include: modification of the sample basin to provide an insulated area in which dry ice can be placed, resulting in chilling of the remaining areas of the sample rack to −50 to −80° C.; placement of the autosampler within a 0-5° C. refrigerator, and “stand-off” placement of the sample preparation and sample injection syringe assemblies of the autosampler outside the refrigerator, but with otherwise nominal plumbing and electrical connection to the autosampler. An external personal computer (PC) (running Procom, and a dedicated Procrom script “Asset1”), delivers certain settings to firmware within the autosampler, allowing: (i) a much shortened subsequent post-melting dwell time of samples in the chilled basin, avoiding re-freezing of sample prior to injection; and (ii) allowing its heater/mixer to regulate desired temperatures when they are less than the default minimum temperature of 30° C.

The “sample preparation” module (B), automatically performs the “functional deuteration” or sample preparation manipulations, quench, and denaturation in large part through use of the solid-state inventions as described earlier, for example, using a protein conjugated to solid phase beads. Several components of this module will benefit from the microfluidics inventions also described earlier.

Typically, deuterated samples are manually prepared (both at 0° C., and at room temperature) by diluting 1 μL of protein stock solution with 19 μL of deuterated buffer (150 mM NaCl, 10 mM HEPES, pD 7.4), followed by “on-exchange” incubation for varying times (10 sec, 30 sec, 100 sec, 300 sec, 1000 sec, 3000 sec) prior to quenching in 30 μL of 0.5% formic acid, 2 M GuHCl, 0° C. These functionally deuterated samples are then subjected to DXMS processing, along with control samples of undeuterated and fully deuterated protein (incubated in 0.5% formic acid in 95% D₂O for 24 hours at room temperature). The centroids of probe peptide isotopic envelopes are then measured using appropriate software. In order to obtain the deuteration levels of each peptide corrected to the values after “on-exchange” incubation, but before DXMS analysis, the corrections for back-exchange are made employing the methods of Zhang and Smith as previously described.

Regardless of the manner of sample preparation, quenched samples are then automatically directed to the “endopeptidase proteolysis” module (C), in which proteolysis is accomplished using a battery of solid-state protease columns, variously pepsin, fungal protease XIII, newlase, etc. as desired, with the resulting peptide fragments being collected on a small reversed-phase HPLC column, with or without the use of a small c18 collecting pre-column. This column(s) is then acetonitrile gradient-eluted, with optional additional post-LC on-line proteolysis. The effluent is then directed to the electrospray head of the mass spectrometer (a Finnegan ion trap or a Micromass Q-TOP) which protrudes into a hole drilled in the side of the refrigerator. Several components of this module lend themselves to microfluidic devices as described earlier.

In a preferred embodiment, the endopeptidase proteolysis module consists of four high pressure valves (Rheodyne 7010); with valve 1 bearing a 100 μL sample loop; valve 2 bearing a column (66 μl bed volume) packed with porcine pepsin coupled to perfusive HPLC support material (Upchurch Scientific 2 mm×2 cm analytical guard column; catalog no. C.130B; porcine pepsin, Sigma catalog no. p6887, coupled to Poros 20 AL media at 40 mg/mL, in 50 mM sodium citrate, pH 4.5, and packed at 9 mL/min according to manufacturer's instructions); valve 3 bearing a C18 microbore (1 mm×5 cm) reversed phase HPLC column (Vydac catalog no. 218MS5105), and valve 4 connected to the electrospray head of a mass spectrometer. Inline filters (0.05 um, Upchurch catalog no. A.430) are placed on each side of the pepsin column, and just before the C18 column (Vydac prefilter, catalog no. CPF 10) to minimize column fouling and carryover from aggregated material.

In this configuration, four HPLC pumps (Shimadzu LC-10AD, operated by a Shimadzu SCL-10A pump controller) supplied solvents to the valves; with pumps C and D providing 0.05% aqueous TFA to valve 1 and valve 2 respectively; pumps A (0.05% aqueous TFA) and B (80% acetonitrile, 20% water, 0.01% TFA) are connected through a microvolume mixing tee (Upchurch catalog no. P.775) to provide valve 3 with the C18 column-eluting gradient. All valves are connected to Two-Position Electrical Actuators (Tar Designs Inc.).

A typical sample is processed as follows: a 20 μL of hydrogen exchanged protein solution is quenched by shifting to pH 2.2-2.5, 0° C. with a 30 μL of quenching stock solution chilled on ice. The quenched solution is immediately pulled into the sample loading loop of valve 1, and then the computer program (see below) started. Pump C flow (0.05% TFA at 200 μL/min) pushes the sample out of injection loop onto the C18 HPLC column via the solid-state pepsin column at valve 2 (digestion duration of about 26 seconds). After two minutes of pump C solvent flow, the C18 column is gradient-eluted by pumps A and B linear gradient from 10 to 50% B over 10 minutes; 50 μL/min; pumps A, 0.05% TFA; pump B, 80% acetonitrile, 20% water, 0.01% TFA), with effluent directed to the mass spectrometer. During data acquisition, pump D (aqueous 0.05% TFA 1 mL/min, 10 minutes) back-flushes the pepsin column to remove retained digestion products.

The timing and sequence of operation of the foregoing DXMS fluidics may be controlled by a personal computer running a highly flexible program in which sequential commands to targeted solid state relays can be specified, as well as variably timed delays between commands, as illustrated in the “DXMS Data Acquisition Control Module” (D). Certain command lines may access an array matrix of on- and off-exchange times, and the entire sequence of commands may be set to recycle, accessing a different element of the array with each cycle executed. Certain command lines may be set to receive “go” input signals from peripherals, to allow for peripheral-control of cycle progression. A library of command sequences may be prepared, as well as a library of on/off time arrays. An exemplary protein machine program can be configured to execute a supersequence of command sequence-array pairs.

An exemplary protein machine program (written in LabView I, National Instruments, Inc) controls the state(s) of a panel of solid-state relays on backplanes (SC-206X series of optically isolated and electromechanical relay boards, National Instruments, Inc.) with interface provided by digital input/output boards (model no. PCI-DIO-96 and PCI-6503, with NI-DAQ software, all from National Instruments, Inc.). The solid-state relays in turn exert control (contact closure or TTL) over pumps, valve actuators, and mass spectrometer data acquisition. Each of these peripherals is in turn locally programmed to perform appropriate autonomous operations when triggered, and then to return to their initial conditions. The autosampler and HPLC column pump controller are independently configured to deliver a “proceed through delay” command to the Digital I/O board as to insure synchronization between their subroutines and the overall command sequence.

In order to optimize or “tune” endopeptidase proteolysis, preliminary proteolytic “tuning” studies are performed to establish the fragmentation conditions (compatible with slowed exchange) optimal for peptide generation from the target polypeptide. Two major parameters that are often optimized are the concentration of GuHCl in quenching buffer and the pump C flow rate over the pepsin column. Typically, a 1 ml stock solution of protein (10 mg/ml, pH 7.0) is diluted with 19 mL of water and then quenched with 30 mL of 0.5% formic add containing various concentrations of GuHCl (0-6.4 M). The quenched sample is then pulled into the sample loading loop, and the DXMS program sequence triggered immediately after sample loading. The flow over the pepsin column is varied (100 μL/min-300 μL/min) to adjust the duration of proteolytic digestion.

In order to quickly identify pepsin generated peptides for each digestion condition employed, spectral data is preferably acquired in particular modes, for example designated herein as “triple play” and “standard double play” modes, which have been empirically tuned to optimize the number of different parent ions upon which MS2 is performed. This data is then analyzed by appropriate software.

Triple play consists of three sequentially executed scan events; first scan, MS1 across 200-2000 m/z; second scan, selective high resolution “zoom scan” on most prevalent peptide ion in preceding MS1 scan, with dynamic exclusion of parents previously selected; and third scan, MS2 on the same parent ion as the preceding zoom scan. The triple play data set or double play data set is then analyzed employing the Sequest software program (Finnigan Inc.) set to interrogate a library consisting solely of the amino acid sequence of the protein of interest to identify the sequence of the dynamically selected parent peptide ions.

This tentative peptide identification is verified by visual confirmation of the parent ion charge state presumed by the Sequest program for each peptide sequence assignment it made. This set of peptides is then further examined to determine if the “quality” of the measured isotopic envelope of peptides was sufficient (adequate ion statistics, absence of peptides with overlapping m/z) to allow accurate measurement of the geometric centroid of isotopic envelopes on deuterated samples.

According to an additional aspect of the present invention, it may be useful to perform in vivo analysis of a polypeptide of interest, for example, in situ analysis of protein-binding partner interactions. In such applications, the protein, while present in its native environment as a component of an intact living cell, or as a component of a cellular secretion such as blood plasma, is on-exchanged by incubating cells or plasma in physiologic buffers supplemented with tritiated or deuterated water. The binding partner is then added, allowed to complex to the cell or plasma-associated protein, and then off-exchange initiated by returning the cell or plasma to physiologic conditions free of tritiated or deuterated water. During the off-exchange period (hours to days) the formed protein-binding partner complex is isolated from the cell or plasma by any purification procedure which allows the complex to remain continuously intact. At the end of the appropriate off-exchange period, fragmentation and analysis of purified complex proceeds as above. This analytic method is especially appropriate for proteins that lose substantial activity as a result of purification, as the binding site is labeled prior to purification.

According to another aspect of the present invention, binding site analysis may be performed using indirect hydrogen exchange. In the methods described above, the entire surface of the protein is labeled initially, and label is then removed from those surfaces which remain solvent exposed after formation of the complex of the binding protein and its binding partner. The binding site of the protein is occluded by the binding partner, and label is therefore retained at this site.

When the complex is formed, the binding protein may undergo changes in conformation (allosteric changes) at other sites, too. If these changes result in segments of the protein being buried which, previously, were on the surface, those segments will likewise retain label.

It is possible to distinguish binding site residues from residues protected from “off-exchange” by allosteric effects. In essence, the binding partner, rather than the binding protein, is labeled initially. The binding protein is labeled indirectly as a result of transfer of label from the binding partner to the binding protein. Such transfer will occur principally at the binding surface.

This procedure will functionally label receptor protein amides if they are slowed by complex formation and are also in intimate contact with the binding partner in the complexed state. Receptor protein amides that are slowed because of complex formation-induced allosteric changes in regions of the protein, which are not near the protein-binding partner interaction surface will not be labeled. This procedure may be performed as follows. First, binding partner is added to labeled water (preferably of high specific activity) to initiate exchange labeling of the binding partner.

After sufficient labeling is achieved, binding partner is then separated from the excess of solvent isotope under conditions that produce minimal loss of label from the binding partner. This can be accomplished, for example, by shifting the buffer conditions to those of slowed exchange (0° C., acidic pH) followed by G-25 spin column separation of the binding partner into isotope-free buffer, or by employing stopped-flow techniques in which the on-exchange mixture is rapidly diluted with large volumes of isotope free buffer.

The labeled binding partner, now essentially free of excess solvent isotope, is added to receptor protein and conditions adjusted to allow spontaneous reversible (equilibrium) complex formation to take place between the two. The conditions of temperature and pH should also allow, and preferably maximize, the specific transfer of label from the labeled binding partner to amides on the binding protein's interaction surface with partner. Typically, the pH will be in the range of about 5-8 (conducive to ligand binding) and a temperature in the range of about 0-37° C. Initially, use of pH 7 and 22° C. is preferable, with the transfer being controlled by controlling the incubation time. A typical trial incubation time would be 24 hours. These conditions of pH, temperature and incubation time may of course be varied.

The complex is then incubated for periods of time sufficient to allow transfer of label from the labeled binding partner to the receptor protein. During this incubation period, label which has on-exchanged to regions of the binding partner that are distant from the receptor-binding partner interaction surface will leave the binding partner by exchange with solvent hydrogen and be rapidly and highly diluted in the large volume of solvent water, thereby preventing its efficient subsequent interaction with the binding protein. However, label that has been attached to binding partner amides present within the (newly formed) protein-binding partner interaction surface will be capable of exchanging off of the binding partner only during the brief intervals when the interaction surface is exposed to solvent water, i.e., when the complex is temporarily dissociated. When so dissociated and solvent exposed, a portion of tritium present on amides within the binding partner's interaction surface will leave the surface and for a brief time, remain within the proximity of the surface. Given the rapid (essentially diffusion limited) rebinding of binding protein and partner, much of the released tritium that (briefly) remains within the environs of the partner's binding surface will in part exchange with amides on the (future) interaction surface of the approaching binding protein molecule that subsequently binds to the binding partner. Once such binding occurs, the transferred label is again protected from exchange with solvent until the complex dissociates again. The result will be the progressive transfer of a portion of the label from the binding partner interaction surface to exchangeable amides on the cognate protein interaction surface.

Amides whose exchange rates are conformationally slowed each time complex formation occurs can also become labeled, but they will do so at a much slower rate than amides within the binding surface, as they are located more distant from the high concentration of label “released” at the interaction surface with each complex dissociation event. The efficiency of transfer is roughly inversely proportional to the cube of the distance between such conformational changes and the binding surface.

The binding protein-labeled binding partner complex incubation conditions are adjusted to optimize specific interaction surface amide tritium transfer (SISATT) for a articular binding protein-partner pair. SISATT is defined as the ratio of the amount of tritium (CPM) transferred from binding partner to binding protein peptide amides previously determined to undergo slowing of amide hydrogen exchange upon binding-protein partner complex formation divided by the total tritium (CPM) transferred from binding partner to all peptide amides in the binding protein.

After an incubation period that allows and preferably maximizes SISATT, the conditions of slow hydrogen exchange are restored, the complex is dissociated and the binding protein fragmented. Fragments of binding protein (as opposed to the initially labeled binding partner) that bear label are identified, and further characterized as previously described. Preferably, deuterium is used instead of tritium as the label. Deuterium has the advantage of allowing a much higher loading of label (since deuterium is much cheaper than tritium).

It is possible, also, to directly label the binding partner with deuterium and the binding protein with tritium. As a result, both the binding site and allosterically buried amides of the binding protein will be tritiated, but only binding site amides will be deuterated.

The indirect method is especially applicable to study of proteins that undergo substantial conformation of changes after, or in the course of binding, such as insulin and its receptor.

According to further aspects of the present invention, after determining the binding sites of a binding protein or a binding partner, by the present methods (alone or in conjunction with other methods), the information may be exploited in the design of new diagnostic or therapeutic agents. Such agents may be fragments corresponding essentially to said binding sites (with suitable linkers to hold them in the proper spatial relationship if the binding site is discontinuous), or to peptidyl or non-peptidyl analogues thereof with the similar or improved binding properties. Alternatively, they may be molecules designed to bind to said binding sites, which may, if desired, correspond to the paratope of the binding partner.

The diagnostic agents may further comprise a suitable label or support. The therapeutic agents may further comprise a carrier that enhances delivery or other improves the therapeutic effect

The agents may present one or more epitopes, which may be the same or different, and which may correspond to epitopes of the same or different binding proteins or binding partners.

Alternative embodiments of the present invention are apparent to one of skill in the art. The following embodiments are intended to provide additional useful applications of the DXMS method of the present invention.

A method of characterizing the binding site of a binding protein, said method comprising:
labeling said protein with an isotope of hydrogen other than ¹H in the presence and absence of a binding partner for said protein, and comparing the pattern of labeling obtained on said protein in the presence and absence of said binding partner,
wherein the location of label is determined by generating a population of endopeptidase fragments of said labeled protein under conditions of slowed hydrogen exchange and then deconvoluting fragmentation data acquired from said endopeptidase fragments.

A method of characterizing the binding site of a binding protein, said method comprising:
labeling said protein with an isotope of hydrogen other than ¹H;
contacting said labeled protein with a binding partner for said protein; and
off-exchanging the resulting binding pair;
wherein the location of label is determined by generating a population of endopeptidase fragments of said labeled protein under conditions of slowed hydrogen exchange and then deconvoluting fragmentation data acquired from said endopeptidase fragments.

A method of determining peptide amides that are near residues important in the interaction between a binding protein and a high affinity binding partner therefor, said method comprising:
labeling said protein with an isotope of hydrogen other than ¹H in the presence and absence of a binding partner for said protein, and comparing the pattern of labeling obtained on said protein in the presence and absence of said binding partner,
wherein the location of label is determined by generating a population of endopeptidase fragments of said labeled protein under conditions of slowed hydrogen exchange and then deconvoluting fragmentation data acquired from said endopeptidase fragments.

A method of determining peptide amides that are near residues important in the interaction between a binding protein and a high affinity binding partner therefor, said method comprising:
labeling said protein with an isotope of hydrogen other than ¹H;
contacting said labeled protein with a binding partner for said protein; and
off-exchanging the resulting binding pair;
wherein the location of label is determined by generating a population of endopeptidase fragments of said labeled protein under conditions of slowed hydrogen exchange and then deconvoluting fragmentation data acquired from said endopeptidase fragments.

A method of screening test compounds to determine whether any compounds mimic the interaction of a binding protein and a high affinity binding partner therefor, said method comprising:
determining which peptide amides of said protein are near residues important in said interaction according to the method of claim 34; and
determining the location of isotope(s) on the labeled peptides produced by on-exchanging said protein with said isotope of hydrogen and off-exchanging the resulting labeled protein in the presence and absence of said test compound;
wherein a similar location of isotope(s) on the labeled peptides identified in the presence of said test compound, as compared to the location of isotope(s) on the labeled peptides identified in the presence of said high affinity binding partner, is indicative of a test compound that mimics the interaction of a binding protein and a high affinity binding partner therefor.

A method of screening test compounds to determine whether any compounds mimic the interaction of a binding protein and a high affinity binding partner therefor, said method comprising:
determining which peptide amides of said protein are near residues important in said interaction according to the method of claim 35; and
determining the location of isotope(s) on the labeled peptides produced by on-exchanging said protein with said isotope of hydrogen, contacting said protein with said test compound, and off-exchanging said labeled protein;
wherein a similar location of isotope(s) on the labeled peptides identified in the presence of said test compound, as compared to the location of isotope(s) on the labeled peptides identified in the presence of said high affinity binding partner, is indicative of a test compound that mimics the interaction of a binding protein and a high affinity binding partner therefor.

A method of determining the surface conformation of a polypeptide, said method comprising:
labeling said protein with an isotope of hydrogen other than ¹H,
determining the quantity of isotope and rate of exchange of hydrogen at a peptide amide hydrogen with said isotope by generating a population of sequence-overlapping endopeptidase fragments of said labeled protein under conditions of slowed hydrogen exchange and then deconvoluting said fragmentation data acquired from said endopeptidase fragments, and
comparing the rates of exchange of a plurality of peptide amide hydrogens to determine the surface accessibility of peptide amide hydrogens.

A method of determining a conformational change in a polypeptide, said method comprising:
independently labeling more than one conformer of said protein with an isotope of hydrogen other than ¹H
determining the quantity of isotope and/or rate of exchange of hydrogen at a peptide amide hydrogen with said isotope in said conformers by generating a population of sequence-overlapping endopeptidase fragments of said labeled conformers under conditions of slowed hydrogen exchange and then deconvoluting fragmentation data acquired from said endopeptidase fragments, and
comparing the fragmentation data acquired from said conformers wherein a change in said data reflective of a difference in labeling of said peptide amide hydrogen(s) is indicative of a conformational change.

A method of determining a conformational change in a polypeptide as a result of a change in conditions within which the polypeptide is present, said method comprising:
labeling said protein with an isotope of hydrogen other than ¹H at two or more conditions,
determining the quantity of isotope and/or rate of exchange of hydrogen at a peptide amide hydrogen with said isotope by generating a population of endopeptidase fragments of said labeled protein under conditions of slowed hydrogen exchange and then deconvoluting fragmentation data acquired from said endopeptidase fragments, and
comparing the fragmentation data acquired at each condition, wherein a change in said data reflective of a difference in labeling of said peptide amide hydrogen(s) is indicative of a conformational change in said polypeptide as a result of a change in conditions.

A method of determining a conformational change in a polypeptide as a result of a change in conditions within which the polypeptide is present, said method comprising:
labeling said protein with an isotope of hydrogen other than ¹H;
off-exchanging said labeled protein following a change in conditions;
determining the quantity of isotope and/or rate of exchange of hydrogen at a peptide amide hydrogen with said isotope by generating a population of endopeptidase fragments of said labeled protein under conditions of slowed hydrogen exchange and then deconvoluting fragmentation data acquired from said endopeptidase fragments; and
comparing the fragmentation data acquired, wherein a change in said data reflective of a difference in labeling of said peptide amide hydrogen(s) is indicative of a conformational change in said polypeptide as a result of a change in conditions.

A method of determining, at a resolution of about 1-5 amino acid residues, the position of peptide amine group(s) that have been hydrogen-exchanged labeled with deuterium in a protein of known amino acid sequence, said method comprising:
(a) placing the labeled protein under conditions of slowed hydrogen exchange;
(b) fragmenting the labeled protein with endopeptidase(s) to produce a population of sequence-overlapping fragments, wherein more than half of said fragments differ by 1-5 amino add residues;
(c) quantifying the amount of deuterium label on a plurality of members of said population by mass spectrometry; and
(d) comparing the amount of deuterium label on at least one member of said population with the amount of deuterium label on at least one other member of said population;
   thereby localizing the position of the deuterium-labeled peptide amide group in the protein to within about 1-5 amino acid residues.

The invention will now be described in greater detail by reference to the following non-limiting examples.

### EXAMPLE 1

### Protein Expression and Purification

The sequence of mouse D-AKAP2 (Genbank Accession No. AF021833) was sub-cloned into pET-15b (Invitrogen), using NdeI and XhoI restriction sites after mutating an internal NdeI site. As a result of cloning, three non-native amino acids were attached to the N terminus of the protein after thrombin cleavage. The plasmid was transformed into BL21 (DE3) cells (Novagen) and grown in LB medium with 100 μg/ml of ampicillin at 37° C., 300 rpm. The cells were induced at 0.8 (A_{600 nm}) with 0.5 mM IPTG and the protein expressed for five hours at 24° C. Six liters of culture were pelleted and lysed in 100 ml of lysis buffer (20 mM Tris (pH 8.0), 150 mM NaCl, 5 mM benzamidine) using a French press (1000 psi:1 psi ≈6.9 kPa). The lysate was centrifuged at 17,000 g for 30 minutes at 4° C. The protein was purified from the supernatant using Talon resin (Clontech) and the histidine (His) tag cleaved with three milligrams of thrombin (Sigma). The protein was dialyzed into gel-filtration buffer (20 mM MOPS (pH 7.0), 200 mM NaCl, 2 mM EDTA, 2 mM EGTA, 1 mM DTT) and further purified using an S200 gel-filtration column (Pharmacia). The protein concentration was determined using the absorbance at 280 nm and an extinction coefficient of 0.99 ml mg⁻¹cm⁻¹, calculated by the method of Pace et al. (Protein Sci. 4:2411-2423, 1995).

Murine RIIa was expressed in BL21 (DE3) cells and lysed in lysis buffer (20 mM MES (pH 6.5), 100 mM NaCl, 2 mM EDTA, 2 mM EGTA, 2 mM DTT, 5 mM Benzamidine) as described above. RIIa was precipitated from the supernatant by adding 45% (w/v) ammonium sulfate and incubated for one hour. The precipitated protein was solublized in lysis buffer and added to a cAMP affinity resin (Sigma). After binding to the resin overnight at 4° C., the protein was washed with lysis buffer containing 1M NaCl followed by lysis buffer. RIIa was eluted for 30 minutes at room temperature using 25 mM cGMP (Sigma) in lysis buffer (pH 5.1). The protein was dialyzed overnight at 4° C. in gel-filtration buffer (50 mM MES (pH 5.8), 200 mM NaCl, 2 mM EDTA, 2 mM EGTA, 2 mM DTT) and further purified using an S200 gel-filtration column (Pharmacia). The protein concentration was determined using an extinction coefficient of 0.69 ml mg 1 cm-1, on the basis of amino acid analysis.

### EXAMPLE 2

### Circular Dichroism

The spectrum was obtained using an AVIV 202 spectropolarimeter in a 0.1 cm rectangular, quartz cuvette (AVIV). The time constant for data collection was 100 ms with a 4 seconds averaging time. Three acquisitions were averaged. The buffer (20 mM NaPO₄, 150 mM NaCl, pH 7.0) spectrum was subtracted from the sample spectrum The protein concentration was 2 μM. The mean residue ellipticity (MRE) was calculated according to the following equation:
MRE=θobs(M/10lcn)
where ζobs is the buffer corrected ellipticity, M is the molecular weight (42,800 Da) of D-AKAP2, 1 is the pathlength, c is the concentration (in molarity) and n is the number of amino acid residues in the protein.

### EXAMPLE 3

### Limited Proteolysis and Boundary Mapping

D-AKAP2 (0.85 mg/ml) was digested separately with both trypsin (Worthington Biochemical Corporation) and endoproteinase Glu-C (Boehringer Mannheim) in a 1:100 (w/w) ratio. At various time-points, aliquots were taken and quenched with 10% (v/v) glacial acetic acid for the trypsin digests or quick-frozen on solid CO₂ for the endoproteinase digests. The samples were analyzed by SDS-PAGE and by liquid chromatography-mass spectrometry (LCMS). For each of the various digest time-points, 5.0 μg of total digested protein was loaded onto a Michrom BioResources Magic 2002 microbore HPLC system (Auburn, Calif.) equipped with a 1.0 mm×150 mm Vydac C4 column (5 μm, 300 Å), equilibrated at a flow rate of 50 μl/min and a column temperature of 35° C. A gradient from 10 to 80% solvent B over 60 minutes was then initiated (solvent A, 2% (v/v) acetonitrile, 0.1% (v/v) trifluoroacetic acid (TFA); solvent B, 90% acetonitrile, 0.095% TFA). The eluent from the UV detector of the HPLC was plumbed without splitting into a PE Sciex/Applied Biosystems QSTAR quadruple-TOF mass spectrometer (Foster City, Calif.). Spectra from 400 to 2000 m/z were recorded continuously over the entire gradient with the CAD gas set to 3 and the following source parameters: ionspray gas=60, ionspray voltage=5 kV, and curtain gas=20.

### EXAMPLE 4

### Surface Plasmon Resonance

Binding of RIIa to D-AKAP2 was performed using a BIAcore 3000 (BIAcore). The regulatory subunit of PKA was immobilized to a CM5 chip (BIAcore) as described (Herberg et al., J. Mol. Biol., 298:329-339, 2000). Using this method, 80 response units (RU) of a cAMP analog (8-(6-aminohexyl)aminoadenosine-3′,5′-cyclic monophosphate (8-AHA cAMP) (BioLog) were covalently immobilized to flow channel 1 (FC1) and flow channel 2 (FC2) of an activated CM5 chip (BIAcore) using the amine coupling kit from BIAcore. RIIa (cAMP free) was injected over FC2 at a flow rate of 50 μl/minute for 1 minute, resulting in 450 RU of bound RIIa. Serial dilutions (250-2 nM) of D-AKAP2 were injected over FC1 and FC2 at 50 μl/minute for 2 minutes with a 4 minute dissociation time using the kinject injection mode. The surface was regenerated by injecting 0.2% (w/v) SDS and running buffer (10 mM HEPES, 150 mM NaCl, 3 mM EDTA, 0.005% (w/v) P20 (HBS-EP, BIAcore) for 30 seconds at 50 μl/min. Three replicates were obtained using the automated Kinetic Analysis module provided in the BIAcore instrument software. Each replicate was fit separately to a 1:1 binding model using BIAevaluation software V. 3.0 (BIAcore). The rates and dissociation constants from each fit were averaged.

### EXAMPLE 5

### Deuterium Exchange-Mass Spectrometry (DXMS) Analysis

A. General Operation Procedure.

A 20 μl hydrogen-exchanged protein solution was quenched by shifting to pH 2.2-2.5, 0° C. with 30 μl of 0.8% formic acid with various concentrations of GuHCl (the final pH was measured on a non-deuterated mock solution at room temperature using a pH meter (model 250, Denver Instrument Co., Arvada, Colo.)). At 0° C., the quenched solution was immediately passed over a column (66 μl bed volume; Upchurch Scientific, catalog number C.130B) filled with porcine pepsin (Sigma, catalog number p6887) immobilized on Poros 20 AL media at 30 mg/ml following the manufacturer's instructions, with 0.05% TFA (200 ul/min) for two minutes with contemporaneous collection of proteolytic products by a C18 column (Vydac, catalog number 218MS5105). Inline filters (Upchurch catalog number A.430) were placed on each side of the pepsin column, and just before the C18 column (Vydac prefilter, catalog number CPF 10) to minimize column fouling. Subsequently the C18 column was eluted with a linear gradient of 10-50% solvent B over 10 minutes (solvent A was 0.05% TFA in water, and solvent B was 80% acetonitrile, 20% water, 0.01% TFA). Mass spectrometric analyses were carried out with a Finnigan LCQ electrospray mass spectrometer with capillary temperature at 200° C.

B. Sequence Identification of Pepsin-Generated Peptides.

To quickly identify pepsin-generated peptides for each digestion condition employed, spectral data was acquired in “triple play” mode. The triple play data set was then analyzed employing the Sequest software program (Finnigan, Inc.) to identify the sequence of the dynamically selected parent peptide ions. This tentative peptide identification was verified by visual confirmation of the parent ion charge state presumed by the Sequest program for each peptide.

C. Deuterium Exchange Experiments.

Deuterated samples were prepared (both at 0° C. and at 22° C.) by diluting 1 μl of D-AKAP2 stock solution with 19 μl of deuterated buffer (10 mM HEPES (pD 7.4), 150 mM NaCl), followed by “on-exchange” incubation for varying times (10-3000 seconds) prior to quenching in 30 μl of 0.5% formic acid, 2 M GuHCl, 0° C. These functionally deuterated samples were then subjected to DXMS processing as above, along with control samples of non-deuterated and fully deuterated D-AKAP2 (incubated in 0.5% formic acid in 95% 2H₂O for 24 hours at 22° C.). The centroids of probe peptide isotopic envelopes were measured using the Magtran program provided by Zhongqi Zhang. The corrections for back-exchange were made employing the methods of Zhang and Smith (Protein Sci. 2:522-531, 1993),

$$Deuteration Level ( \% ) = \frac{m ( P ) - m ( N )}{m ( F ) - m ( N )} \times 100$$

$$Deuterium Incorporation ( no . ) = \frac{m ( P ) - m ( N )}{m ( F ) - m ( N )} \times max D$$

where n(P), m(N), and m(F) are the centroid value of partially deuterated peptide, non-deuterated peptide, and fully deuterated peptide, respectively; maxD is the maximum deuterium incorporation calculated by subtracting the number of proline residues in the third or later amino acid residue and two from the number of amino acid residues in the peptide of interest (assuming the first two amino acids can not retain deuterons (Bai et al., Proteins: Struct. Funct. Genet. 17:75-86, 1993)). Typical deuteron recovery of fully deuterated sample ((m(F)−in(N))/maxD) was 80-90%.

D. Computational Analysis and Three-dimensional Structure Homology Modeling

Sequence alignments were done using ClustalX, a Windows-ported version of ClustalW (Thompson et al., Nucl. Acids Res. 22:4673-4680, 1994). Domain searches against the Pfam domain database (Bateman et al., Nucl. Acids Res. 27:260-262, 1999) were performed using HMMER 2.1.1 (Durbin et al., Biological Sequence Analysis: Probabilistic Models of Proteins and Nucleic Acids, Cambridge University Press, 1998). A three-dimensional model of the RGS domain B of mouse D-AKAP2 was created using the SwissModel server (Peitsch, Biochem. Soc. Trans. 24:274-279, 1996). A suitable template was identified by performing a BLAST search (Altschul et al., J. Mol. Biol. 215:403-410, 1990) of PDB using the sequence of the RGS B domain of D-AKAP2 as query. The coordinates of chain E from the X-ray crystal structure of the complex Of Alf4-Activated Gi-Alpha-1 with RGS4 from rat (PDB ID=1AGR_E) were used as template. Initial structural alignments were created using the first approach mode of the SwissModel server and the final model of the three-dimensional structure was calculated on the basis of this preliminary model, utilizing the optimize mode of the software.

The model was subjected to additional energy minimization using the GROMOS 43B1 force-field as implemented in the DeepBlue SwissPDB Viewer (Guex and Peitsch, Electrophoresis 18:2714-2723, 1997) using 5000 cycles of steepest descent followed by 5000 cycles of conjugate gradients. All computations were done in vacuo, without reaction field. Harmonic constraints of 50 C-factors and 2500 C-factors were used for each method.

The final model was validated using the Biotech Validation Suite for Protein Structures (available on the world wide web at the URL “biotech.ebi.ac.uk:8400”), which includes the packages PROCHECK V3.5 (Laskowski et al., J. Appl. Crystallog. 26:283-291, 1993) PROVE V2.3 (Pontius et al., J. Mol. Biol. 264:121-136, 1996), and WHAT IF V4.99 (Rodriguez et al., Bioinformatics 14:523-528, 1998). Structure models were visualized and images of structures were rendered with WebLab Pro v4.0 (Accelrys). Structural sequence alignments were performed using the CE (Combinatorial Extension) method (Shindyalov and Bourne, Protein Eng. 11:739-747, 1998).

### EXAMPLE 6

### DXMS Analysis used to Elucidate the Domain Organization of D-AKAP2

A. Background.

For a cell to communicate with its environment, a dynamic network of protein signaling molecules must transmit an external signal to the interior of the cell. The spatial and temporal organization of these protein assemblies is essential to ensure specificity in cell signaling. Over the past decade A-kinase anchoring proteins (AKAPs) have been recognized as integral in coordinating the specificity of signaling through cAMP-dependent protein kinase (PKA) by localizing the kinase near its substrate targets (Colledge and Scott, Trends Cell. Biol. 9:216-221, 1999; Edwards and Scott, Curr. Opin. Cell. Biol. 12:217-221, 2000). AKAPs are multidomain proteins containing a PKA kinase binding (AKB) domain, which interacts with the regulatory subunit of PKA, and a targeting domain, which directs the AKAP to various sub-cellular locations. In addition to anchoring PKA, AKAPs have also been shown to bind other signal transduction proteins thus providing a scaffold for coordination of signaling complexes (Dodge and Scott, FEBS Lett. 476:58-61, 2000). Despite the important role that AKAPs play in localizing PKA activity, little is known about their overall domain organization and their ability to regulate signaling complexes.

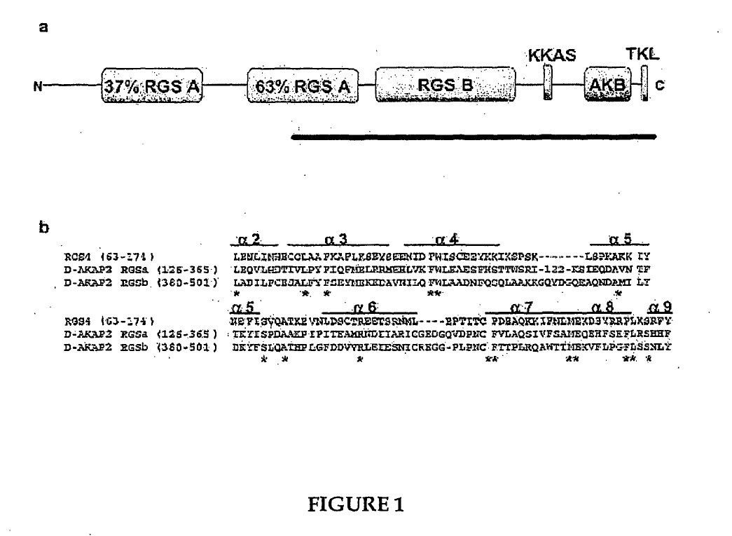

Dual-specific A-kinase anchoring protein 2 (D-AKAP2), initially identified from a genomic screen, binds to both type I and type II regulatory subunit isoforms of PKA (Huang et al., Proc. Natl. Acad. Sci. USA 94:11184-11189, 1997). This protein is expressed in nearly all tissues and localizes to the mitochondria (Wang et al., Proc. Natl. Acad. Sci. USA 98:3220-3225, 2001). The specific PKA signaling pathway that D-AKAP2 regulates is unknown; however, interaction partners are suggested by sequence homology (see FIG. 1A). There are two putative regulators of G-protein signaling (RGS) domains, distinct from the PKA interaction site that could coordinate upstream G-protein signaling events with downstream PKA signaling (Wang et al., supra). There is also a putative PDZ (PSD95/Dlg/ZO1) binding motif at the C terminus that could serve as a motif for targeting to membrane receptor complexes (Sheng and Sala, Ann. Rev. Neurosci. 24:1-29, 2001). To verify the multi-domain organization of D-AKAP2, limited proteolysis and enhanced methods of deuterium exchange-mass spectrometry (DXMS) were used to determine the folded regions of this multi-domain protein in solution and to evaluate the role of the domain organization on PKA binding.

Amide hydrogen exchange has proven to be an increasingly powerful method by which protein dynamics, structure and function can be studied (Englander et al., Prot. Sci. 6:1101-1109, 1997; and Engen and Smith, Anal. Chem. 73:256A-265A, 2001). These studies can provide information that greatly amplifies and refines inferences drawn from high-resolution structural studies, and can provide unique insights when reliable structural information is unavailable. Deuterium exchange methodologies coupled with liquid chromatography mass spectrometry (LCMS), developed over the past ten years, presently provide the most effective approach to study proteins larger than 30 kDa. Proteolytic and/or collision-induced dissociation (CID) fragmentation methods allow exchange behavior to be mapped to subregions of the protein. Amide hydrogen exchange studies are expected to play a central role in deciphering proteomic structure, function and dynamics over the next decade.

The methods of the present invention used herein to study D-AKAP2 use DXMS to obtain 99% sequence coverage of mouse D-AKAP2 by exchange-assessable peptide fragments, allowing the identification and resolution of this protein's domain organization in solution. A structural model of the RGS domain of D-AKAP2 is provided and it is shown that regions of higher deuteron incorporation were located in loops and turns, whereas regions of lower incorporation were located onto α-helices. Thus, DXMS is used in the present invention to validate the structural model of the RGS domain and provides valuable insights into the domain organization of D-AKAP2 and its role on PKA binding.

B. Results of DXMS Analysis of D-AKAP2.

i. Computational Domain Analysis.

Human D-AKAP2 was used as the query sequence to perform a RPS-BLAST (Altschul et al., Nucl. Acids Res. 25:3389-3402, 1997) search of the NCBI CDD database (version 1.54). Two domains that matched Smart 000315 and Pfam PF00615 models were identified as RGS domains (see FIG. 1A). The domain closer to the N terminus (RGS A) was split in two parts, corresponding to 37% and 63% of the model length. A 124 amino acid residue insert separated the two regions. The first region of RGS A (Smart E-value=1×10⁻⁴) encompassed residues 125-168, whereas the second region (Smart E-value=7×10⁻¹²) included residues 292-368. The second RGS domain (RGS B) included residues 380-505 (Smart E-value=1×10⁻¹⁸). E-values and boundaries reported by Pfam were consistent with those obtained from Smart. To determine the structural location of the RGS A insert, the region including the first RGS domain of D-AKAP2 was aligned with the sequence corresponding to the crystal structure of rat RGS4 (PDB 1AGR, chain 2) (Tesmer et al., Cell 89:251-261, 1997). On the basis of the alignment, the insertion was predicted to lie between α-helices 4 and 5 of the RGS domain (see FIG. 1B). The hydrophobic residues that comprise the core of the RGS domain appeared to be conserved for both D-AKAP2 RGS domains.

ii. Protein Expression.

Although attempts to purify the full-length, human protein from a bacterial expression system were unsuccessful, the initial mouse D-AKAP2 described in Huang et al. (supra), which corresponded to residues 291-662 of the human clone, was expressed in a soluble form and used as a model system (see FIG. 1A).

iii. The Folded State of Truncated Mouse D-AKAP2.

To evaluate the folded state of truncated, soluble D-AKAP2 (375 amino acid residues), the protein was characterized using circular dichroism (CD) spectroscopy and limited proteolysis. The CD spectrum of D-AKAP2 contained two local minima, at 209 nm and 223 nm, indicative of a mostly α-helical protein. In addition to forming stable secondary structure, limited proteolysis revealed two protease-resistant domains that were inaccessible to both trypsin and Glu-C proteases. Domain boundaries were mapped using mass spectrometry after one hour and after 24 hours of digestion (see Table 1). After 24 hours of digestion, both trypsin and Glu-C further cleaved the N terminus of the larger domain leaving a stable core containing the putative RGS domain (residues 93-214). The smaller domain contained the AKB domain (residues 336-360) and was not detected after 24 hours of digestion with either protease.

TABLE 1
Peptide fragments from limited proteolysis identified
by mass spectrometry.

| | Large domain | | Small domain | |
|---|---|---|---|---|
| Proteolysis time (h) | Trypsin | Glu-C | Trypsin | Glu-c |
| 1 | 35-265 | 32-276 | 270-371 | 277-375 |
| 24 | 75-265 | 85-276 | | 314-375 |

iv. Binding of D-AKAP2 to the Regulatory Subunit of PKA.

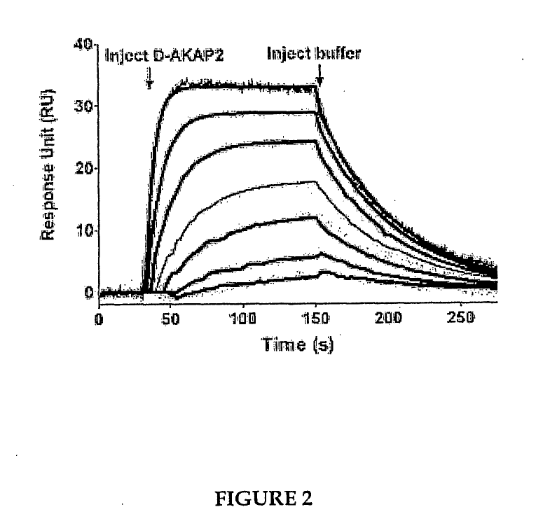

To evaluate the functional properties of this protein, binding of D-AKAP2 to the RIIα isoform of PKA was examined using surface plasmon resonance (Herberg et al., J. Mol. Biol. 298:329-339, 2000). RIIα (cAMP free) was immobilized to cAMP that was attached covalently to a sensor chip. D-AKAP2 was injected over the surface at varying concentrations and the affinity determined by fitting the curves to a 1:1 binding model (see FIG. 2). D-AKAP2 bound tightly to RIIa with on and off rates of 1.3×10⁶ M⁻¹s⁻¹ and 18×10⁻³ s⁻¹, respectively, resulting in a K_{d} of 14 (±2) nM. In contrast, the dissociation constant for the AKB domain alone was much lower at 2 nM, suggesting that the AKB domain was not fully accessible in the intact, soluble protein.

v. Tuning of D-AKAP2 Proteolytic Fragmentation.

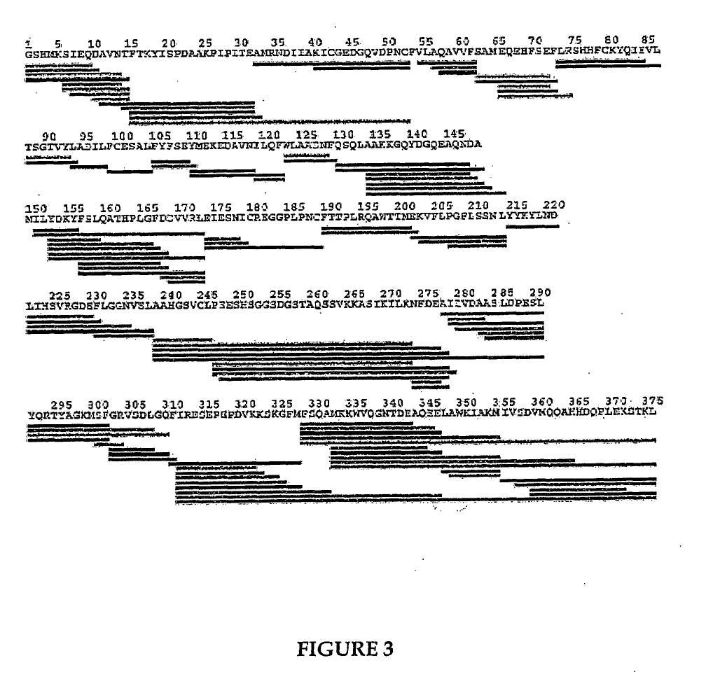

Prior to studying the hydrogen-exchanged samples, digestion conditions that produced D-AKAP2 fragments of optimal size and distribution for exchange analysis were established. Minimal back exchange and optimal pepsin digestion for D-AKAP2 (4.7 μM) were obtained by diluting one part of the deuterated sample with one and a half parts of quench solution (3.2 M GuHCl in 0.8% (v/v) formic acid, pH 2.0). The quenched sample was then run over immobilized pepsin (66 μl bed volume) at a flow rate of 200 μl/minute, resulting in a digestion duration of 20 seconds. These conditions generated 119 peptides covering the entire amino acid sequence of D-AKAP2 (see FIG. 3). Forty of the identified peptides, representing 99% of the entire sequence (373 out of 375 amino acid residues), were of good quality for DXMS analysis (bold lines in FIG. 3). Since both the amino group of the first amino acid residue and the amide hydrogen atom of the second amino acid residue exchange too rapidly to retain deuterons during the experiment (Bai, supra) the total number of amide hydrogens followed by DXMS was 302 out of 362 non-proline residues (83%).

vi. Deuterium On-Exchange of D-AKAP2 at 0° C.

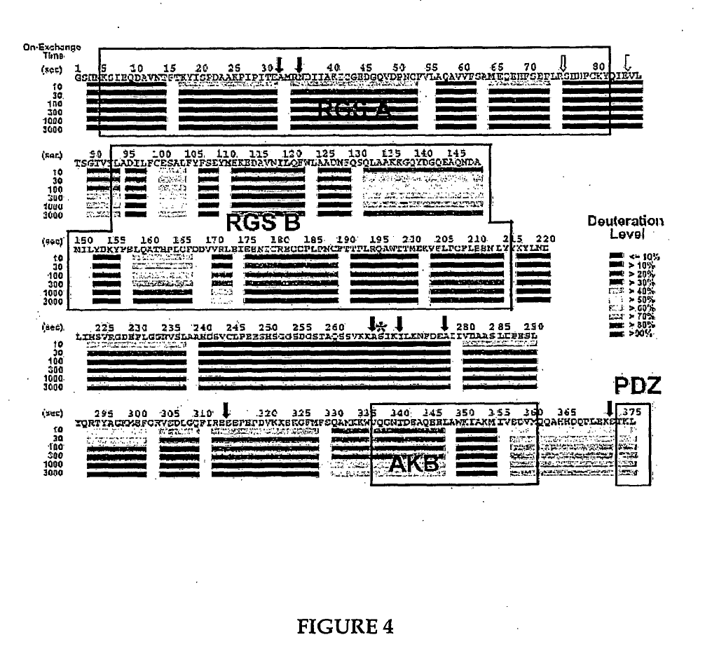

D-AKAP2 was incubated in deuterated buffer for 10-3000 seconds at 0° C. and quenched with a low-pH buffer to slow the back-exchange rate during analysis. The extent of deuterium incorporation was determined by mass spectrometry and reported as percentage deuteration, as described herein (see FIG. 4). The deuterium exchange profile divided the protein into four main regions. The first region (G1-C52) was fast exchanging, with all three peptides heavily deuterated. This region contained part of the RGS A domain and was most likely disordered due to the N-terminal truncation of this protein used in this study. The second region (A56-D220) included the rest of the RGS A domain and the entire RGS B domain. This region showed the greatest area of protection and was presumably the most structured region of the protein. Computational modeling was applied to further investigate the structure of this region (see below). The third region (H223-M327) was heavily exchanged and included a putative PKA phosphorylation site. The final region (Q330-L375), located at the C terminus, was exchanging relatively slowly and included the AKB and PDZ domains.

The overall exchange pattern of D-AKAP2 was consistent with the limited proteolysis results. Seven cleavage sites (black arrows in FIG. 4) were identified after one hour of limited proteolysis and all of them were located in fast-exchanged regions. Two cleavage sites at R74-S75 and E84-V85 (open arrows in FIG. 4) were in highly protected regions. However, they were only detected after 24 hours of digestion, suggesting that initial protease digestion could have destabilized this region.

vii. Modeled RGS Domain.

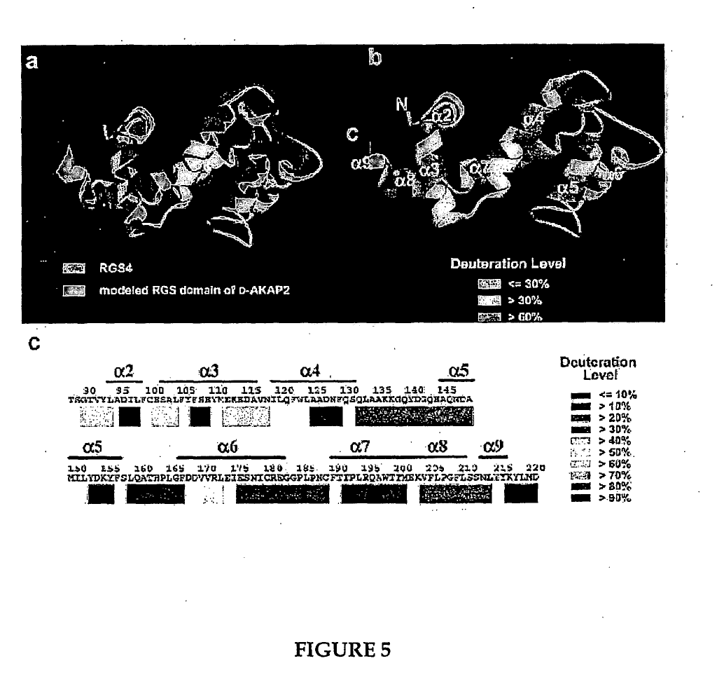

In order to provide some structural insight into the deuteration levels observed, the sequence for the RGS B domain of D-AKAP2 was threaded onto the crystal structure of RGS4. Residues L93-Y214 were identified as the RGS domain boundaries for D-AKAP2 and were 22% identical (41% similar) to RGS4 (see FIG. 1B). Helices 2 through 9 of RGS4 were used as a template for structural modeling. The region corresponding to helix 1 did not share significant homology between the two sequences and therefore was not modeled. The residues comprising the hydrophobic core of the RGS domain as described in Tesmer et al. (supra) were highly conserved in D-AKAP2, suggesting a potential to fold into an RGS domain (see FIG. 1B). The majority of the residues in the modeled RGS domain occupied the most favored regions of the Ramachandran plot. Only 6.6% of the residues were in the disallowed region, similar to the initial RGS4 template that had 6.4% in the disallowed region. The structural alignment for the α-carbon atoms of the RGS4 template and the model RGS domain of D-AKAP2 were within 0.7 Å, with a Z-score of 6.0, indicating a high level of structural similarity between the RGS4 template and the modeled RGS domain (see FIG. 5A). The structure of the modeled RGS domain deviated the most from RGS4 in the loop region between helices 4-5 and 6-7 of the four-helix bundle of this domain. For the modeled RGS domain there was a seven residue insertion between helices 4 and 5 (see FIG. 1B), which extended the loop, making this region difficult to model.

Deuterium exchange at 3000 seconds and 0° C. was mapped onto the modeled RGS domain. The most protected regions mapped to the α-helices and the more exchanged regions mapped to the connecting loops and turns (see FIGS. 5B and 5C). Seven peptides in the RGS B domain remained less than 30% deuterated after 3000 seconds on-exchange at 0° C. and were mapped to α-helices 2 (peptide 93-97), 3 (104-108), 4 (122-128), 5 (150-155), 7 (189-200), 8 (201-213) and the C terminus of the RGS domain near helix 9 (214-220). Consistent with this, all but two (L164 and F166) of the hydrophobic core residues that were conserved throughout the RGS family were located within or near these highly protected regions (see FIG. 5C). Residues 168-172 in helix 6 showed deuteration levels about 50%, indicating that this helix was more flexible and/or solvent accessible than the other helices of the RGS domain. The turn between helices 2 and 3 and the extended loop between helices 4 and 5, which contained the seven residue insertion, were approximately 70% and 80% deuterated, respectively. The most exchanged peptide covered the loop between helices 5 and 6 with over 90% deuteration. The Gα interaction surface for RGS domains includes this highly solvent-accessible region at the base of the four-helix bundle.

viii. AKB Domain.

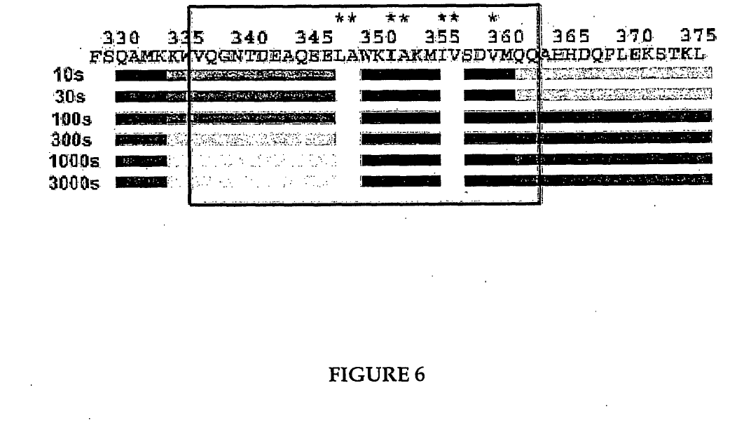

In addition to the putative RGS domain, the AKB domain was also well protected from deuterium exchange at 0° C. A 27 residue peptide (box in FIG. 4) binds to the regulatory subunit of PKA with high affinity. A high level of protection from exchange was observed within this region from L349-M354. To further sub-localize the slowly exchanging regions near the predicted amphipathic helix (L347-M360), the deuteration levels of overlapping peptides were compared. First, the deuteration percentages of peptides 355-375 and 359-375 were converted to the numbers of deuterons incorporated by multiplying the deuteration percentage by the maximum deuterium incorporation (maxD). The deuteron difference of the two peptides was divided by maxD of residues S357-M360 to give the deuteration percentage of the residues. The results indicated that residues S357-M360 were not deuterated in the time window examined, extending the protected region to residues W349-M360 (see FIG. 6). Similar analysis for peptide 328-346 and peptide 334-346 revealed a well-protected region upstream of the PKA binding site.

The high level of protection of the AKB domain was somewhat unexpected, since this surface interacts with the regulatory subunit of PKA. To investigate if the protection was due to secondary or tertiary structural effects, a peptide containing only the C-terminal 40 residues was examined. After deuteration for 10 seconds at 22° C. the entire peptide was fully deuterated, even though the CD spectrum of this peptide indicated that the peptide could form secondary structure under the conditions used for the deuterium exchange experiments. This suggests that, in the context of the larger D-AKAP2 fragment, the AKB domain is further stabilized by tertiary interactions.

ix. The Comparison of Deuteration Levels at 0° C. and 22° C.

It was important to establish that the results at 0° C. did not differ significantly from those obtained at 22° C., where most of the binding studies were performed. According to the Arrhenius equation, the exchange rate would increase by approximately a factor of 10 at 22° C. from that at 0° C.(k(T)=A exp (−Eₐ/RT); Eₐ for base-catalyzed amide hydrogen exchange reaction is 17.4 kcal mol⁻¹: 1 cal=4.184 J) (Bai et al., supra). There was no evidence of a major conformational change of the protein in going from 0° C. to 22° C. All peptides that were highly protected at 0° C., remained highly protected at 22° C.; all peptides that were heavily exchanged remained heavily exchanged, and all moderately exchanged peptides increased their deuteration levels continuously at longer exchange times. Only two (87-93 and 98-103) of the 15 moderately exchanging peptides showed 10% or more increase in deuteration levels over what would be predicted due to the higher temperature (see Table 2), indicating that the conformational change due to temperature was very small (Zhang and Smith, supra).

TABLE 2
Deuteration Level Increase from 0° C. to 22° C.

| Peptide | Increase (%)* |
|---|---|
| 87-93 | 13 |
| 93-97 | 7 |
| 98-103 | 10 |
| 109-117 | 9 |
| 129-148 | 8 |
| 156-166 | 2 |
| 168-172 | 9 |
| 173-188 | 9 |
| 189-200 | 1 |
| 201-213 | 3 |
| 214-220 | 0 |
| 291-301 | 2 |
| 310-327 | −1 |
| 328-346 | 3 |
| 355-375 | 7 |

*Increase was the average of deuteration level increases from 0° C. to 22° C. at four equivalent exchange times (100 seconds at 0° C. versus 10 seconds at 22° C., 300 seconds at 0° C. versus 30 seconds at 22° C., 1000 seconds at 0° C. versus 100 sec at 22° C., and 3000 seconds at 0° C. versus 300 sec at 22° C.).

C. Discussion of DXMS Analysis of D-AKAP2.

The discovery that AKAPS are scaffold proteins is introducing a new dimension to the understanding of cell signaling. While information is unfolding regarding specific binding motifs, very little is known about the overall structure and dynamics of these scaffolds. The present invention provides a first glimpse of a scaffold protein using limited proteolysis and DXMS to explore the dynamic properties of these proteins and their interacting partners.

The C-terminal forty residues of D-AKAP2 were initially identified in a genomic screen as interacting with the regulatory (R) subunit isoforms of PKA (Huang et al., supra). After subsequent cloning of the full-length gene (coding for 662 amino add residues), the protein was predicted to have multiple domains including two putative RGS domains, a PKA binding site and a PDZ binding motif (Wang et al., supra). The examples provided herein show the expression of a truncation of D-AKAP2 (residues 291-662) and demonstrate that this protein binds tightly to the type II regulatory sub-unit of PKA using DXMS analysis. The domain boundaries were mapped using traditional limited proteolysis and further refined using DXMS, providing a higher resolution of detail and an insight into the intra-domain dynamics and function of D-AKAP2.

The two tandem RGS domains suggested the potential of D-AKAP2 to coordinate upstream heterotrimeric G proteins signaling with downstream PKA signaling. The examples presented herein show that RGS B does assume an RGS-like fold. The sequence containing this region modeled well as an RGS domain. The CD spectrum of D-AKAP2 was suggestive of a mostly α-helical protein. Moreover, hydrophobic residues that comprised the core of the fold were conserved in D-AKAP2 and mapped to the highly protected, helical regions of RGS B. In addition, the flexible and/or solvent-accessible regions, determined by DXMS analysis, mapped to the loops and turns, consistent with the structural model. The turn between helices 5 and 6 was the most solvent accessible region of RGS B. Interestingly, this region in RGS4 makes contacts with the switch regions of Gα, stabilizing the transition state and enhancing the GTPase activity of Gα (Zhang and Smith, supra).

Despite the evidence that this region folds into an RGS domain, no Gα binding partner has been identified. Lack of a traditional Gα binding partner may be explained by several notable differences between the RGS domains of D-AKAP2 and other RGS proteins. In a phylogenetic classification of mammalian RGS proteins, D-AKAP2 did not contain significant homology to other known members in the family (Zheng et al., Trends Biochem. Sci. 24:411414, 1999). The residues important for interaction with the switch regions of the Gα subunit are not conserved in D-AKAP2. In addition D-AKAP2 is the only mammalian protein identified that contains two RGS domains one of which is a “split” RGS domain. The presence of two RGS domains and the split nature of the N-terminal RGS domain are properties recognized only by lower eukaryotes such as Caenorhabditis elegans and Saccharomyces cerevisiae. Sst2 from S. cerevisiae, the first RGS domain identified, contains large insertions of amino acid residues between helices 1 and 2; 4 and 5; and 6 and 7 of the RGS domain (Zhang and Smith, supra). It is unclear how the presence of the tandem RGS domains and the large 124 residue insert between helices 4 and 5 of the RGS A domain of D-AKAP2 would affect the RGS fold or the specificity of its interaction partners. The extended region may provide additional interaction sites for binding non-traditional partners.

Lack of a Gα binding partner for an RGS domain is not unusual. Axin, a negative regulator of the Wnt growth-factor signaling pathway, contains an RGS domain and has not been shown to interact with any Gα subunits. However, it does use its RGS domain to interact directly with adenomatous polyposis coli (APC), a tumor suppressor protein (Spink et al., EMBO J. 19:2270-2279, 2000). D-AKAP2 may use similar alternative binding surfaces.

In addition to providing a scaffold for binding other proteins, the multi-domain organization of D-AKAP2 altered the binding affinity to PKA. The low level of protection of the AKB domain alone compared with the intact protein, suggested that, in the absence of PKA, this region was stabilized by intramolecular interactions. Residues 347-360 comprise the PKA binding surface and included the most protected region at the C terminus of the intact protein.

The reduced binding affinity observed for the binding of the intact protein to the regulatory subunit compared with the AKB domain alone was consistent with the model that a conformational change was required to form a tight complex with PKA. In vitro studies have shown that PKA can phosphorylate D-AKAP2 at S267, which is located in a highly solvent-accessible region between the putative RGS domain and the AKB domain. The effects of phoshorylation on the binding affinity to PKA and the domain organization are currently being determined, but it is possible that a phosphorylation feedback mechanism may be important to modulate the accessibility of this region to PKA.

The structures of two well-characterized AKB domains from AKAP79, an AKAP involved in neuronal signaling, and human thyroid anchoring protein HT31 have been solved by NMR in complex with the D/D domain of RIIa (Newlon et al., EMBO J. 20:1651-1652, 2001). Both AKBs bind similarly to the regulatory subunit with the hydrophobic face of the helix interacting with a hydrophobic groove on the surface of the regulatory subunit D/D domain. This raises the issue of how binding specificity is achieved for AKAPs involved in very different signaling pathways, and suggests that domains outside of the AKB could modify PKA binding affinity.

In contrast to the high level of protection of the AKB domain, the adjacent C-terminal PDZ-binding motif was located in a very solvent-accessible region. The accessibility of this region may be important for recognition by PDZ domain-containing proteins, thereby serving as a targeting domain for D-AKAP2. It will be important to identify interacting PDZ domains and to determine if binding of PDZ domains influences PKA binding and the accessibility of this region.

The present invention provides improved methods utilizing amide hydrogen exchange liquid chromatography-mass spectroscopic methods amenable to a high-throughput, high-resolution applications well suited to the study of isolated proteins as well as protein-protein binding interactions. Some of the improvements developed in the methods of the present invention have focused on the protein chemistry employed, such as, for example, improved quench-compatible methods using denaturants and immobilized pepsin in tandem with mass spectrometry. The combination of these improvements has produced comprehensive amide hydrogen coverage of D-AKAP2 and has allowed mapping of the domain organization of this protein without any prior knowledge of its structure. In this study of D-AKAP2, 99% of the protein sequence was covered by pepsin generated peptide fragments of high quality and 83% of the peptide linkage (including proline residues) could be followed. This high coverage enabled investigation of the overall structure of D-AKAP2. Sub-localization of deuterons incorporated was demonstrated at the C-terminal AKB domain. The subtraction of the deuterium incorporations of analogous peptides expanded the highly protected region, indicating that the accuracy of the invention methods is high enough to obtain higher resolution information via subtraction of two peptides.

The present invention thus provides novel tools for large-scale proteomic analysis, allowing high-throughput techniques that provide information on protein structure and function. Several global initiatives have focused on structural determination by high-resolution techniques such as NMR and X-ray crystallography (Stevens et al., Science 294:89-92, 2001). While these methods are invaluable in understanding the 3D structure of a protein, they need to be complemented with techniques that examine the dynamic nature of proteins in solution and the assembly and disassembly of proteins into large multi-functional complexes. The methods of the present invention utilizing amide hydrogen exchange analysis are uniquely valuable in this regard. The examples provided herein show that enhanced amide hydrogen exchange methodologies, DXMS, can be used to rapidly gain high-resolution and comprehensive information on the folded regions of a protein. This approach, in combination with sequence and structural modeling can provide unique insights into the structure and function of a protein of interest.

### EXAMPLE 7

### Automated DXMS Analysis

A. Automated DXMS Apparatus.

Equipment configuration for these studies consisted of high pressure switching valves (Rheodyne 7010) connected to pumps and other components with PEEK tubing (Upchurch Scientific); a Spectraphysics AS3000 autosampler (Thermo Finnigan LLC, San Jose, Calif.) in which all but twenty of the sample vial positions were filled with powdered dry ice; a pepsin-20AL column (66 μl bed volume, porcine pepsin (Sigma) coupled to 20AL support material per the manufacturer's instructions (PerSeptive Biosystems); a C18 column (1×50 mm, Vydac catalog no. 218MS5105). Four HPLC pumps (Shimadzu LC-10AD, operated by a Shimadzu SCL-10A pump controller); one pump delivered 0.05% aqueous TFA to push samples through the pepsin column; and another delivered the same buffer to backflush the pepsin column after sample digestion. Two additional pumps delivered solvents for HPLC column gradient elution (pump A; 0.05% aqueous TFA and pump B; 80% acetonitrile, 20% water, 0.01% TFA). Inline filters (0.5 μm Upchurch catalog no. A.430) were placed on each side of the pepsin column and just before the C18 column (Vydac prefilter, catalog no. CPF 10) to minimize column fouling and carryover of aggregated material. To provide precise temperature control, valves, tubing, columns and autosampler were contained within a refrigerator maintained at 2.8° C., with columns also immersed in melting ice. Mass spectrometric analyses were carried out with a Finnigan LCQ electrospray ion trap type mass spectrometer (Thermo Finnigan) with capillary temperature at 200° C.

B. General Operational Procedure.

Sets of autosampler vials containing quenched, functionally deuterated samples (50 μl, stored at −80° C.) were placed in the dry-ice-containing sample basin of the autosampler. The samples were held there at dry ice temperature until individually melted by the autosampler at 2-3° C. over 1.5 minutes, utilizing its sample preparation features, and then injected (45 μl) onto the pepsin column. The samples were pumped through the pepsin column (0.05% TFA at 100 μl/min) with contemporaneous collection of digestion products on the C18 HPLC column. The digested peptides were separated by a linear acetonitrile gradient (5-45% B/10 minutes; 50 μl/min; solvent A, 0.05% TFA; solvent B, 80% acetonitrile, 20% water, 0.01% TFA). The mass spectrometer acquired spectra on the effluent in either MS1 profile mode, or data-dependent MS2 mode.

C. Deuterium Exchange Experiments.

All exchange mixtures for Csk contained the following: 9.8 nM Csk, 18 mM Mops, 45 mM NaCl, and 0.9 mM DTT. Final pH was 7.0, and final percentage of D₂O was 90%. Exchange experiments in the presence of nucleotides included 1 mM ADP and 2 mM AMP-PNP with 11 mM MgCl₂ and 12 mM MgCl₂, respectively. Csk was pre-equilibrated with the respective nucleotides in H₂O before starting the deuterium exchange by diluting into D₂O. The D₂O mixture was prepared as follow. Appropriate amounts of Mops, NaCl, and DTT were dissolved in D₂O and mixed with deuterated solutions of nucleotide and MgCl₂. The final mixture contained 20 mM Mops, 50 mM NaCl, and 1 mM DTT, and either 1 mM ADP and 11 mM MgCl₂, or 2 mM AMP-PNP and 12 mM MgCl₂. The H₂O mixtures contained 98 nM Csk, and either 1 mM ADP and 11 mM MgCl₂, or 2 mM AMP-PNP and 12 mM MgCl₂. Deuterium exchange was initiated by the addition of 20 μl of the H₂O solutions to 180 μl of the D₂O solutions. The solutions were incubated on ice. At various times, 20 μl aliquots were removed and added to an ice-cold tube containing 30 μl of 0.8% formic acid with 0.8 M GuHCl. This brought the Csk solution down to pH 2.5 and quenched the deuterium exchange. The quenched samples were frozen in dry ice and stored at −80° C. prior to ESI MS analysis. Freeze-thawing did not significantly affect the extent of deuterium incorporation.

D. In- and Back-Exchange Controls.

Controls were performed as previously described (Andersen et al., J. Biol. Chem. 276:14204-14211, 2001). Briefly, non-deuterated Csk was used as a control for in-exchange of deuterium. The sample was prepared under the same quench conditions as above, but in the absence of D₂O. This sample served as zero time point. The back-exchange was determined by incubating Csk in D₂O containing 0.5% formic acid overnight at 25° C. This allows for complete exchange of backbone amide protons for deuterium. To determine the amount of label lost during the experiment (i.e., back-exchange) the deuterated sample was treated with quench solution and subjected to ESI MS analysis. This control provides, for each peptide, the maximal experimental mass that relates to a fully exchanged peptide. To confirm that the conditions employed were sufficient to fully (equilibrium) deuterate the intact protein, Csk was first proteolysed on the pepsin column, as above, effluent peptides collected in bulk, dried, and peptides taken up and incubated in D₂O containing 0.5% formic add overnight at 25° C. Subsequent LCMS analysis of these deuterated peptides was performed as for the intact Csk except that the pepsin column was bypassed to avoid re-digestion. The deuterium content of corresponding peptides was identical with the two methods of deuteration.

### EXAMPLE 8

### DXMS Analysis Used to Elucidate Phosphorylation-Driven Motions in the COOH-Terminal Src Kinase Csk

A. Background.

The Src family of nonreceptor protein tyrosine kinases (nrPTKs) bind to receptor protein tyrosine kinases (PTKs) where they phosphorylate down-stream protein targets associated with discrete signaling pathways (Superti-Furga and Courtneidge, Bioessays: 17:321-330, 1995; Neet and Hunter, Genes Cells 1:147-169, 1996; and Tatosyan and Mizenina, Biochemistry 64:49-58, 2000). While the Src enzymes comprise a large subfamily of nrPTKs, all are regulated through a single nrPTK, Csk (COOH terminal Src kinase). Csk down-regulates kinase activity by phosphorylating a single tyrosine residue in the C-terminus of the Src enzymes (Okada et al., J. Biol. Chem. 266:24249-24252, 1991; and Bergman et al., EMBO J. 11:2919-2924, 1992). Owing to this premier regulatory function, Csk has direct effects on many biological functions including T cell activation, neuronal development, cytoskeletal organization, and cell cycle control (Inomata et al., J. Biochem. 116:386-392, 1994; Latour and Veillette, Curr. Opin. Immunol. 13:299-306, 2001; Taylor and Shalloway, Bioessays 18:9-11, 1996; and Zenner et al., Bioessays 17:967-975, 1995). The general significance of Csk is also evident in the lethality of gene knockouts in mice (Hamaguchi et al., Biochem. Biophys. Res. Commun. 224:172-179, 1996). Csk contains three structural components essential for in vivo function: a tyrosine kinase domain, an SH2 domain, and an SH3 domain. The structure of the kinase domain, solved by x-ray diffraction, adopts a standard kinase fold with typical nucleotide and substrate binding lobes (Lamers et al., J. Mol. Biol. 285:713-715, 1999). Unlike Src family nrPTKs, Csk is not upregulated through activation loop phosphorylation. The x-ray structures for α-Src, illustrate that the C-terminus is phosphorylated and interacts tightly with the SH2 domain (Sicheri et al., Nature 385:602-609, 1997; Williams et al., J. Mol. Biol. 274:757-775, 1997; and Xu et al., Nature 385:595-602, 1997). In Csk, no such interaction is possible owing to the absence of a phosphorylatable sequence in the C-terminus. As revealed by x-ray diffraction studies, this generates a unique domain organization where the SH2 domain interacts with the small lobe of the kinase core in Csk rather than the large lobe as in c-Src (Ogawa et al., J. Biol. Chem. 277:14351-14354, 2002).

Understanding the conformational nature of protein kinases in solution is important for evaluating function since it has been shown that slow structural movements can limit substrate phosphorylation. The first pre-steady-state kinetic studies applied to a protein kinase, cAMP-dependent protein kinase (PKA), revealed that slow conformational changes associated with nucleotide binding and release limit catalytic cycling (Grant and Adams, Biochemistry 35:2022-2029, 1996; Shaffer and Adams, Biochemistry 38:12072-12079, 1999; and Shaffer and Adams, Biochemistry 38:5572-5581, 1999). Since these early investigations, two other protein kinases have been studied using fast mixing kinetic techniques. While the tyrosine kinases Her-2 and Csk rapidly phosphorylate substrates in the active site, rate-limiting events in the catalytic cycle are associated with slow conformational changes linked to ADP release (Shaffer and Adams, Biochemistry 40:11149-11155, 2001; and Jan et al., Biochemistry 39:9786-9803, 2000). Although more kinetic investigations are clearly needed for a broad assessment of function, the detailed investigations into these three protein kinases reveal a common motif for activity regulation. Once ATP and the substrate are appropriately oriented in the active site, phosphoryl transfer occurs with little impediment. In contrast, the regeneration of this active complex occurs partly through slow conformational changes that appear to be linked to ADP release.

Amide hydrogen exchange techniques have proven to be increasingly powerful tools by which protein dynamics, structure and function can be probed. Deuterium exchange methodologies coupled with either MALDI or Electrospray (ESI) Mass Spectrometry, presently provide one of the most effective approaches to study proteins larger than 30 kDa in size. Proteolytic and/or collision-induced dissociation fragmentation methods allow exchange behavior to be mapped to subregions of the protein. In a previous study using such techniques, it was demonstrated that ADP binding induces long-range structural changes in the catalytic subunit of PKA (Andersen et al., J. Biol. Chem. 276:14202-14211, 20010. Two of these regions encompass critical loops in the active site, as expected, whereas two other regions are distally located. These regions encompass the C-terminus and helix αC. Based on crystallographic evidence, the latter secondary structural element is known to move in phosphorylation- and subunit-dependent manners in several other protein kinases (Jeffrey et al., Nature 376:313-320, 1995; and Hubbard, EMBO J. 16:5572-5581, 1997). The exciting inference derived from these solution studies is that long-range perturbations may be coupled to slow conformational changes detected in the kinetic mechanism for PKA (Shaffer and Adams, supra). Thus, a tangible link between catalytic function and solution structure may now be established.

Prior kinetic studies have shown that conformational changes associated with ADP release provide a regulatory mode for substrate phosphorylation in the nrPTK, Csk. In the study presented herein, the effects of nucleotide binding on the solution conformation of Csk were monitored with DXMS. Earlier amide hydrogen exchange techniques have been successfully applied to two protein kinases, to date; PKA (Andersen et al., supra) and ERK2 (Resing and Ahn, Meth. Enzymol. 283:29-44, 1997; Resing and Ahn, Biochemistry 37:463-475, 1998; and Resing et al., J. Am. Soc. Mass Spectrometry 10:685-702, 1999). Both kinases are structurally simple being composed primarily of kinase domains. In comparison, Csk has more elaborate domain structure with the tyrosine kinase domain flanked by two noncatalytic SH2 and SH3 domains. These domains are thought to limit movements in the kinase core, impair nucleotide access and release and diminish catalytic activity in the structurally related c-Src (Sicheri et al., supra). In this study DXMS demonstrates that nucleotide binding induces long-range changes in the structure of Csk. A comparison of the ATP (AMPPNP)- and ADP-forms reveals unique structural changes induced by the γ phosphate of the nucleotide. These structural effects ramify not only throughout the small and large lobes of the kinase domain but also modify intra-domain dynamics.

B. Results of DXMS Analysis of Csk.

i. Tuning of Csk Proteolytic Fragmentation.

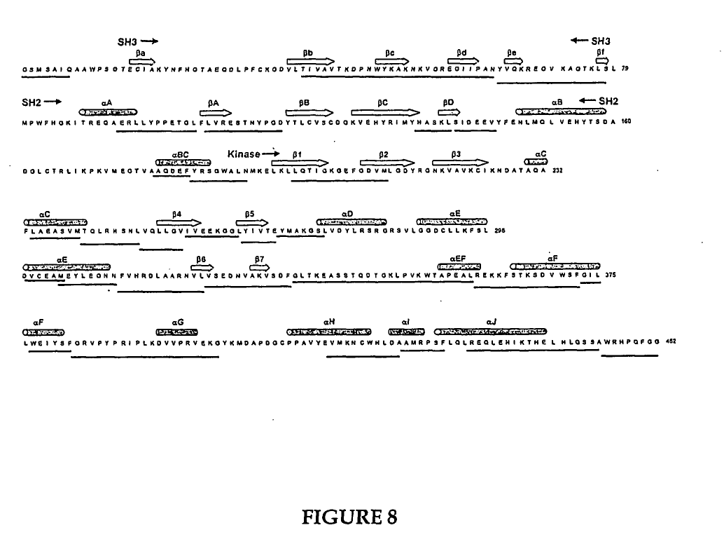

Prior to studying the hydrogen exchanged samples, digestion conditions that produced Csk fragments of optimal size and distribution for exchange analysis were established as described earlier. Minima back-exchange and optimal pepsin digestion for Csk were obtained by diluting one part of the deuterated sample with one and a half parts of quench solution (0.8 M GuHCl in 0.8% formic acid). The quenched sample was then run over immobilized pepsin (66 μl bed volume) at a flow rate of 100 μl/min, resulting in digestion duration of 40 seconds. These conditions generated 28 high quality peptides covering 63% of the Csk sequence of (see FIG. 8). Since both the amino group of the first amino acid and the amide hydrogen of the second amino add exchange too rapidly to retain deuterons during the experiment, the total number of amide hydrogens followed by DXMS was 223 out of 444 non-proline residues (50%).

ii. Deuterium Incorporation into the Proteolytic Fragment Probes.

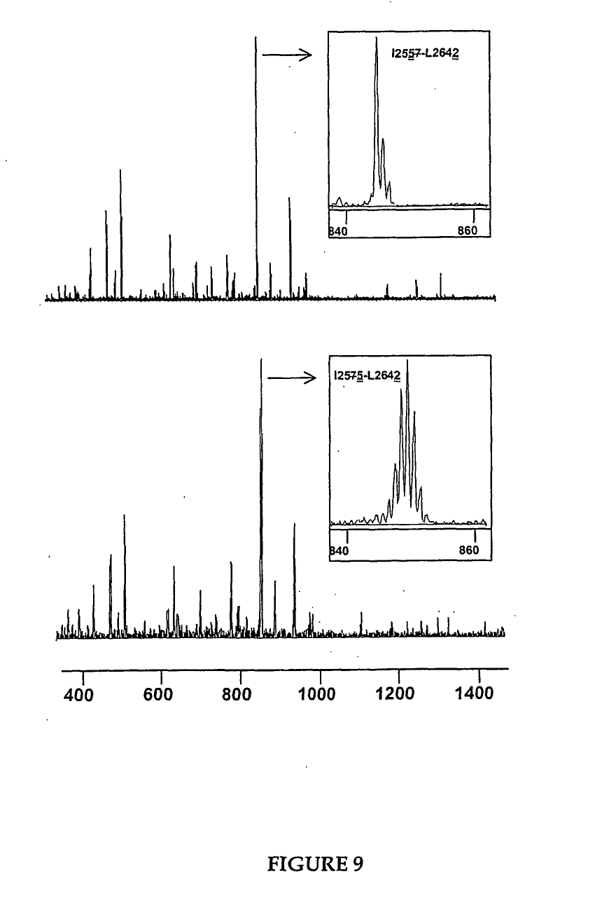

The incorporation of deuterium from solvent D₂O can be monitored using DXMS. FIG. 9 shows the ESI spectra of proteolyzed Csk before and subsequent to dilution into D₂O. The insets in this figure highlight one specific peptide fragment, 1255-L262. This probe appears as a cluster of peaks owing to the natural isotopic distribution of the atoms in the peptide. After 3 hours of incubation in D₂O, the envelope of peaks for the probes increases in overall mass and complexity. The centroids for these two clusters are used to determine the mass of the probe both before and after incubation with solvent deuterium.

iii. Effects of Nucleotide Binding on Deuterium In-Exchange.

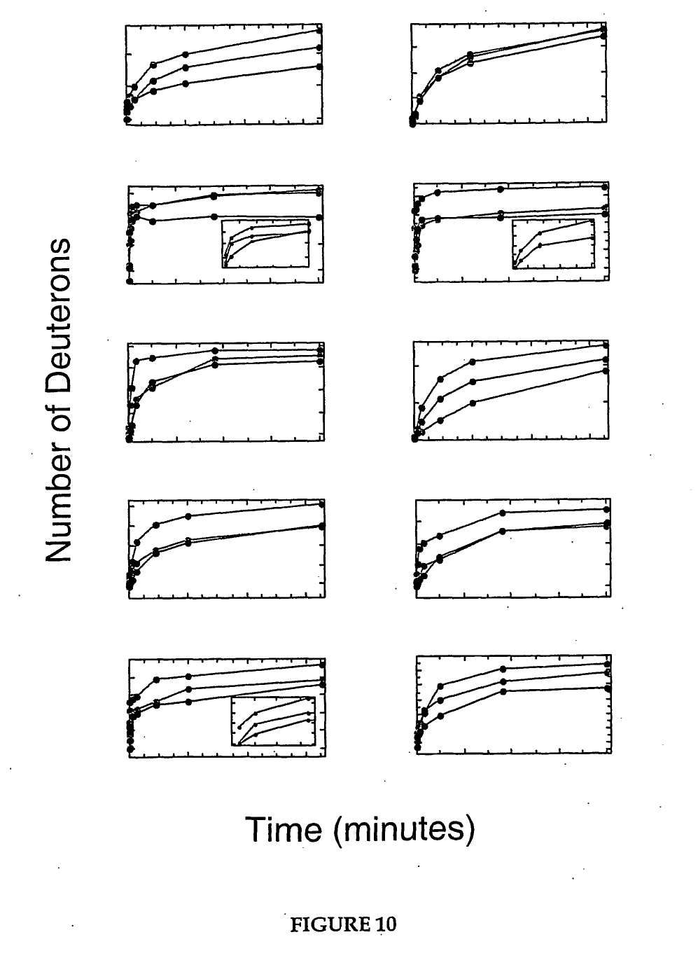

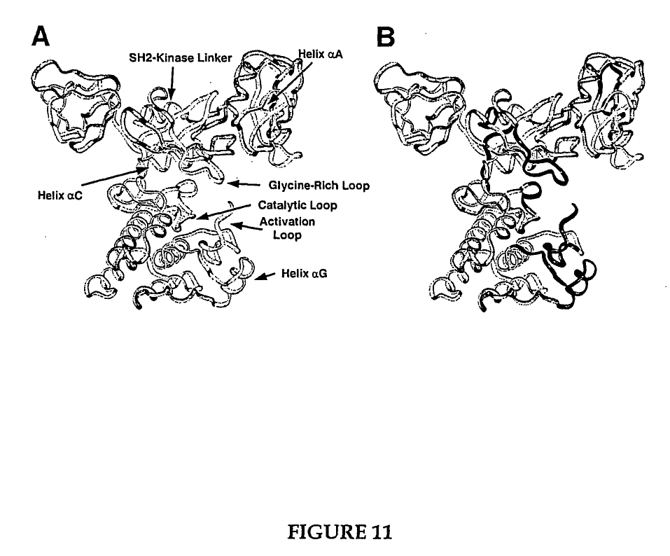

The average mass of each peptide was elucidated by integrating over the full envelope of peaks. To quantify the extent of deuterium incorporation at various time periods, the mass of each probe was converted to a number of in-exchanged deuterons using Equation 1. The in- and back-exchange controls set the zero and infinite time points for D (t). Each peptide fragment is unique with different numbers of exchangeable protons and different intrinsic exchange rates. This method detects total mass changes for each probe without defining the priority of amide exchange within each probe. Deuterium incorporation into the Csk probes was followed as a function of time in the absence and presence of two nucleotides: AMPPNP and ADP. FIG. 10 displays the time-dependent incorporation of deuterium into several typical probes. In some cases (e.g., S139-V144), the presence of either nucleotide has no effect on the incorporation of deuterium over the experimental time frame (10 seconds to 1200 minutes). These probes are considered non-protected by the ligand over the exchange times studied. By comparison, the rate of deuterium incorporation into other probes is impaired by the ligand. These probes are considered protected by the nucleotide. In no cases did the nucleotide increases the rate of deuterium incorporation compared to the apo-enzyme. The definition of whether a probe is protected by the nucleotide over the experimental time frame depends on the accuracy of the mass measurement in the ESI spectrometer. For the set of probes used in these studies kinetic traces that differ by more than 0.5 deuterons in mass at a minimum of two points are defined as experimentally different. Using these criteria, we have identified a number of probes whose deuteration rate is impaired by the presence of the nucleotide (see FIG. 10). FIG. 11B shows the probes that are protected from exchange by AMPPNP. Furthermore, we have identified probes that are differentially protected by AMPPNP and ADP. These probes are displayed onto the Csk structure in FIG. 11B.

C. Discussion of DXMS Analysis of Csk.

Since the crystallographic solution of the first protein kinase structure approximately one decade ago, it has become apparent that this enzyme family undergoes structural changes that are linked to activity regulation. For example, many protein kinases have been crystallized in both “open” and “dosed” forms that differ by domain rotations (Johnson et al., Cell 85:149-158, 1996). Other protein kinases that are regulated through phosphorylation and protein binding display large movements in loop and helical regions upon activation. The cyclin-dependent protein kinase, cdk2, and the insulin receptor kinase undergo large changes in helix αC and the activation loop when a cyclin binds in the former case and upon phosphorylation in the latter case (Jeffrey et al., supra; Hubbard, supra; and Hubbard et al., Nature 372:748-754, 1994). It has also been demonstrated that discrete structural changes partially or fully limit substrate processing in several protein kinases based on pre-steady-state kinetic measurements (Shaffer et al., supra; Shaffer and Adams, supra; and Jan et al., supra). In this example DXMS is employed to probe the solution conformation of the nonreceptor PTK, Csk. Previous kinetic studies have shown that slow conformational changes limit ADP release. To address whether Csk adopts any unique structural states that may be important for regulation, the solution conformation of the full-length enzyme was studied in the absence and presence of the product, ADP, and a nonhydrolyzable ATP analog, AMPPNP.

i. Effects in the Kinase Domain.

While two regions in the active site of Csk that are expected to interact with ATP (catalytic & glycine-rich loops; see FIG. 11) are highly protected from deuterium incorporation in the presence of AMPPNP, several regions outside the active site are also protected by nucleotide. For example, the probe encompassing helix αC (L234-M240, see FIG. 10) is protected by AMPPNP by as much as 2 deuterons over intermediate exchange time frames. Such protection has also been observed in PKA upon ADP binding. This helix does not make any direct contacts with the nucleotide but rather contains a conserved residue (Glu-236, Glu-91 in PKA) that forms a salt bridge with another conserved residue (Lys-222, Lys-72 in PKA). Lys-72 in PKA has been shown to form interactions with the αβ phosphates of ATP suggesting that Lys-222 could serve a similar function in Csk. The electrostatic dyad between these two residues (Glu-Lys) is conserved in the enzyme family and also appears to be disrupted in several down-regulated protein kinases. For example, this disruption in the InRK and cdk2 is coordinately linked to movements in helix αC (Jeffrey et al., supra; and Hubbard, supra). While these motions occur upon cyclin binding for cdk2 and upon activation loop phosphorylation for the InRK, the protection observed in our H-D exchange experiments suggest that movements in this helix may be induced solely by nucleotide binding.

The association of nucleotide with Csk has profound effects on regions in the large lobe of the kinase domain. Protection of a probe containing the activation loop (G334-L358, see FIGS. 10 and 11) suggests that this region is affected by nucleotide binding. While this loop is not expected to contact ATP directly, a conserved aspartate (Asp-184 in PKA, Asp-332 in Csk) preceding the activation loop chelates the essential, activating Mg²⁺ which stabilizes the γ phosphate of ATP. It is conceivable that protection in the activation loop upon AMPPNP binding in Csk may reflect conformational changes linked to movements in this preceding structural element. The binding of nucleotide to Csk has further effects on the large lobe of the kinase domain. Most notably, the probe containing helix αG is protected from deuterium incorporation by AMPPNP (see FIG. 11). This helix makes no direct contact with the nucleotide and is even further removed from the active site than helix αC and the activation loop. While it is not clear how AMPPNP can transmit such long-range effects across the kinase domain, space-filling models illustrate that the activation loop packs on top of helix G (Jeffrey et al., supra). Such findings suggest that the binding of the nucleotide has pervasive effects on the kinase domain which ramify from their origins in the small ATP-binding lobe down to the larger substrate-binding lobe.

ii. Inter-Domain Cross-Talk.

Prior kinetic studies have shown that the SH2 and SH3 domains of Csk enhance catalytic activity by approximately two orders of magnitude (Sondhi and Cole, Biochemistry 38:11147-11155, 1999; and Sun and Budde, Arch. Biochem. Biophys. 367:167-172, 1999). Such findings suggest that these domains play an important role in organizing the catalytic residues in the active site. Indeed, two regions near the interface between the SH2 and kinase domains display protection in the presence of AMPPNP (see FIG. 11B). Probes corresponding to helix αA in the SH2 domain (E93-L103, see FIG. 10) and a portion of the SH2-kinase linker region (Y184-N191, see FIG. 10) are protected by AMPPNP. In Csk, a short helix within the linker makes contacts with the kinase domain through helix αC. The coordinate protection in both the SH2-kinase linker and helix αC suggests that a “communication pathway” between the SH2 domain and active site is critical for catalytic function. Owing to protection by nucleotide of helix αA, this pathway may involve changes in the SH2 domain in addition to rigid domain-domain movements.

iii. Phosphorylation-Driven Motions.

The data presented thus far indicate that AMPPNP and presumably ATP induce both local and long-range movements in the kinase and neighboring SH2 domain (see FIG. 11B). It has been shown that structural changes in Csk limit the rate of release of ADP, a phenomenon that regulates function. To probe whether the ADP-bound complex populates a unique conformation compared to the ATP-bound form, deuterium incorporation into Csk in the presence of ADP was studied. These studies are directed at localizing any structural effects induced by substrate phosphorylation to specific regions in the polypeptide chain, a pursuit that could offer insights into the nature of the slow, rate-limiting structure changes in Csk. FIG. 11B displays the protected probes for Csk in the presence of ADP. While many of the probes protected by AMPPNP are equally protected by ADP, several key probes observe noticeable differences. In the kinase domain, the glycine-rich loop is more protected by ADP than AMPPNP (L200-M21 in FIG. 10). This could result partly from structural changes in the loop after phosphoryl transfer to the substrate. While it is difficult to know the molecular nature of this change, it is conceivable that the loop more adequately covers the diphosphate moiety of ADP compared to the triphosphate in ATP. Such motions may be necessary for configuring the γ phosphate into a productive form.

In addition to local effects on the glycine-rich loop, ADP has profound effects on the activation loop. In this region, ADP protects the loop to a lower extent than AMPPNP (G334-L358; FIGS. 10 and 11B), suggesting that this region of the kinase domain may exhibit higher flexibility or solvent exposure after the delivery of the y phosphate. This effect is not localized to the activation loop but rather is coupled to other motions within the large lobe. For example, helix αG is less protected in the presence of ADP than AMPPNP, a phenomenon that may reflect synchronous motions in this location (G383-G402, FIGS. 10 and 11B). Whatever the cause, it is clear that delivery of the phosphoryl donor has pervasive effects on the kinase domain. These motions are also coupled to changes in two interfacial probes. The probes corresponding to helix αA in the SH2 domain and the SH2-kinase linker are more flexible in the presence of ADP than AMPPNP (see FIG. 11B). Again, as with AMPPNP binding, ADP has long-range effects on the solution conformation of Csk. The nature of these long-range effects are markedly different depending on the presence of the γ phosphate in the nucleotide pocket. Such a striking contrast between hydrogen exchange properties as a function of substrate (AMPPNP) and product (ADP) may result from distinct conformational states. Since conformational dynamics limit substrate processing in this enzyme, the definition of critically affected regions will be useful for the understanding of protein phosphorylation.

### EXAMPLE 9

### DXMS Analysis Used to Elucidate the Effects of cAMP and Catalytic Subunit Binding on cAPK Type IIβ Solvent Accessibility

A. Background.

A myriad of physiological processes are controlled by the stimulatory effects of cAMP on cAMP-dependent protein kinase (cAPK). The regulatory (R) subunits of cAPK serve as negative regulators of cAPK, as the inactive kinase exists as a tetramer composed of an R-subunit dimer bound to two catalytic (C) subunits. Binding of two cAMP molecules to each R-subunit causes dissociation of the holoenzyme complex and releases an active C-subunit. The R-subunits are known to exist in either one of two physiological states: in complex with the C-subunit or free and cAMP-saturated. A cAMP-free and C-subunit free state is believed to only exist transiently following translation due to the high affinity for cAMP and the intracellular cAMP concentrations.

Two general classes of R-subunits, type I and type II, are known to exist and differ by autophosphorylation, molecular weight, disulfide cross linkage, and cellular localization. Each type of R-subunit can be further classified as either α or β, which differ by tissue distribution and antigenicities. Thus there are four isoforms of the R-subunits. Despite these molecular and cellular differences, all four isoforms possess a conserved and well-defined domain structure comprised of an amino-terminal dimerization/docking domain, two-tandem cAMP binding domains (designated A and B) at the carboxy-terminus, and a variable, interconnecting linker region. The linker region contains a substrate-like inhibitor sequence that docks to the active site deft of the C-subunit and each cAMP-binding domain contains a highly conserved phosphate binding cassette that binds 1 cAMP molecule. In addition to the molecular and cellular differences, the R-subunit isoforms also exhibit distinct structural differences.

Activation of cAPK is a triad of the C-subunit, cAMP, and the R-subunit. The R-subunit is at the center of this triad, as it toggles between a cAMP-bound state and a C-subunit-bound, cAMP-free state. Understanding the conformational changes induced upon binding of cAMP and the C-subunit binding to the R-subunit is essential in understanding the mechanism of cAPK activation, as a series of conformational changes is believed to be critical for holoenzyme dissociation.

Conformational changes upon cAMP binding to domain A, cA, and domain B, cB, have been observed by numerous methods, including fluorescence, circular dichroism, and cysteine sulfydryl reactivity studies. Binding of cAMP leads to a general tightening of the domain where binding occurs and also alters the conformation of the second domain. This is evidenced by the increased kₐ and decreased K_{d} for the cB domain when the cA domain is vacant Additionally, when the R-subunit is bound to C-subunit, the cB domain must be saturated before cAMP can bind to the cA domain. The specific conformational changes that occur upon nucleotide binding must be subtle, however, because neutron scattering data of an RIIα N-terminal deletion mutant did not show any large scale conformational changes upon cAMP binding.

Conformational changes in the R-subunits have also been observed upon C-subunit binding. Chemical modification studies on RIIα and RIα and limited proteolysis studies on RIIα identified residues whose reactivity was dependent on the presence or absence of the C-subunit. The C helix of the RIα cA domain was identified as a molecular switch between the cAMP-bound or the C-bound conformations, implying that this helix is essential for toggling between these two distinct conformations. Structural studies, such as neutron scattering, have also highlighted large-scale conformational changes in the R-subunits upon C-subunit binding. Attempts at solving the crystallographic structure of the RIα holoenzyme using molecular replacement of the cAMF-bound nucleotide binding domains were unsuccessful, suggesting a significantly different conformation of the cAMP-free cA domain compared to the cAMP-bound cA domain.

It is clear that conformational changes do occur upon binding of cAMP or the C-subunit but the identity of the specific residues that undergo these changes are still unknown. Amide H/²H exchange measured by mass spectrometry is one technique available for analysis of conformational changes in proteins. In this example, DXMS is used to examine the solvent accessibility of the RIIa isoform in each of the two physiological states: complexed with C-subunit (R₂C₂) or free and saturated with cAMP (R₂cAMP₄).

In contrast to the ubiquitous and well-characterized RIα isoform, the RIIβ isoform is unique because it is selectively expressed as the predominant R isoform in the brain and adipose tissue of a variety of mammals, with limited expression elsewhere. RIIβ is also believed to be adapted for the metabolic regulation and cell functions in the central nervous system. Knockout of the RIIβ gene in mice underscored the physiological importance of this isoform, as the mice displayed a lean, obesity-resistant phenotype.

Because it is necessary to separate the conformational effects of removing cAMP from the effects of C-subunit binding, three states of the RIIβ isoform have been examined in this example: cAMP-free, cAMP-bound, and C-subunit-bound (holoenzyme) RIIβ. Comparison of the results from these analyses reveals that binding of either cAMP or C-subunit results in unique changes in solvent accessibility within the protein such that a C-subunit bound RIIβ subunit is not conformationally identical to a cAMP-free RIIβ subunit.

B. Results of DXMS Analysis or RIIβ.

i. Tuning of RIIβ Proteolytic Fragmentation.

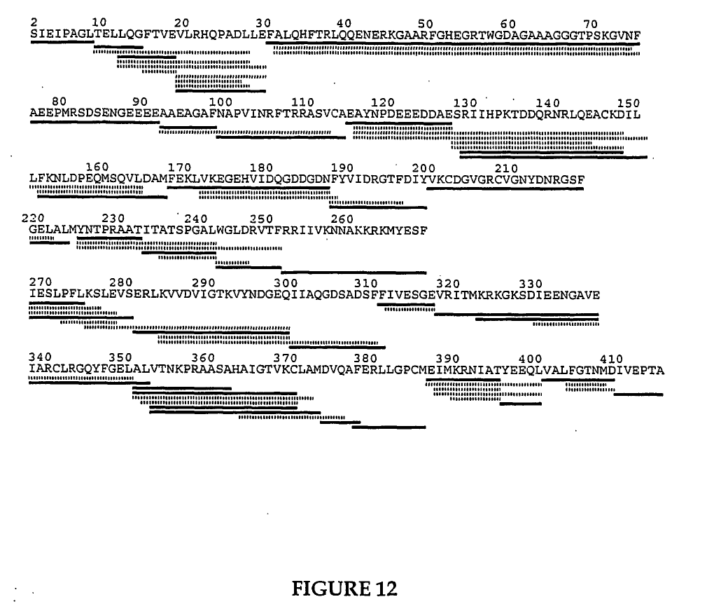

Digestion conditions that produced RIIβ fragments of optimal size and distribution for exchange analysis were established before the exchange experiments. These conditions generated 82 identified and analyzed peptides (see FIG. 12). Thirty-eight of the identified peptides, still representing >99% of the entire sequence (414 out of 415 amino acids), were used in the following analysis (bold lines in FIG. 12). Since both the amino group of the first amino acid and the amide hydrogen of the second amino acid exchange too rapidly to retain deuterons during the experiment, the total number of amide hydrogens followed by this study was 346 out of 406 non-proline residues (85%). Each peptide was numbered and discussed in terms of the amide residues whose exchange we could theoretically observe (i.e., the length of the peptide minus the first 2 amides).

ii. Deuterium Incorporation into cAMP-Bound RIIβ.

The hydrogen/deuterium exchange experiment coupled with proteolysis and mass spectrometry revealed the solvent accessibility of full length RIIβ and complemented the NMR and crystallographic studies on the dimerization/docking domain and cAMP-binding domains, respectively. A relatively slow exchanging, N-terminal region corresponded to helices I and II of the D/D domain. An approximately 100 amino add long fast exchanging region was mapped to the linker region. This region has been found to be extended, perhaps with little or no structure, solvent accessible, and very mobile, thus the fast exchange was not surprising. It is noteworthy that the deletion mutant (Δ1-111) which produced the crystals used in the crystallographic structure lacks this highly dynamic linker region.

A large slow exchanging region corresponded to the cAMP-binding domains. The slowest exchanging regions in cAMP-binding domains are residues 191-200, 222-224, 228-233, 236-242, 245-250, and 341-374, all of which are either β-sheets or α-helices in or near the PBCs. Three fast exchanging regions within the cAMP-binding domains were identified. Residues 276-281 and 390-416 correspond to a loop region and the C-terminus, respectively. Residues 326-338 correspond to a region within the cB domain (residues 326-333) whose electron density is not sufficiently well defined, suggesting that it is a dynamic region with little or no structure.

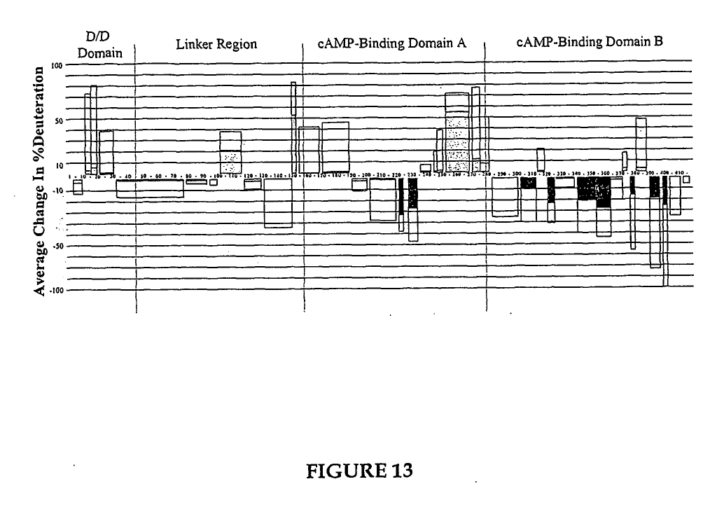

The change in solvent accessibility of the RIIβ peptides upon binding of either cAMP or C-subunit was determined (see Table 3). FIG. 13 shows a graphical representation of the solvent accessibility differences between the two physiological states, cAMP-bound and C-subunit bound. To classify the solvent accessibility of a given peptide as significantly different between these two states, the change in % deuteration must be >10% for at least 2 time points. Peptides within both cAMP-binding domains demonstrated increased deuteration upon C-subunit binding, but the majority of these peptides localized to the cB domain. Peptides that were less deuterated upon C-subunit binding were spread across the entire protein but were concentrated in the cA domain. A number of peptides exhibiting no difference in deuteron incorporation between the cAMP-bound and holoenzyme forms of RIIβ can also be identified in all subdomains.

TABLE 3
Maximum Change in % Deuteration Upon cAMP or C-Subunit Binding to RIIβ

| | | | | |
|---|---|---|---|---|
| Peptide^{a} | Residues^{b} | R₂(cAMP)₄ − R₂C₂^{cf} | R₂ − R₂(cAMP)₄^{df} | R₂ − R₂C₂^{ef} |

Dimerization/Docking Domain

| | | | | |
|---|---|---|---|---|
| 2-9 | 4-9 | −7% | 5% | −5% |
| 10-15 | 12-15 | 8% | −12% | −12% |
| 13-19 | 15-19 | 19% | −20% | −3% |
| 20-30 | 22-30 | 2% | −5% | −4% |

Linker Region

| | | | | |
|---|---|---|---|---|
| 31-76 | 33-76 | −3% | 1% | 3% |
| 77-92 | 79-92 | −2% | −4% | −5% |
| 93-99 | 95-99 | −4% | −8% | −5% |
| 100-115 | 102-115 | 38% | −3% | 36% |
| 116-128 | 118-128 | −7% | −5% | −6% |
| 129-149 | 131 -149 | −3% | 3% | 4% |
| (130-152)-(130-149) | 150-152 | 76% | −27% | 61% |

cAMP-Binding Domain A

| | | | | |
|---|---|---|---|---|
| 153-168 | 155-168 | −4% | 3% | −4% |
| 169-188 | 171-188 | 5% | 2% | 5% |
| 189-200 | 191-200 | −4% | −2% | −4% |
| 201-219 | 203-219 | −5% | 6% | 4% |
| 220-224 | 222-224 | −40% | 42% | −29% |
| 226-233 | 228-233 | −36% | 18% | −21% |
| 234-240 | 236-242 | 2% | 4% | 5% |
| 243-246 | 245-246 | 6% | 9% | 11% |
| (243-250)-(243-246) | 247-250 | 7% | −5% | 3% |
| 251-268 | 253-268 | 66% | 6% | 65% |
| 269-275 | 271-275 | 24% | −9% | 28% |
| (269-281)-(269-275) | 276-281 | 19% | 4% | 20% |

cAMP-Binding Domain B

| | | | | |
|---|---|---|---|---|
| 282-300 | 284-300 | −4% | 13% | 9% |
| 301-312 | 303-312 | −15% | −10% | −16% |
| 312-318 | 314-318 | 5% | −6% | −4% |
| 324-338 | 326-338 | −3% | −8% | −10% |
| (319-338)-(324-338) | 321-325 | −31% | 7% | −35% |
| 339-353 | 341-353 | −30% | 16% | −14% |
| 352-363 | 354-363 | −36% | 23% | −17% |
| (352-371)-(352-363) | 364-371 | −3% | 9% | 7% |
| (354-374)-(354-371) | 372-374 | 10% | −16% | 12% |
| 375-379 | 377-379 | −33% | 10% | −29% |
| 379-387 | 381-387 | 20% | 10% | 25% |
| 388-396 | 390-396 | −45% | 8% | −42% |
| 397-401 | 399-401 | −55% | 22% | −33% |
| 402-410 | 404-410 | 6% | 7% | 9% |
| 411-416 | 413-416 | −2% | −3% | −4% |

^{a}Peptide(s) analyzed. In the case of two peptides, subtraction method was used to sublocalize deuteriums incorporated.
^{b}Residues actually monitored by analyzing deuterium incorporation into the peptide.
^{c}Deuteration difference between cAMP-bound RIIβ and holoenzyme (positive if cAMP-bound RIIβ is more deuterated).
^{d}Deuteration difference between cAMP-free and cAMP-bound RIIβ (positive if cAMP-free RIIβ is more deuterated).
^{e}Deuteration difference between cAMP-free RIIβ and holoenzyme (positive if cAMP-free RIIβ is more deuterated).
^{f}Numbers in bold are considered to be a significant change.

iii. Dimerization/Docking Domain.

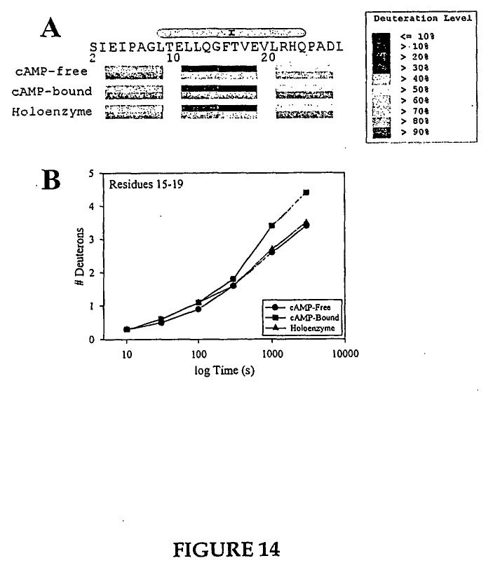

The dimerization/docking domain of RIIβ is comprised of residues 1-45. Helix I is solvent inaccessible and is flanked by a solvent accessible N-terminus and turn that leads into Helix II (see FIG. 14A). Residues 15-19 (Helix I) showed an equivalent level of deuteration for the cAMP-free and holoenzyme forms but an increase in deuteration at the longer time points (1000 s and 3000 s) for the cAMP-bound form (see FIG. 14B). This portion of Helix I, therefore, is sensitive to the addition of cAMP.

iv. Linker Region.

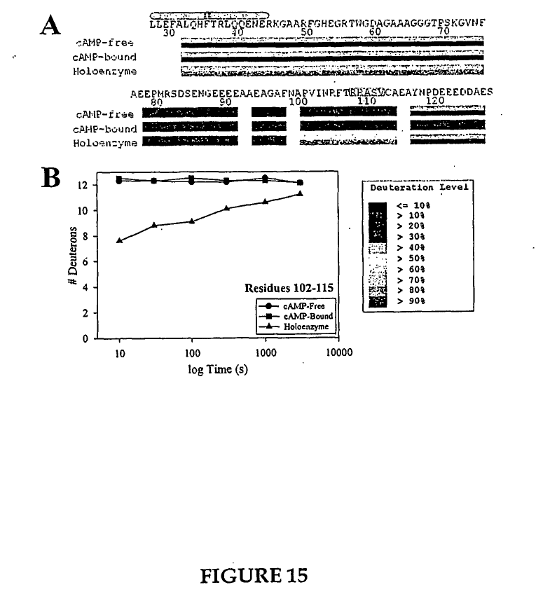

The linker region is highly solvent accessible in all three forms of RIIβ (see FIG. 15A). A localized region of protection (approximately 5 amides) upon C-subunit binding was observed for residues 102-115 (see FIG. 15B). This protection is due exclusively to C-subunit binding, as there was no observed difference in amide exchange between the cAMP-bound and cAMP-free forms. This result is consistent with the fact that this peptide includes the pseudosubstrate site.

cAMP-binding Domains.

Residues within the cAMP-binding domains whose solvent accessibility was monitored are highlighted on the RIIβ crystallographic structure. The structure includes residues 130-157 (αχₙ and αχₙ′), which are technically part of the linker region but for this report will be discussed with the cAMP-binding domains (residues 158-416).

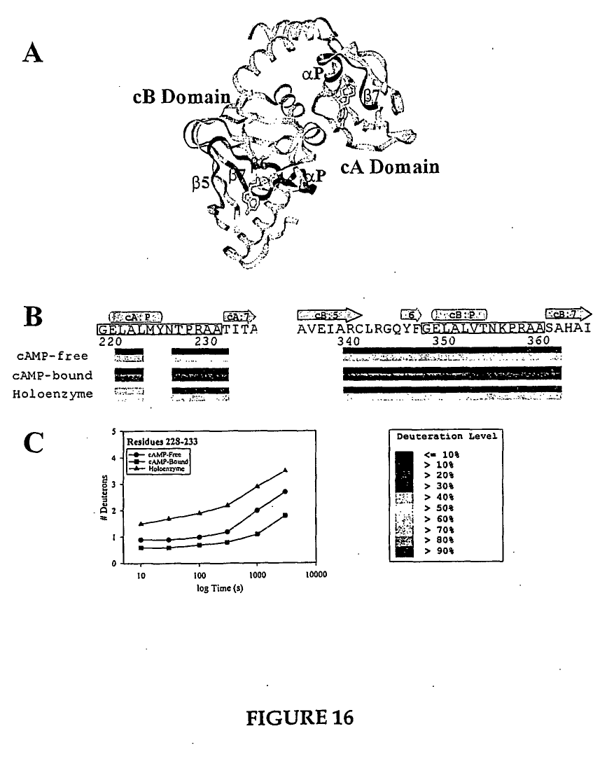

As expected, the removal of cAMP leads to increased deuteration of peptide fragments that comprise the PBCs (see FIG. 16A). These peptides, which include residues 222-224 (cA:αP), 228-233 (cA:PBC), 341-353 (cB:β6/αP), 354-363 (cB:PBC), are generally solvent inaccessible at early time-points (see FIG. 16B). At increasing time points, all of these peptides showed further deuteration upon C-subunit binding than in the cAMP-free state (see FIGS. 16B and 16C), suggesting that formation of the holoenzyme complex leads to a further opening of the PBCs.

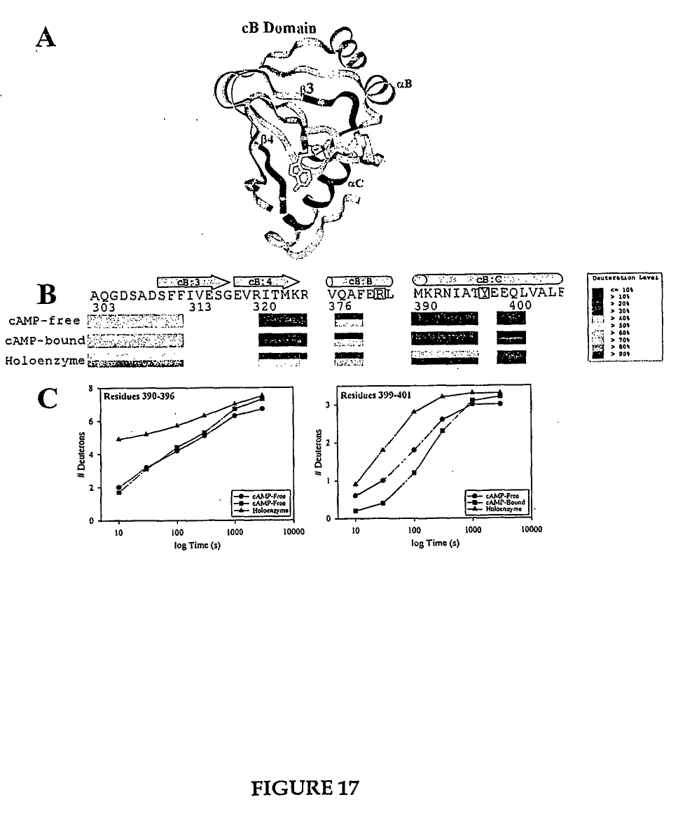

Additionally, the amide exchange of 5 peptides within the cB domain but not part of the PBC, residues 303-312 (cB:β3), 321-325 (cB:β4), 377-379 (cB:αB), 390-396 (cB:αC), and 399-401 (cB:αC), was affected by C-subunit binding (see FIG. 17A). These residues are moderately solvent accessible (10-40%) at the initial time point (see FIG. 17B) and showed increased solvent accessibility upon C-subunit binding compared to the cAMP-free state during the time course (see FIGS. 17B and 17C). With the exception of residues 399-401, the solvent accessibility of these peptides is increased upon C-subunit binding but is unaffected by just cAMP removal. Residues 399-401, alternatively, are sensitive to both cAMP and C-subunit binding, as removal of cAMP leads to an increase in amide exchange and C-subunit binding leads to further amide exchange than in the cAMP-free state.

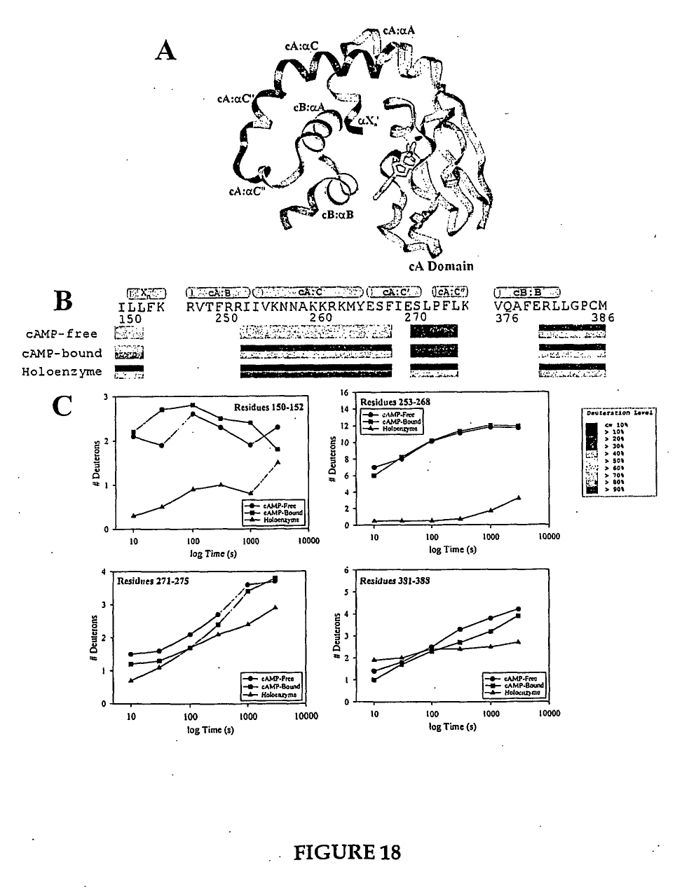

A number of peptides demonstrated a decrease in amide exchange upon C-subunit binding (see FIG. 18A). These peptides show low to moderate solvent accessibility at the initial time point (see FIG. 18B). A dramatic decrease in amide exchange is observed in residues 150-152 (αχₙ′, immediately preceding the cA domain) and 253-268 (cA:αC, cA:αC′) upon C-subunit binding (see FIG. 18C). The decrease in exchange for 253-268 appears to be due exclusively to C-subunit binding, as no difference in amide exchange upon cAMP binding was observed. Residues 150-152 showed limited protection from cAMP binding in addition to C-subunit protection. Residues 271-275 (cA:αC′), 276-281 (cB:βA), and 381-387 (cB:βB/αC) also showed decreased amide exchange that appears to be due exclusively to C-subunit binding (see FIG. 18C). The extent of protection by C-subunit, however, is not as large as for residues 150-152 and 253-268.

C. Discussion of DXMS Analysis of RIIβ.

i. Dimerization/Docking Domain is Sensitive to the Absence of cAMP.

The number of deuterons incorporated by residues 15-19 (helix I) was dependent on the presence or absence of cAMP, including cAMP removal by holoenzyme formation. Because this peptide is exclusive of sites where cAMP is known to bind, this indicates that inter-domain “cross talk” exists in RIIβ such that conformational changes upon ligand binding are transmitted from the binding site to other regions within the protein. Additionally, helix I of the dimerization/docking domain provides the docking site for A-Kinase Anchoring Proteins (AKAPs). The sensitivity of residues 15-19 to cAMP binding means that cAMP binding could affect RIIβ subcellular localization by the AKAPs.

ii. Sites of Protection by C-Subunit Binding are Observed in the Pseudosubstrate Site.

Residues 102-115, which contain the pseudosubstrate site (residues 109-113), show protection upon C-subunit binding that is believed to result from direct interactions with the C-subunit. At the shortest time point (10 s), the difference in number of deuterons incorporated between cAMP-bound RIIβ and holoenzyme was 5 deuterons, indicating a minimum of 5 amide hydrogens were protected upon C-subunit binding. As the pseudosubstrate site is 5 amides long, this 5 amide protection can be attributed to result from C-subunit binding to the pseudosubtrate site. Previous RIα studies have suggested that the P4 to P-11 residues amino-terminal to the pseudosubtrate site also interact with C-subunit. The results presented herein do not indicate any C-subunit protection beyond the pseudosubstrate site. The high levels of deuteration for the linker region (residues 31-128) at the earliest time point indicate that most of the exchange events occurred before the time window we employed. Additional protection may have been observed if we had utilized shorter time points.

iii. Sites of Protection by C-Subunit Binding are Observed in Both cAMP-Binding Domains.

Residues 253-268, which encompass the C-helix of the cA domain, show a dramatic decrease in solvent accessibility upon C-subunit binding that is believed to result from direct interactions with the C-subunit (see FIG. 18C). Previous studies on RIα indicated that the cA domain C-helix provides a peripheral docking site for C-subunit binding to the R-subunits. In addition to being a determinant for high affinity binding to the C-subunit, the C-helix of RIα is protected from solvent exchange upon C-subunit binding. In RIIβ, residues 253-268 of the C-helix showed a dramatic protection upon C-subunit binding such that, compared to the cAMP-bound protein, at least 10 amide hydrogens are protected from deuteration upon C-subunit binding. The absence of protection by cAMP binding in this region suggests that this difference is due exclusively to C-subunit binding. A peripheral C-subunit docking site thus appears to also exists in the RIIβ cA domain C-helix.

Residues 150-152 (αχₙ′), which sit alongside the cA domain C-helix, also showed significantly decreased solvent accessibility in the C-subunit bound state compared to the cAMP bound state of RIIβ. The strong protection observed for these residues suggests that αχₙ′ may also provide a site of direct interaction with the C-subunit. Interestingly, residues 155-165 (cA:αA) showed no observable difference in amide exchange upon C-subunit binding (see FIG. 13). Single-site mutational analysis of this region in RIα suggested that E140 and D143 are required for high-affinity binding of RIα to the C-subunit The absence of protection in the RIIβ A-helix suggests an isoform-specific C-subunit interaction surface in RIα and RIIβ.

The decreased solvent accessibility upon C-subunit binding observed in residues 271-275 and 276-281, which comprise the cA domain C″ helix and the turn following this helix, respectively, is believed to be a propagated effect from interactions at the peripheral C-subunit binding site of the cA domain C-helix. The decrease in exchange is not attributed to direct binding primarily because the decrease in solvent accessibility is not as dramatic as that observed for residues 253-268 and 150-152. Binding of the C-subunit, then, affects the entire cA domain C-helix and not just peripheral binding site within the helix.

The amide exchange behavior of residues 381-387 (cA:B domain B and C helices) is complicated (see FIG. 18). Compared to the cAMP-bound and cAMP-free states, upon C-subunit binding the peptide was more deuterated at the 10 s and 30 s time points while it was less deuterated at the longer time points (300-3000 s). This indicates that there are two types of amide hydrogens in this peptide: one with exchange rates that are slowed and another with exchange rates that are accelerated by C-subunit binding. The influence of C-subunit binding on residues 381-387, of which Arg381 and Leu382 are part of the cB domain B-helix, is striking because of the interaction between the cB domain B-helix and the cA domain. Arg381 stacks against the adenine ring of the cAMP molecule sequestered in the cA domain binding pocket, thus protection of residues 381-387 suggests an intimate connection between C-subunit binding and the cA domain binding pocket. Thus, the C-subunit binding to the peripheral binding site of the cA domain C-helix could induce a conformational change in the cB domain B-helix. This would in turn alter the stacking of Arg381 against the cAMP molecule within the cA domain binding pocket and facilitate the release of cAMP. Furthermore, the cB domain C-helix stacks against the cAMP molecule sequestered in the cB domain binding pocket and acts as a lid for the β-barrel to shield the cAMP molecule from solvent. A conformational change within residues 381-387, which includes the turn leading into the cB domain C-helix, could also facilitate the release of cAMP bound to the cB domain

iv. C-Subunit Binding Propagates Changes in Solvent Accessibility Within the cAMP-Binding Domain β-Barrel Subdomain.

As expected, the PBCs showed increased solvent accessibility upon cAMP removal. Each cassette (residues 220-232 and 349-361) has three backbone amides and two amino acid sidechains within hydrogen bonding distance of the cAMP molecule. Binding of the C-subunit results in an increased level of amide exchange within the cAMP-binding pockets that is beyond the exchange resulting from simple removal of cAMP. Therefore, C-subunit binding propagates additional conformational changes to the PBCs that may facilitate the release of cAMP. The same trend in amide exchange is also observed for residues 399-401 of the cB domain C-helix. The fact that residues within this cB domain “lid” become more solvent accessible upon holoenzyme formation further supports the hypothesis that C-subunit binding may facilitate the release of cAMP and “prime” the cB domain for future cAMP binding.

Interestingly, residues 303-312, which exhibited an increase in amide exchange, include 2 conserved Asp residues (Asp306 and Asp309) that are indirectly linked to the cB domain cAMP molecule through a conserved Arg residue (Arg359). This Arg is essential for cAMP binding as it interacts with the cAMP exocyclic oxygen. The speculation that C-subunit binding could influence cAMP binding through a Asp306/Asp309-Arg359-cAMP network again suggests that C-subunit binding could facilitate the release of cAMP from the cB domain.

Additional changes in the amide exchange of peptides exclusive of the PBCs were also observed within the cAMP-binding domains, but they were observed only in the cB domain. The cB domain appears to be more malleable upon C-subunit binding than the cA domain. The influence of the C-subunit on domain B is somewhat surprising because the cB domain was believed to act solely as a gatekeeper to control cAMP binding to the cA domain. Furthermore, deletion of the entire cB domain in RIα had little effect on binding of RIα to the C-subunit. The effect of C-subunit binding on amide exchange within the RIIβ cB domain suggests that domain B may play a role in interactions with the C-subunit. The increased solvent accessibility within the cB domain upon C-subunit binding may help to “prime” the domain for the inevitable dissociation of the holoenzyme.

While the invention has been described and exemplified in sufficient detail for those skilled in this art to make and use it, various alternatives, modifications, and improvements should be apparent without departing from the spirit and scope of the invention. The present invention is well adapted to carry out the objects and obtain the ends and advantages mentioned, as well as those inherent therein

The examples provided here are representative of preferred embodiments, are exemplary, and are not intended as limitations on the scope of the invention Modifications therein and other uses will occur to those skilled in the art. These modifications are encompassed within the spirit of the invention.

The disclosure of all publications cited above are expressly incorporated herein by reference, each in its entirety, to the same extent as if each were incorporated by reference individually.

1. A method of determining, at a resolution of about 1-5 amino acid residues, the position of a peptide amide hydrogen that has been labeled with an isotope of hydrogen other than ¹H within a protein of interest, said method comprising:
determining the quantity of isotope and/or rate of exchange of hydrogen at a peptide amide hydrogen with said isotope by generating a population of sequence-overlapping endopeptidase fragments of said labeled protein under conditions of slowed hydrogen exchange and then deconvoluting said fragmentation data acquired from said endopeptidase fragments.

2. A method according to claim 1, wherein said endopeptidase fragments are generated by cleaving said protein with an endopeptidase selected from the group consisting of a serine endopeptidase, a cysteine endopeptidase, an aspartic endopeptidase, a metalloendopeptidase, and a threonine endopeptidase.

3. A method according to claim 1, wherein said endopeptidase fragments are generated by cleaving said protein with pepsin.

4. A method according to claim 1, wherein said endopeptidase fragments are generated by more than one endopeptidase used in combination.

5. A method according to claim 1, wherein said endopeptidase fragments are generated by cleaving said protein with newlase or Aspergillus protease XIII.

6. A method according to claim 1, wherein said endopeptidase fragments are generated by cleaving said protein with an acid-tolerant Aspergillus protease.

7. A method according to claim 1, wherein said isotope is deuterium.

8. A method according to claim 7, wherein the presence or absence and/or quantity of said isotope on an endopeptidase fragment is determined by measuring the mass of said endopeptidase fragments.

9. A method according to claim 8, wherein said measuring is performed using mass spectrometry.

10. A method according to claim 1, wherein said endopeptidase fragments are generated at a pH of about 1.8-3.4.

11. A method according to claim 1, wherein said endopeptidase fragments are generated at a pH of about 2-3.

12. A method according to claim 1, wherein said endopeptidase fragments are generated at a pH of about 2.0-2.5.

13. A method according to claim 1, wherein said endopeptidase fragments are generated at a pH of about 2.5-3.0.

14. A method according to claim 1, wherein said endopeptidase fragments are generated in less than five minutes.

15. A method according to claim 1, wherein said endopeptidase fragments are generated in about one minute or less.

16. A method according to claim 1, wherein said endopeptidase fragments are generated in about 40 seconds or less.

17. A method according to claim 1, further comprising the use of conditions that effect protein denaturation under slowed exchange conditions prior to generating said endopeptidase fragments.

18. A method according to claim 17, wherein said conditions comprise contacting said labeled protein with guanidine hydrochloride at a concentration of about 0.05-4 M.

19. A method according to claim 17, wherein said conditions comprise contacting said labeled protein first with guanidine thiocyanate at a concentration of about 1.5-4 M, followed by dilution into guanidine hydrochloride at a concentration of about 0.05-4 M.

20. A method according to claim 1, further comprising disrupting disulfide bonds in the labeled protein prior to generating said endopeptidase fragments.

21. A method according to claim 20, wherein said disrupting comprises contacting said labeled protein with a phosphine.

22. A method according to claim 1, wherein deconvoluting comprises:
comparing the quantity of isotope and/or rate of exchange of hydrogen at a peptide amide hydrogen with said isotope on a plurality of said endopeptidase fragments with the quantity of isotope and/or rate of exchange of hydrogen at a peptide amide hydrogen said isotope on at least one other endopeptidase fragment in said population,
wherein said quantities are corrected for back-exchange losses subsequent to the initiation of slowed exchange conditions in an amino acid sequence-specific manner.

23. A method according to claim 22, wherein labeled peptide amides are localized in an amino acid sequence-specific manner by measuring rates of exchange as a function of time under slowed exchange conditions.

24. A method according to claim 1, wherein said population of endopeptidase fragments contains a plurality of sequence-overlapping fragments, wherein more than half of the members of said population have sequences that overlap other members of said population over all but 1-5 amino acid residues.

25. A method according to claim 1, wherein a majority of members of said population of endopeptidase fragments is present in an analytically sufficient quantity to permit its further characterization.

26. A method according to claim 1, wherein determining the quantity and rate of exchange of peptide amide hydrogen(s) is carried out contemporaneously with generating a population of endopeptidase fragments.

27. A method according to claim 1, further comprising determining off-exchange rates of labeled peptide amides under conditions of slowed hydrogen exchange and random-coil conditions from a plurality of fragments and fragment differences.

28. A method of characterizing the binding site of a binding protein and/or determining peptide amides that are near residues important in the interaction between a binding protein and a high affinity binding partner therefor, said method comprising:
labeling said protein with an isotope of hydrogen other than ¹H in the presence and absence of a binding partner for said protein, and comparing the pattern of labeling obtained on said protein in the presence and absence of said binding partner,
wherein the location of label is determined by generating a population of endopeptidase fragments of said labeled protein under conditions of slowed hydrogen exchange and then deconvoluting fragmentation data acquired from said endopeptidase fragments.

29. A method according to claim 28, wherein said binding partner is a polypeptide.

30. A method according to claim 28, wherein said binding partner is a nucleic acid.

31. A method according to claim 28, wherein said binding partner is a small molecule.

32. A method according to claim 31, wherein said small molecule is selected from the group consisting of a ligand, a substrate, an inhibitor, an activator, a co-factor, and a drug.

33. A method of characterizing the binding site of a binding protein and/or of determining peptide amides that are near residues important in the interaction between a binding protein and a high affinity binding partner therefor said method comprising:
labeling said protein with an isotope of hydrogen other than ¹H;
contacting said labeled protein with a binding partner for said protein; and
off-exchanging the resulting binding pair;
wherein the location of label is determined by generating a population of endopeptidase fragments of said labeled protein under conditions of slowed hydrogen exchange and then deconvoluting fragmentation data acquired from said endopeptidase fragments.

34-35. (canceled)

36. A method of screening test compounds to determine whether any compounds mimic the interaction of a binding protein and a high affinity binding partner therefor, said method comprising:
determining which peptide amides of said protein are near residues important in said interaction according to the method of claim 28; and
determining the location of isotope(s) on the labeled peptides produced by on-exchanging said protein with said isotope of hydrogen and off-exchanging the resulting labeled protein in the presence and absence of said test compound;
wherein a similar location of isotope(s) on the labeled peptides identified in the presence of said test compound, as compared to the location of isotope(s) on the labeled peptides identified in the presence of said high affinity binding partner, is indicative of a test compound that mimics the interaction of a binding protein and a high affinity binding partner therefor.

37. (canceled)

38. A method of determining the surface conformation of a polypeptide, said method comprising:
labeling said protein with an isotope of hydrogen other than ¹H,
determining the quantity of isotope and rate of exchange of hydrogen at a peptide amide hydrogen with said isotope by generating a population of sequence-overlapping endopeptidase fragments of said labeled protein under conditions of slowed hydrogen exchange and then deconvoluting said fragmentation data acquired from said endopeptidase fragments, and
comparing the rates of exchange of a plurality of peptide amide hydrogens to determine the surface accessibility of peptide amide hydrogens.

39. A method of determining a conformational change in a polypeptide, said method comprising:
independently labeling more than one conformer of said protein with an isotope of hydrogen other than ¹H,
determining the quantity of isotope and/or rate of exchange of hydrogen at a peptide amide hydrogen with said isotope in said conformers by generating a population of sequence-overlapping endopeptidase fragments of said labeled conformers under conditions of slowed hydrogen exchange and then deconvoluting fragmentation data acquired from said endopeptidase fragments, and
comparing the fragmentation data acquired from said conformers wherein a change in said data reflective of a difference in labeling of said peptide amide hydrogen(s) is indicative of a conformational change.

40. A method of determining a conformational change in a polypeptide as a result of a change in conditions within which the polypeptide is present, said method comprising:
labeling said protein with an isotope of hydrogen other than ¹H at two or more conditions,
determining the quantity of isotope and/or rate of exchange of hydrogen at a peptide amide hydrogen with said isotope by generating a population of endopeptidase fragments of said labeled protein under conditions of slowed hydrogen exchange and then deconvoluting fragmentation data acquired from said endopeptidase fragments, and
comparing the fragmentation data acquired at each condition, wherein a change in said data reflective of a difference in labeling of said peptide amide hydrogen(s) is indicative of a conformational change in said polypeptide as a result of a change in conditions.

41. A method according to claim 40, wherein said change in conditions represents the introduction of additional molecule(s) that combine with said polypeptide.

42. A method of determining a conformational change in a polypeptide as a result of a change in conditions within which the polypeptide is present, said method comprising:
labeling said protein with an isotope of hydrogen other than ¹H;
off-exchanging said labeled protein following a change in conditions;
determining the quantity of isotope and/or rate of exchange of hydrogen at a peptide amide hydrogen with said isotope by generating a population of endopeptidase fragments of said labeled protein under conditions of slowed hydrogen exchange and then deconvoluting fragmentation data acquired from said endopeptidase fragments; and
comparing the fragmentation data acquired, wherein a change in said data reflective of a difference in labeling of said peptide amide hydrogen(s) is indicative of a conformational change in said polypeptide as a result of a change in conditions.

43. A method of determining, at a resolution of about 1-5 amino acid residues, the position of peptide amine group(s) that have been hydrogen-exchanged labeled with deuterium in a protein of known amino acid sequence, said method comprising:
(a) placing the labeled protein under conditions of slowed hydrogen exchange;
(b) fragmenting the labeled protein with endopeptidase(s) to produce a population of sequence-overlapping fragments, wherein more than half of said fragments differ by 1-5 amino acid residues;
(c) quantifying the amount of deuterium label on a plurality of members of said population by mass spectrometry; and
(d) comparing the amount of deuterium label on at least one member of said population with the amount of deuterium label on at least one other member of said population;
thereby localizing the position of the deuterium-labeled peptide amide group in the protein to within about 1-5 amino acid residues.
